(19) United States
(12) Patent Application Publication
(10) Pub. No.: US 2007/0082858 A1
(43) Pub. Date: Apr. 12, 2007

(54) Methods and compositions for the prevention of toxic side effects of aminoglycoside medications

(75) Inventors: Peter S. Steyger, Portland, OR (US); Sigrid Myrdal, Portland, OR (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Oregon Health & Science University, Portland, OR (US)

(21) Appl. No.: 11/473,300

(22) Filed: Jun. 21, 2006

(30) Foreign Application Priority Data
Dec. 21, 2004 (WO) PCT/US04/43124

(51) Int. Cl. A61K 31/7034; A61K 31/455

(52) U.S. Cl. 514/35; 514/355

(57) ABSTRACT

Compositions and methods for reducing and/or preventing antibiotic-related damage to cells of the inner ear and the kidney are provided. Such compositions and methods reduce or prevent hearing loss and kidney damage resulting from use of antibiotics, such as aminoglycoside antibiotics. The composition also find use in reducing or preventing inner ear and kidney damage associated with anti-neoplastic agents, other therapeutic drugs, and environmental factors.

### FIELD OF THE INVENTION

The present invention is directed to methods for the prevention of toxic side effects of aminoglycoside antibiotic medications and medications having molecular structures similar to aminoglycosides, more particularly to therapy targeting cation channels, especially TRP-like cation channels including TRPV1, to prevent the entry of antibiotic medications into cells of the human body, especially the cells of the kidney and of the inner ear.

### BACKGROUND OF THE INVENTION

Ototoxicity and nephrotoxicity are side effects of certain aminoglycoside antibiotic medications, such as gentamicin. The toxic side effects of aminoglycosides are well-known, but the mechanism of that cytotoxicity is poorly characterized.

Aminoglycoside antibiotics are vital for the treatment of serious bacterial infections. However, in some patients, the antibiotics have severe toxic effects, particularly on kidney function and on the auditory system. The toxic effects of these drugs are often the limiting factor for their therapeutic usefulness. For example, antibacterial aminoglycosides such as gentamicins, streptomycins, kanamycins, tobramycins, and the like are known to have serious toxicity, particularly ototoxicity and nephrotoxicity, which reduce the usefulness of such antimicrobial agents (see Goodman and Gilman's The Pharmacological Basis of Therapeutics, 6^{th} ed., A. Goodman Gilman et al., eds; Macmillan Publishing Co., Inc., New York, pp. 1169-71 (1980) or most recent edition). Aminoglycoside antibiotics are generally utilized as broad spectrum antimicrobials effective against, for example, gram-positive, gram-negative and acid-fast bacteria. Susceptible microorganisms include Escherichia spp., Hemophilus spp., Listeria spp., Pseudomonas spp., Nocardia spp., Yersinia spp., Klebsiella spp., Enterobacter spp., SalMycobacteria spp., Shigella spp., and Serratia spp. Nonetheless, the aminoglycosides are used primarily to treat infections caused by gram-negative bacteria, such as meningitis and, for instance, in combination with penicillin for the synergistic effects. As implied by the generic name for the family, all the aminoglycoside antibiotics contain aminosugars in glycosidic linkage.

Otitis media is a term used to describe infections of the middle ear, which infections are very common, particularly in children. Typically antibiotics are systemically administered for infections of the middle ear, e.g., in a responsive or prophylactic manner. Systemic administration of antibiotics to combat middle ear infection generally results in a prolonged lag time to achieve therapeutic levels in the middle ear, and requires high initial doses in order to achieve such levels. These drawbacks complicate the ability to obtain therapeutic levels and may preclude the use of some antibiotics altogether. Systemic administration is most often effective when the infection has reached advanced stages, but at this point permanent damage may already have been done to the middle and inner ear structure. Clearly, ototoxicity is a dose-limiting side-effect of antibiotic administration. For example, nearly 75% of patients given 2 grams of streptomycin daily for 60 to 120 days displayed some vestibular impairment, whereas at 1 gram per day, the incidence decreased to 25% (U.S. Pat. No. 5,059,591). Auditory impairment was observed: from 4 to 15% of patients receiving 1 gram per day for greater than 1 week develop measurable hearing loss, which slowly becomes worse and can lead to complete permanent deafness if treatment continues.

The loss of sensory hair cells in the cochlea has been attributed to aminoglycoside ototoxicity. Apoptosis of sensory hair cells of guinea pigs was observed following chronic treatment with aminoglycoside (Nakagawa et al., Eur. Arch. Otor., 254:9-14, 1997; Nakagawa et al., Acta Otol., 255(3):127-131, 1998). Studies have assessed the protective effect of various polypeptides on sensory hair cells in the cochlea. (See, for example, Stacker, et al., 1997, Int. J. Dev. Neuroscience 15:553-562; Low et al., J. Cell. Physiol. 167:443-450, 1996; and Ernfors et al., Nature Medicine, 2:463-467, 1996). Ernfors et al. noted that, although the peptide NT-3 is a potent factor for preventing the degeneration of spiral ganglion neurons, NT-3 “insufficiently protects the hair cells” (Ernfors et al., Nature Medicine, 2:463-467, 1996).

Platinum-based cytotoxic agents include, but are not limited to, cisplatin and carboplatin. Cisplatin is a widely used antitumor drug which causes structural changes in the inner ear and peripheral sensory neuropathy. Hearing loss due to cisplatin is usually permanent and cumulative. Nephrotoxicity, also induced by aminoglycoside antibiotics and by drugs such as cisplatin, has important consequences for the patient, with potential permanent loss of 50% or more of normal renal function (Kemp, et al. J. Clin. Oncology, 14:2101-2112, 1996). This can produce serious disability, requiring the need for dialysis in severe cases, and early mortality. It also has important consequences for the ability of the patient to be safely treated with medications such as antibiotics that are themselves renally toxic or require adequate renal function for elimination from the body.

Although certain off-label uses of some medications have been shown to be effective in combating the toxic effects of antibiotics, these medications are not always effective for all patients, and there is a substantial need in the art for a way to provide these much-needed aminoglycoside medications without putting the auditory and renal functions of the patient in distress. In particular, the most commonly used mechanisms for preventing toxic side effects from aminoglycoside medications is through use of a pharmaceutical composition composed of agents chosen for their potential to prevent cell death once the drug has entered the cells of the inner ear and of the kidney. Such treatments are generally ineffective, because the cells of the kidney and of the inner ear are so sensitive to the toxic effects of these drugs that once these cells have started on the pathway toward cell death, significant damage has already been done to organ function before the pathway can be halted.

Cell death inhibitors include anti-oxidants, salicylate (Sha and Schacht, 1999); inhibitors of caspase-3 (Liu et al., 1998); inhibitors of c-Jun kinase (Ylikosi et al., 2002), and inhibitors of calpain (Ding et al., 2002). These agents act after ototoxic drug uptake and subsequent toxicity within the cell.

There exists a need in the art for means to prevent and/or reduce the incidence and/or severity of inner ear and kidney damage due to chemical agents. Of particular interest are those conditions arising as an unwanted side-effect of ototoxic and nephrotoxic therapeutic drugs including cisplatin and its analogs, aminoglycoside antibiotics, salicylate and its analogs, and loop diuretics. In addition, there exists a need for methods that will allow higher and thus more effective dosing with these oto- and nephrotoxicity inducing pharmaceutical drugs, while concomitantly preventing or reducing toxic effects caused by these drugs. There is a need in the art for a method that provides a safe, effective, and prolonged means for prophylactic or curative treatment of hearing impairments related to inner ear or kidney tissue damage, loss or degeneration, particularly ototoxin or nephrotoxin induced, and particularly involving the cells of the inner ear and of the kidney.

### SUMMARY OF THE INVENTION

Methods for preventing the entry of aminoglycoside drugs into mammalian cells are provided. These methods include blocking uptake by using cation channel regulating drugs to reduce or prevent entry of drugs through cation channels.

The invention also provides methods for identifying the mechanism of cellular uptake by which aminoglycoside antibiotics exert their oto- and nephrotoxic effects in order to develop an effective pharmaceutical cocktail to prevent uptake and thus prevent oto- or nephrotoxicity.

The invention further provides for the identification and testing of compounds capable of inhibiting or interfering with uptake of aminoglycoside drugs, by utilizing the binding characteristics of the TRPV1 channel and the structure of the specific drug, and selecting a compound the physical and chemical characteristics of which are predicted to prevent or interfere with drug entry through the channel.

In one embodiment the invention provides a method for treating a vertebrate, including mammals and humans, prophylactically to prevent or reduce the occurrence or severity of a hearing loss or balance impairment.

Another embodiment of the invention provides a method for treating a vertebrate, including mammals and humans, prophylactically to prevent or reduce the occurrence or severity of an impairment of kidney function.

It is an object of the invention to provide a method for treating a vertebrate, including mammals and humans, to prevent, reduce or treat a hearing or vestibular impairment, disorder or imbalance, particularly an impairment caused by an ototoxic drug, by administering to a mammal in need of such treatment a composition of the invention. More preferably, the hearing or vestibular impairment, disorder or imbalance is caused by a therapeutically effective amount of an antibiotic drug. Most preferably, the hearing impairment, disorder or imbalance is caused by a therapeutically effective amount of an aminoglycoside antibiotic drug.

It is another object of the invention to provide a method for treating a vertebrate, including mammals and humans, to prevent, reduce or treat an impairment, disorder or imbalance in the kidney, particularly an impairment caused by an ototoxic drug, by administering to a mammal in need of such treatment a composition of the invention. More preferably, the impairment, disorder or imbalance of the kidney is caused by a therapeutically effective amount of an antibiotic drug. Most preferably, the hearing impairment, disorder or imbalance is caused by a therapeutically effective amount of an aminoglycoside antibiotic drug.

### BRIEF DESCRIPTION OF THE DRAWINGS

FIG. 1: Consequences of loss of outer hair cells (OHCs). Frequency response curves in the afferent fibers innervating inner hair cells are broadened and elevated, indicating loss of response at the higher frequencies of sound.

FIG. 2: Opening of mechano-electrically gated transduction channels leads to a positively charged transduction current influx carried mostly by potassium ions. This influx in turn opens calcium channels in the basolateral membrane of the hair cell, depolarizing the cell and causing the release of neurotransmitters at the base of the hair cell.

FIG. 3: Modulation of GTTR uptake into cytoplasmic/nuclear compartments by calcium and pH. In all images, MDCK cells were washed twice with 0.9% NaCl, then treated as indicated for 30 sec with 1 μg/ml GTTR at 20° C., washed twice with saline, fixed with PFMT and again washed. All images were obtained using the same imaging parameters. Scale bar=20 μm. A1-A7) Cells were treated with varying concentrations of calcium in 0.9% saline show increased binding from 0 to 0.16 mM calcium with serially declining binding at higher concentrations; B1-B7) Cells were treated in PBS at varying pH, as indicated show increased GTTR binding at pH 5 and very slight increase at pH 6 with a great reduction at pH 10; C1-C7 and D1-D7) Cells treated in 0.9% saline alone or with TRPV-1 agonist or antagonists, as indicated.

FIG. 4: Modulation of GTTR uptake into cytoplasmic/nuclear compartments by TRPV1 agonists/antagonists. In all images, MDCK cells were washed twice with 0.9% NaCl, then treated as indicated for 30 sec with 1 μg/ml GTTR at 20° C., washed twice with saline, fixed with PFMT and again washed. All images were obtained using the same imaging parameters. Scale bar=20 μm. A1) GTTR alone; B1-3) Cells treated in the presence of agonist RTX show stimulation of GTTR uptake at 10⁻⁸M, and declining stimulation at higher RTX concentrations; C1-3) Cells treated in the presence of agonist anandamide also show stimulation of GTTR uptake at 10⁻⁶M and 10⁻⁵M with little or no stimulation at 10⁻⁴ M; D1-3) Cells treated in the presence of antagonist SB366791 show stimulation of GTTR uptake increasing at concentrations from 10−7 to 10⁻⁵M, A2, B4, C4, D4, E4) Cells treated with GTTR in the presence of 100 mM Ruthenium Red alone, or with agonists or antagonists at their most effective tested doses, A2) RR alone shows decreased GTTR uptake compared to A2; B4-E4) RR blocked enhanced GTTR uptake induced by all agonists and antagonists tested, with only a partial effect on anandamide stimulation.

FIG. 5: Extracellular PIP₂ prevents GTTR uptake. (A) Control cells were treated with 1 ug/ml GTTR for 1 minute. (B) When 75 μg/ml of PIP₂ was added to the extracellular media, prior to GTTR, a large reduction in GTTR uptake was observed compared to the control. This was not due to the trivial effect of fluorescence quenching, because treated cells were delipidated, as usual, by 0.5% Triton X-100 present in the fixative that should have removed the added PIP₂. Therefore, PIP₂ blocked GTTR uptake in the cells, and/or its binding at intracellular sites.

FIG. 6: Cytoplasmic and vesicular gentamicin-labeled Texas Red (GTTR) in MDCK cells treated with 1 μg/ml GTTR at 37° C. for 2.5 hours, washed and treated as described, then imaged with the aid of confocal microscopy. Control cells were treated identically with equivalent amounts of unconjugated, hydrolyzed Texas Red. All images were obtained using the same imaging parameters. A1) Live cells with numerous GTTR-loaded vesicles; A2) Cells in 4% formaldehyde containing 0.5% Triton X-100 (FATX) show no vesicular GTTR, and weak fluorescent cytoplasmic and intra-nuclear labeling; A3) Cells treated as A2, and then washed with buffer show bright cytoplasmic and intra-nuclear labeling; A4) Cells treated as A3, then treated with PIP₂ for 1.5 hours show quenched fluorescence; A5) Cells treated with PIP₂ as in A4, then delipidated with 0.5 Triton X-100 and washed show recovery of fluorescence brightness. B1) Cells in 4% formaldehyde alone show vesicular and surface labeling; B2) Cells fixed as in B 1, then washed with buffer show vesicular labeling but little surface labeling; B3) Cells fixed and washed as in B2, delipidated with 0.5% Triton X-100 and washed in buffer show bright cytoplasmic and intra-nuclear labeling, as in A3; B4) Enlarged detail from B3 showing fluorescent structure traversing the nucleus.

FIG. 7: PIP₂ quenches GTTR but not TR fluorescence. A) 3-dimensional excitation and emission scan of Texas Red. B) 3-dimensional excitation and emission scan of Texas Red in the presence of PIP₂. Note the similarity of the spectra to that seen in A). C) 3-dimensional excitation and emission scan for GTTR. D) 3-dimensional excitation and emission scan of GTTR in the presence of PIP₂. Note the reduced fluorescence emission intensities compared to C). E) Emission of GTTR at 618 mm (+/−5 mm) in the absence (red) or presence (blue) of PIP₂ at different excitation wavelengths (range 290-610 nm). F) Fluorescence intensity of GTTR at different emission wavelengths (+/−5 nm) in the absence (red) or presence (blue) of PIP₂ at a fixed excitation wavelength (587+/−5 nm).

FIG. 8: Saturability of cytoplasmic, but not vesicular GTTR fluorescence. OK or MDCK cells were treated with 1 μg/ml GTTR at 37° C. or held over ice in the presence of increasing concentrations of unlabeled gentamicin. Cells were imaged live or fixed with FATX and washed before imaging. A1-A5) At 37° C., OK cells treated with GTTR for 120 minutes show serially reduced labeling with increasing unlabeled GT concentrations. Inset in A1) shows only vesicular GTTR labeling in live OK cells treated with GTTR alone. Inset in A5) also shows no decrease in vesicular GTTR labeling in live OK cells treated with GTTR plus 4 mg/ml unlabeled gentamicin. B1-B5) OK cells held on ice during 120 minutes of GTTR treatment show reduced GTTR binding in the cytoplasm and intra-nuclear compartments (compared to 37° C., A1-A5) and reduction of binding with increasing concentrations of unlabeled GT concentrations. Inset in B1) shows no vesicular GTTR uptake in OK cells held on ice when imaged live. C1-C5) MDCK cells held on ice during 120 minutes GTTR treatment also show decreasing cytoplasmic GTTR fluorescence as concentration of unlabeled gentamicin increases.

FIG. 9: Time and temperature. OK cells treated with 1 μg/ml GTTR at 37° C. or on ice and fixed after time intervals as indicated. A1-A6). Cells at 37° C. show increased GTTR binding over time; B1-B6). Cells held over ice during treatment show increased GTTR binding over time, but less than that seen at 37° C.

FIG. 10. Chemical structure of aminoglycoside antibiotics, gentamicin and cyclosporine, and structure of cisplatin and carboplatin.

FIG. 11. GTTR fluorescence is reduced by excess free gentamicin and is not replicated by free Texas Red. (A) Typical distribution of GTTR fluorescence in saccular explants following in vitro incubation with 300 μg/ml GT/GTTR for 30 minutes. There are intensely-labeled cells at the periphery (P) of the sensory epithelium, less intensely labeled central cells [C] and negligible labeling in the extra-sensory epithelium (ES). (B) Explants incubated with 300 μg/ml GT/GTTR plus 12 mg/ml free GT display reduced GTTR fluorescence in the sensory epithelium, particularly in the peripheral regions. (C) Explants incubated with 300 μg/ml free GT plus 1.8 μM unconjugated TR (equivalent to the concentration of TR in 300 μg/ml GT/GTTR) display negligible fluorescence within the sensory epithelium. (D) Explants incubated with free TR alone also display negligible fluorescence. Scale bar in μm.

FIG. 12. GTTR is preferentially taken up by hair cells at the periphery of the saccular macula 30 minutes after addition of 300 μm/ml GT/GTTR. (A) At low magnification, FITC-phalloidin labeling reveals a distinct pattern of bright dots (arrows) that represents the sensory hair bundles perpendicular to the surface of the sensory epithelium. (B) GTTR fluorescence occurs throughout the sensory epithelium, and most prominently in the growth zone (GZ) and at the periphery (P) of the sensory epithelium. (C) A merged image of FITC-phalloidin (A, green) and GTTR (B, red), showing the hair bundles super-imposed on GTTR-filled cell bodies (arrows). (D) At higher magnification, the peripheral red fluorescent cells in the growth zone have intense green fluorescent hair bundles (arrows) at their cell apices. Note negligible GTTR fluorescence in the extra-sensory epithelium (ES). (E) The red signal only from the image in (D) reveals negligible labeling of non-hair cells and diffuse fluorescence in hair cells. GTTR fluorescence within peripheral hair cells is both punctate and diffuse. (F) In the central region of the saccule, mature hair cells have large round apical surfaces, with an intensely fluorescent actiniferous hair bundle, and are surrounded by the green polygonal outlines of supporting cells. (G) The red signal only from the image in (F) reveals punctate GTTR labeling with diffuse GTTR fluorescence within the hair cell soma only. Scale bars in μm.

FIG. 13. GTTR and immunolabeled GT share similar distribution patterns in saccular explants incubated for 30 minutes (A,C) or 2 hours (B,D). (A) Explants incubated with 300 μm/ml GT/GTTR for 30 minutes display GTTR fluorescence throughout the sensory epithelium and preferentially at the periphery. (B) Explants incubated with 300 μm/ml GT/GTTR for 2 hours display less difference in the fluorescence between the peripheral and central zones. (C) Explants incubated with gentamicin for 30 minutes or (D) 2 hours, prior to gentamicin immunofluorescent labeling, reveal labeling in hair cells throughout the saccule, and somewhat preferentially at the periphery. (E) Explants incubated in normal culture media for 30 minutes, prior to fixation and then immunolabeled with gentamicin and secondary antibodies display negligible fluorescence. (F) Explants incubated with unconjugated GT for 2 hours, prior to fixation and immunolabeling with GT-adsorbed primary antibodies revealed negligible labeling. Scale bar in μm.

FIG. 14. GTTR and immunolabeled gentamicin are both localized in the hair bundle. In immature hair cells (A, B), phalloidin-Alexa-660-labeled hair bundles ( [symbol] ) appear above the sensory epithelium. GTTR (A′) and immunolabeled gentamicin (B′) fluorescence also occurs above the sensory epithelium in the region of the hair bundle ( [symbol] ). Co-localization analysis of single optical planes of explants double-labeled with phalloidin-Alexa-660 (blue) and GTTR (A″) or immunolabeled gentamicin (B″) reveal white pixels, indicating immature hair bundles ( [symbol] ) are co-labeled with GTTR or gentamicin antibodies. The kinocilium of several hair cells labeled with GTTR can also be seen (→) in A′ and A″. In the central saccule, mature hair cells labeled with phalloidin-Alexa-660-labeled hair bundles ( [symbol] ) appear above the sensory epithelium (C, D). GTTR (C′) or immunolabeled gentamicin (D′) fluorescence also occurs above the sensory epithelium in the region of the hair bundle ( [symbol] ), that can be verified as white pixels in the colorized images using co-localization analysis (C″, D″). The kinocilium of several mature hair bundles labeled with GTTR can also be seen (→) in C′ and C″. All images are from explants incubated with 300 μg/ml GT/GTTR or unconjugated GT (and subsequently immunolabeled) for 30 minutes. Scale bars in μm.

FIG. 15. GTTR and immunolabeled gentamicin are both localized in the nuclei of hair cells. In immature hair cells (A, B), Sytox Green-labeled nuclei appear at the periphery of the sensory epithelium. GTTR (A′) and immunolabeled gentamicin (B′) occurs in the same optical plane as Sytox Green-labeled nuclei. Co-localization analysis of single optical planes of nuclei double-labeled with Sytox Green and GTTR (A″) or immunolabeled gentamicin (B″) reveal white pixels, indicating nuclei that are co-labeled with GTTR or gentamicin antibodies. Only immunolabeled gentamicin (D″), but not GTTR (C″), can be readily seen in the nuclei of mature hair cells. GTTR (A′, A″) and immunolabeled gentamicin (B′, B″, D′, D″) is also present in the peri-nuclear cytoplasm (→). All images are from explants incubated with 300 μg/ml GT/GTTR or unconjugated GT (and subsequently immunolabeled) for 30 minutes. Scale bars in μm.

FIG. 16. (A-D) Explants preloaded with Lysotracker Green, Mitotracker Green, NBD-ceramide, and ERtracker that fluorescently label lysosomes, mitochondria, Golgi bodies and ER respectively, and subsequently incubated with GTTR for 2 hours. (A′-D′) Co-localization analysis reveals as white pixels those areas where the red and green fluorescence intensities are above a user-defined threshold, indicating that GTTR is co-localized in the region of fluorescently-labeled lysosomes (A′), mitochondria (B′), Golgi bodies (C′) and ER (D′). Scale bar in μm.

FIG. 17. Gentamicin immunoelectron microscopy of mature hair cells on LR Gold sections. (A) Immunogold labeling for gentamicin in a saccular hair cell is typically located in the vicinity of stereocilia (s), in the cuticular plate (cp), throughout the hair cell cytoplasm (see also inset), and often is associated with mitochondria (as in C, D). Note the comparative lack of labeling in the adjacent supporting cell (SC). (B) Anti-gentamicin immunogold labeling of sections cut from an explant incubated with normal culture media reveals negligible non-specific labeling. (C,D) Immunogold labeling for gentamicin is often associated with mitochondria ( [symbol] ) and (E,F) as clusters associated with electron dense inclusions within the cytoplasm. Scale bars in microns.

FIG. 18. Gentamicin immunoelectron microscopy. (A) Immunogold labeling ( [symbol] ) for gentamicin is also strongly associated with the nucleus of mature hair cells; inset (=Outlined area in main panel) shows strong labeling within the center of the nucleus. (B) Anti-gentamicin immunogold labeling sections cut from an explant not incubated with gentamicin reveals some non-specific labeling within the nucleus. Inset (=Outlined area in main panel) shows only few non-specific gold labeling within the center of the nucleus. (C) Immunogold labeling for gentamicin is also associated with the nucleus of immature hair cells (IM), with a comparative lack of labeling in adjacent supporting cell nucleus (SC). Inset (=Outlined area in main panel) shows strong gold labeling within the center of the immature hair cell nucleus compared to the supporting cell nucleus. Scale bars in microns.

FIG. 19. Bullfrog saccular immature hair cells accumulate less GTTR in vivo compared to explants in vitro.

FIG. 20. GTTR is more aggressively taken up at the base of the cochlea, as shown under low power.

FIG. 21. GTTR is more aggressively taken up at the base of the cochlea, as shown under high power.

FIGS. 22 and 26. GTTR uptakes are not replicated by free Texas Red.

FIG. 23. GTTR uptake is reduced by RTX, and by RTX plus Ca++. (A) At the level of the reticular lamina of the bullfrog saccule, bright GTTR fluorescence can be seen in hair cells (rounded apices), their hair bundles (arrows), and supporting cells (polygonal apices). (A′) GTTR uptake at the level of the reticular lamina is reduced by RTX, particularly in hair cells (rounded apices). (B) At the level of the hair cell nucleus, bright GTTR fluorescence is often found in the hair cell nuclei (arrowheads). (B′) RTX administration simultaneously with GTTR reduces the degree of GTTR uptake in the bullfrog saccule, and particularly in hair cell nuclei (arrowheads). (C) At the level of the reticular lamina of the bullfrog saccule, bright GTTR fluorescence can be seen in hair cells (rounded apices), their hair bundles (arrows), and supporting cells (polygonal apices). (C′) GTTR uptake at the level of the reticular lamina is reduced by co-treatment with RTX, and Ca++ particularly in hair cells (rounded apices). (D) At the level of the hair cell nucleus, bright GTTR fluorescence is often found in the hair cell nuclei (arrowheads). (D′) RTX and Ca++ administration simultaneously with GTTR reduces the degree of GTTR uptake in the bullfrog saccule, and particularly in hair cell nuclei (arrowheads).

FIG. 24. GTTR uptake in bullfrog saccular explants is reduced by RTX and Ruthenium Red, and increased by iodo-RTX. (A) Intense GTTR fluorescence in bullfrog saccular hair cells (rounded apices), hair bundles (arrows), and nuclei (arrowheads). (B) GTTR uptake in saccular hair cells is significantly reduced by RTX. (C) Intense GTTR fluorescence in bullfrog saccular hair cells (rounded apices), hair bundles (arrows), and nuclei (arrowheads). (D) GTTR uptake in saccular hair cells is significantly reduced by Ruthenium Red. (E) Intense GTTR fluorescence in saccular hair cells (rounded apices), hair bundles (arrows), and nuclei (arrowheads). (F) GTTR uptake in saccular hair cells is significantly enhanced by iodo-RTX. (G) Bright GTTR fluorescence in bullfrog saccular hair cells (rounded apices), hair bundles (arrows), and nuclei (arrowheads) after co-treatment with iodo-RTX. (H) GTTR uptake in saccular hair cells (co-treated with iodo-RTX) is significantly reduced by Ruthenium Red.

FIG. 25. RTX reduces uptake of GTTR in murine cochlear explants. (A) At the level of the reticular lamina of the organ of Corti saccule, bright GTTR fluorescence can be seen in hair cells apices (arrowheads), outer hair cell bodies, pillar cells (arrows). (B) GTTR uptake in the organ of Corti at the level of the OHC nuclei lamina is reduced by RTX, particularly in hair cells. (C) At the level of the reticular lamina, GTTR uptake is reduced by co-administration with RTX. (D) At the level of the OHC nucleus, GTTR uptake is reduced by co-administration with RTX. (E) GTTR uptake in the organ of Corti at the level of the OHC nuclei lamina is reduced further by RTX plus Ca++. (C) At the level of the reticular lamina, GTTR uptake is reduced by co-administration with RTX. (D) At the level of the OHC nucleus, GTTR uptake is reduced by co-administration with RTX, plus Ca++.

FIG. 27. Cytoplasmic and intra-nuclear binding of Texas Red-labeled gentamicin (GTTR). MDCK cells were treated with 1 μg/mL GTTR at 37° C. for 2 hours, washed and treated as described, then imaged using confocal microscopy. Control cells were treated identically with equivalent amounts of unconjugated, hydrolyzed Texas Red. All images were obtained using the same imaging parameters. A) Live cells with numerous GTTR-loaded vesicles. B) Cells washed after fixation with 4% formaldehyde containing 0.5% Triton X-100 (FATX) show fluorescent cytoplasmic and intra-nuclear labeling, but no punctate (vesicular) GTTR fluorescence. C) Cells in 4% formaldehyde alone (FA) show vesicular and surface labeling. D) Cells fixed with 4% FA and washed (as in C), then delipidated with 0.5% Triton X-100 and washed show bright cytoplasmic and intra-nuclear labeling, as in B). Arrows show fluorescent structures traversing the nucleus. E) Cells fixed with FATX (as in B) treated with 1 mg/mL PIP2 for 1.5 hours show quenched fluorescence. F) Cells treated with PIP2 (as in E) then delipidated with 0.5 Triton X-100 and rinsed show recovery of fluorescence brightness. G) Live cells treated with TR alone and imaged have TR-loaded vesicles. H) Cells treated with TR, then fixed in FATX buffer and washed have no cytoplasmic or nuclear fluorescence.

FIG. 28. Influence of time and temperature on GTTR binding. OK cells were treated with 1 μg/mL GTTR at 37° C. or on ice and imaged live (insets) or fixed after specified time intervals. A1-A6) Cells at 37° C. show increasing cytoplasmic GTTR binding over time. Insets show cells imaged live, and endocytotic uptake 15 minutes (or later) after GTTR application. B1-B6) Cells held over ice during treatment show increased GTTR binding over time, but less intensely than that seen at 37° C. Scale bar=20 μm. Color gradient at base represents fluorescent intensity from the hot.lut lookup table: 0 (no fluorescence, black) to 255 (saturated pixels, white), and enhances the ability of the human eye to discriminate intensity differences over grayscale images. Note live images in insets were acquired without washing out GTTR from the extracellular medium, so fluorescence is visible outside the cells.

FIG. 29. Morphology of OK and MDCK cells in complete medium with or without streptomycin. A). At low density, OK cells raised in penicillin-streptomycin culture media (OKps) look fibroblastic. B) At high density, OKps cells retain a fibroblastic morphology and crowd together (*). C) At low density, OK cells, grown in streptomycin-free media (OKsf) for at least 7 weeks, cluster together and have an epitheloid appearance. D) At high density, confluent OKsf cells retain their epitheloid appearance. E) At low density, MDCK cells, grown in streptomycin-free media for at least 7 weeks (MDCKsf), cluster together and appear epitheloid (as in penicillin-streptomycin media). F) At high density, confluent MDCKsf cells retain their epitheloid appearance. Scale bar=50 μm.

FIG. 30. PIP₂ quenching of GTTR but not TR fluorescence. A-D) 3-dimensional excitation and emission scans; excitation 570-640 nm, emission 610-650 nm, bandwidth=5 nm, emission intensity in arbitrary fluorescent units. A) Texas Red. B) Texas Red in the presence of PIP₂. Note the similarity of the spectra to that seen in A). C) GTTR. D) GTTR in the presence of PIP₂. Note the reduced fluorescence emission intensities compared to C). E) Emission scan of GTTR at 618 nm (bandwidth=5 nm) in the absence (red) or presence (blue) of PIP₂ over the excitation wavelength range 290-604 nm. F) Emission scan of GTTR at the fixed excitation wavelength 587 nm (bandwidth=5 nm) over the emission wavelength range 598-748 nm (bandwidth=5 nm) in the absence (red) or presence (blue) of PIP₂.

FIG. 31. Distribution of GTTR in methanol-fixed MDCK cells double-labeled with Syto RNASelect. A) GTTR is diffusely distributed throughout the cytoplasm, and strongly labels the intra-nuclear structures (arrows), and trans-nuclear tubules (double arrowhead in inset). B) Syto RNASelect strongly labels the globular intra-nuclear structures (arrows), and trans-nuclear tubules (double arrowhead in inset). C) Merged images of (A) and (B), show co-localization of both GTTR and SYTO RNASelect fluorophores as yellow in globular intra-nuclear structures (arrows), and trans-nuclear tubules (double arrowhead in inset). D) Fluorescent GTTR-loaded cells. E) Cells in (D), not treated with Syto RNASelect, display negligible non-specific 515 nm fluorescence. F) Merged image of (D) and (E). G) Cells treated with SytoRNASelect only (in H) display no bleed-through fluorescence in red (GTTR) channel. H) Cells treated with Syto RNASelect. Note mitotic figure lower left. I) Merged image of (G) and (H). Scale bars=10 μm.

FIG. 32. Saturability of cytoplasmic, but not vesicular GTTR fluorescence. OK or MDCK cells were treated with 1 μg/mL GTTR for 120 minutes at 37° C. or held over ice in the presence of increasing concentrations of unlabeled gentamicin. Cells were imaged live or fixed with FATX and washed before imaging. A1-A5) At 37° C., OK cells, treated with GTTR show serially reduced labeling with increasing unlabeled GT concentrations. Inset in A1) shows only vesicular GTTR labeling in live OK cells treated with GTTR alone. Inset in A5) also shows little or no decrease in vesicular GTTR labeling in live OK cells treated with GTTR plus 4 mg/mL unlabeled gentamicin. B1-B5) OK cells on ice show reduced GTTR binding in the cytoplasm and intra-nuclear compartments (compared to 37° C., A1-A5) and reduction of binding with increasing concentrations of unlabeled GT concentrations. Inset in B1) shows no vesicular GTTR uptake in OK cells on ice when imaged live. C1-C5) MDCK cells on ice also show decreasing cytoplasmic GTTR fluorescence as concentration of unlabeled gentamicin increases.

FIG. 33. Immunocytochemical localization of GTTR and unlabeled gentamicin. MDCK cells were treated with 5 μg/mL GTTR (A-F), or 300 μg/mL unlabeled gentamicin (G,H) for 2 hours, fixed with FA only, permeabilized with methanol, and immunolabeled. A) In cells incubated at 37° C., GTTR fluorescence occurs as both diffuse and punctate (arrowheads) cytoplasmic labeling, with labeled nucleoli (arrows, and inset) and trans-nuclear tubules (double arrowheads, inset). B) Gentamicin immunolabeling of GTTR seen in (A) also reveals diffuse cytoplasmic, and intracellular puncta (arrowheads) of labeling; with weak labeling of the nucleoplasm, and labeled trans-nuclear tubules (double arrowheads, inset). GTTR-labeled nucleoli are not immunolabeled (arrows). C) Merged images of (A) and (B), show co-localization of both GTTR and immunofluorescence as yellow in the cytoplasm, puncta (arrowheads), and intra-nuclear tubules (double arrowheads). D) Cells loaded with GTTR on ice reveal very few intracellular puncta of fluorescence (compared to A), and robust, diffuse cytoplasmic and weak nucleoplasmic labeling, with labeled trans-nuclear tubules (double arrowheads) and nucleoli (arrows, inset). E) Gentamicin immunolabeling of GTTR seen in (A) also reveals only diffuse cytoplasmic and nucleoplasmic labeling, with weakly labeled trans-nuclear tubules (double arrowheads, inset). GTTR-labeled nucleoli are not immunolabeled (arrows). F) Merged image of (D) and (E). G) Immunofluorescence of GT-loaded cells incubated at 37° C. ice reveals diffuse cytoplasmic and nucleoplasmic labeling, intracellular fluorescent puncta (arrowheads). Presumptive sites of nucleoli are not immunolabeled (arrows). H) Immunofluorescence of GT-loaded cells incubated on ice reveals diffuse cytoplasmic and nucleoplasmic labeling, with few intracellular puncta of fluorescence. Presumptive GTTR-labeled intra-nuclear structures are not immunolabeled (arrows). I) Cells, without GT or GTTR treatment, incubated on ice and immunoprocessed with primary and secondary IgG display negligible non-specific labeling. Scale bars=10 μm.

FIG. 34: Increasing Kₒ reduces GTTR uptake. MDCK cells were washed twice with HBSS then treated with 1 μg/mL GTTR for 1 minute at 20° C., washed twice with HBSS, then fixed with FATX and washed. HBSS was mixed with equi-osmolar KCl/HBSS to produce the required K⁺ concentrations. A) 5.8 mM K⁺; B) 10 mM K⁺; C) 40 mM K⁺; D) 140 mM K⁺. Scale bar=10 μm.

FIG. 35: Lanthanum and Gadolinium block cation channels. FIG. 35A1, GTTR uptake in absence of Gd⁺⁺⁺; FIG. 35 C2-C3, GTTR uptake in presence of Gd⁺⁺⁺; FIG. 35 B1, GTTR uptake in absence of La⁺⁺⁺; FIG. 35 B2-B4, GTTR uptake in presence of La⁺⁺⁺; FIG. 35 C1, gentamicin immuno labeling in FATX-fixed cells in the absence of La⁺⁺⁺; and FIG. 35 C2-C4, in presence of La⁺⁺⁺.

FIG. 36: Modulation of GTTR uptake into cytoplasmic and intra-nuclear compartments by calcium, pH TRPV1 agonists and antagonists. In all images, MDCK cells were treated as indicated for 30 seconds with 5 μg/mL GTTR at 20° C., washed, fixed with FATX and washed again. All images were obtained using the same imaging parameters. A1-A7) Cells treated with varying Ca⁺⁺ concentrations show increased binding from 0 to 0.16 mM Ca⁺⁺ with serially declining binding at higher concentrations. B1-B7) Cells were treated in PBS at varying pH, as indicated, show maximal GTTR binding at pH 5 (B2), with significantly decreased binding in more basic buffers (B3-B6), and greatly reduced GTTR uptake at pH 4 (B1) and pH 10 (B7). C1 and D1) GTTR alone. C2-4) Cells treated with TRPV1 agonist RTX show stimulation of GTTR uptake at 5×10⁻⁹ M, with declining stimulation at higher RTX concentrations; C5-7) Cells treated with TRPV1 agonist anandamide also show stimulation of GTTR uptake at 10⁻⁶ M and 10⁻⁵ M with little or no stimulation at 10⁻⁴ M. D2-4) Cells treated with antagonist SB366791 show increasing stimulation of GTTR uptake at increasing concentrations (10⁻⁷ to 10⁻⁵M). D5-7) Cells treated with antagonist iodo-RTX also show increasing stimulation of GTTR uptake at increasing concentrations (10⁻⁷ to 10⁻⁵ M). E1-5) Cells treated with GTTR and 100 μM Ruthenium Red alone, or with agonists or antagonists at their most effective tested doses. E1) RR alone shows decreased GTTR uptake compared to D1. E2-5) RR blocked enhanced GTTR uptake induced by all agonists and antagonists tested, with only a partial effect on anandamide stimulation (E4). F1) No fluorescence is present in the cytoplasmic compartment when hydrolyzed TR is added with 10⁻⁵ M I-RTX. F2) No fluorescence occurs in the cytoplasmic compartment when hydrolyzed TR is added with 5×10⁻⁷ M RTX. Scale bar in D7=20 μm, and applies to all image panels.

FIG. 37: Calcium attenuation of RTX effect. (A-C) MDCK cells were treated for 30 seconds at room temperature at pH 7.0 with 1 μg/mL of GTTR in 138 mM saline (A1, B1, C1), or (A2, B2, C2,) saline with 0.16 mM or (A3, B3, C3) 2.0 mM calcium. In each of these solutions, cells received no other treatment, or 5×10⁻⁹ M RTX, or 10⁻⁵ M I-RTX. A1) no calcium, no other treatment; A2) 0.16 mM calcium, no other treatment; A3) 2.0 mM calcium, no other treatment; B1) no calcium, RTX; B2) 0.16 mM calcium, RTX; B3) 2.0 mM calcium, RTX; C1) no calcium, I-RTX; C2) 0.16 mM calcium, I-RTX; C3) 2.0 mM calcium, I-RTX. Scale bar in I=20 μm applies to all image panels.

FIG. 38: FIG. 38 A, MDCK cells treated with K⁺ and GTTR; FIG. 38 B-D, cells treated with increasing concentrations of K⁺, showing GTTR uptake; FIG. 38E, F, cells treated with valinomycin, showing effect on GTTR uptake compared to control cells.

### DETAILED DESCRIPTION OF THE INVENTION

The present invention approaches the problem of antibiotic toxicity from the unique vantage point of preventing uptake of the drug by the cell in the first place, thus circumventing the need to modulate any type of apoptotic or necrotic mechanism. By preventing drug uptake by the cells of the inner ear and the kidney, but allowing administration of the drug to the site of infection, this invention allows the therapeutic function of the drug to be utilized more effectively and for a wider range of illnesses than is currently possible. The invention allows use of drugs that are too toxic, and that currently are only administered topically, to be administered by other routes.

As will be disclosed in further detail below, gentamicin enters cells via non-endocytotic mechanisms, probably via TRPV-1 channels, and likely also through other TRP channels. The uptake is regulatable, and regulation of uptake should alter its toxicity. The invention provides methods for measuring uptake, toxicity, and various metabolic responses to compare acute effects to toxicity. These methods can be applied directly or indirectly to other aminoglycosides, and to other therapeutics that share specific chemical characteristics of aminoglycosides. The present data show that the bio-relevant uptake of gentamicin, and a large group of similar agents, is mediated by a family of non-specific calcium permeant cation channels (TRPs). This has allowed the inventors to identify mechanisms which can be exploited to block drug penetration into cells, and thus also the passage of drug across epithelial and endothelial layers (i.e., transcytosis, which involves penetration into cells). Preventing (or reducing) penetration of toxic drugs into cells is far more efficacious than trying to offset harmful effects after drugs have reached their targets.

The invention relates to therapies, including the use of cation channel regulating blockers or drugs to reduce cellular uptake and epithelial/endothelial transcytosis of oto- and nephrotoxic agents, which have the characteristic of being polycationic at physiological pH. These include, but are not limited to, aminoglycosides, cisplatinum and cephalosporins.

The invention also relates to methods for the synthesis and isolation of bioactive GTTR, GTTR uptake assays, toxicity assays, and metabolic assays.

Definitions

As used herein, the term “vertebrate” has its customary meaning including any backboned animal including domestic, farm, pet, and zoo animals.

As used herein, “mammal” for purposes of treatment refers to any animal classified as a mammal, including humans, domestic, and farm animals, and zoo, sports, or pet animals, such as dogs, horses, cats, sheep, pigs, cows, etc. The preferred mammal herein is a human.

“Treatment” refers to both therapeutic treatment and prophylactic or preventative measures, wherein the object is to prevent or slow (lessen) inner ear tissue-damage-related hearing disorder or impairment or to prevent or slow (lessen) kidney tissue-damage-related renal function disorder or impairment. Those in need of treatment include those already experiencing a hearing or kidney impairment, those prone to having a hearing or kidney impairment, and most preferably those in which the potential impairments are to be prevented.

The hearing impairments are due to inner ear hair cell damage or loss, wherein the damage or loss is caused by infection, mechanical injury, aging, or, preferably, chemical-induced ototoxicity, wherein ototoxins include therapeutic drugs including antineoplastic agents, salicylates, quinines, diuretics including furosemide, ethocrynic acid, and aminoglycoside and polypeptide antibiotics, contaminants in foods or medicinals, and environmental or industrial pollutants, solvents, including toluene, xylene, etc., in view of the known risk of deafness in paint sprayers due to occupational exposure; metalloproteins, including arsenic, cadmium, etc., and iron. The common factor is structural similarity to aminoglycoside antibiotics to the extent that the structural similarity allows use of the mechanism described herein for entry of the structure via the relevant cation channels. Typically, treatment is performed to prevent or to reduce ototoxicity, especially resulting from or expected to result from administration of therapeutic drugs. Preferably a therapeutically effective composition is given immediately after the exposure to prevent or reduce the ototoxic effect. More preferably, treatment is provided prophylactically, either by administration of the composition prior to or concomitantly with the ototoxic pharmaceutical or the exposure to the ototoxin.

Impairments of kidney function are due to cell damage or loss within the proximal or distal tubules of the kidney, wherein the damage or loss is caused by infection, mechanical injury, aging, or, preferably, chemical-induced nephrotoxicity, wherein nephrotoxins include therapeutic drugs including aminoglycoside antibiotics, contaminants in foods or medicinals, and industrial pollutants, including the compounds described above in reference to hearing impairment. Typically, treatment is performed to prevent or to reduce ototoxicity, especially resulting from or expected to result from administration of therapeutic drugs. Preferably a therapeutically effective composition is given immediately after the exposure to prevent or reduce the nephrotoxic effect. More preferably, treatment is provided prophylactically, either by administration of the composition prior to or concomitantly with the nephrotoxic pharmaceutical or the exposure to the nephrotoxin.

By “ototoxic agent” in the context of the present invention is meant a substance that through its chemical action injures, impairs, or inhibits the activity of a cell or tissue component related to hearing, which in turn impairs hearing and/or balance. In the context of the present invention, ototoxicity includes a deleterious effect on the inner ear sensory hair cells. Ototoxic agents that cause hearing impairments include, but are not limited to, neoplastic agents such as vincristine, vinblastine, cisplatin, taxol, or dideoxy-compounds, e.g., dideoxyinosine; alcohol; metals (iron, arsenic, mercury); industrial toxins involved in occupational or environmental exposure (toluene, xylene); contaminants of food or medicinals; or large doses of vitamins or therapeutic drugs, e.g., antibiotics such as penicillin or chloramphenicol, or megadoses of vitamins A, D, or B6, salicylates, quinines and loop diuretics. Other toxic agents that can cause ototoxicity-inducing hearing impairment can be identified and characterized by methods as taught herein. By “exposure to an ototoxic agent” is meant that the ototoxic agent is made available to, or comes into contact with, a vertebrate, such as a mammal. Exposure to an ototoxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

By “nephrotoxic agent” in the context of the present invention is meant a substance that through its chemical action injures, impairs, or inhibits the activity of a component of the renal system, which in turn impairs the function of the kidney. In the context of the present invention, nephrotoxicity includes a deleterious effect on the cells of the kidney, particularly the cells of the proximal and distal tubules. Nephrotoxic agents that cause impairments of kidney function include, but are not limited to, those discussed above in reference to hearing impairment. Other toxic agents that can cause nephrotoxicity-inducing impairment of kidney function can be identified and characterized by methods as taught herein. By “exposure to a nephrotoxic agent” is meant that the nephrotoxic agent is made available to, or comes into contact with, a vertebrate, such as a mammal. Exposure to a nephrotoxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

By “TRP” is meant Transient Receptor Potential; a TRP agonist induces a short-lived cation current in cells expressing TRP specific for agonist. Four major families of TRV have been described: TRPC, TRPV, TRPM, PKD. The receptors appear to be mainly sensory: sight, hearing, chemosensory, osmo-regulatory, taste, temperature, and mechanosensory. TRPs are largely non-voltage gated, calcium-permeant, cation channels, and the family is largely responsible for calcium homeostasis in non-electrically active cells.

TRPs are also called “capacitative calcium entry” channels, which are channels that respond to depletion of intracellular calcium stores by opening to permit calcium entry. (Calcium entry produces a “current” and intracellular stores act as “capacitors.”) Thus, cations enter through these channels in the absence of their named ligands.

One particular TRP referred to in the context of this invention is TRPV1, which senses heat and capsaicin (chili pepper)-like molecules. TRPV1 is also called the vanilloid receptor.

Modes for Carrying Out the Invention

In the human population, patients targeted for treatment by the current invention include those patients who are subject to hearing and/or renal impairment that would be otherwise caused by ototoxic or nephrotoxic drugs that affect inner ear hair cells and/or cells of the proximal or distal tubules of the kidney. These patients include those diagnosed with tuberculosis, cystic fibrosis, meningitis, plague and burns; patients given rabies prophylaxis; and surgical patients; and others who may be given antibiotics or other treatments as described herein.

Hearing impairments relevant to the invention are preferably sensory hearing loss due to end-organ lesions involving inner ear hair cells, such as, viral endolymphatic labyrinthitis, and Meniere's disease. Hearing impairments include tinnitus, which is a perception of sound in the absence of an acoustic stimulus, and may be intermittent or continuous, wherein there is diagnosed a sensorineural loss. Hearing loss may be due to bacterial or viral infection, such as in herpes zoster oticus, purulent labyrinthitis arising from acute otitis media, purulent meningitis, chronic otitis media, sudden deafness including that of viral origin, e.g., viral endolymphatic labyrinthitis caused by viruses including mumps, measles, influenza, chickenpox, mononucleosis and adenoviruses. The hearing loss can be congenital, such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome.

The hearing loss may be caused by an ototoxic drug that affects the auditory portion of the inner ear, particularly inner ear hair cells. Incorporated herein by reference are Chapters 196, 197, 198 and 199 of the Merck Manual of Diagnosis and Therapy, 14^{th} Edition, (1982), Merck Sharp & Dome Research Laboratories, N.J. and corresponding chapters in the most recent 16^{th} edition, including Chapters 207 and 210, relating to description and diagnosis of hearing and balance impairments.

Tests are known and available for diagnosing hearing impairments. Neuro-otological, neuro-ophthalmological, neurological examinations, and electro-oculography can be used. (Wennmo et al. Acta Otolaryngol (1982) 94:507-15). Sensitive and specific measures are available to identify patients with auditory impairments. For example, tuning fork tests can be used to differentiate a conductive from a sensorineural hearing loss and determine whether the loss is unilateral. An audiometer is used to quantify hearing loss, measured in decibels. With this device the hearing for each ear is measured, typically from 125 to 8000 Hz, and plotted. The speech recognition threshold, the intensity at which speech is recognized as a meaningful symbol, can be determined at various speech frequencies. Speech or phoneme discrimination can also be determined and used as an indicator of sensorineural hearing loss since analysis of speech sounds relies upon the inner ear and the 8^{th} nerve. Tympanometry can be used to diagnose conductive hearing loss and aid in the diagnosis of those patients with sensorineural hearing loss. Electrocochleography, measuring the cochlear microphonic response and action potential of the 8^{th} nerve, and evoked response audiometry, measured evoked response from the brainstem and auditory cortex, to acoustic stimuli can be used in patients, particularly infants and children or patients with sensorineural hearing loss of obscure etiology. These tests serve a diagnostic function as well as a clinical function in assessing response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including neural hearing loss. In sensory hearing loss, the sensation of loudness in the affected ear increases more with each increment in intensity than it does in the normal ear. Sensitivity to small increments in intensity can be demonstrated by presenting a continuous tone of 20 dB above the hearing threshold and increasing the intensity by 1 dB briefly and intermittently. The percentage of small increments detected yields the “short increment sensitivity index” value. High values, 80 to 100%, are characteristic of sensory hearing loss, whereas a neural lesion patient and those with normal hearing cannot detect such small changes in intensity. Pathologic adaptation is demonstrated when a patient cannot continue to perceive a constant tone above threshold of hearing, also known as tone decay. A Bekesy automatic audiometer or equivalent can be used to determine these clinical and diagnostic signs; audiogram patterns of the Type II pattern, Type III pattern and Type IV pattern are indicative of preferred hearing losses suitable for the treatment methods of the invention. As hearing loss can often be accompanied by vestibular impairment, vestibular function can be tested, particularly when presented with a sensorineural hearing loss of unknown etiology.

When possible, diagnostics for hearing loss, such as audiometric tests, should be performed prior to exposure in order to obtain a patient's normal hearing baseline. Upon exposure, particularly to an ototoxic drug, audiometric tests should be performed twice a week and testing should be continued for a period after cessation of the ototoxic drug treatment, since hearing loss may not occur until several days after cessation. U.S. Pat. No. 5,546,956 provides methods for testing hearing that can be used to diagnose the patient and monitor treatment. U.S. Pat. No. 4,637,402 provides a method for quantitatively measuring a hearing defect that can be used to diagnose the patient and monitor treatment.

Hearing impairments and impairments of the kidney that are induced by aminoglycosides can be prevented or reduced by the methods of the invention. Although the aminoglycosides are useful therapeutic agents for the treatment of infections due to their rapid bactericidal action, their use is currently limited to severe or complex infections due to their severe ototoxic and nephrotoxic side effects.

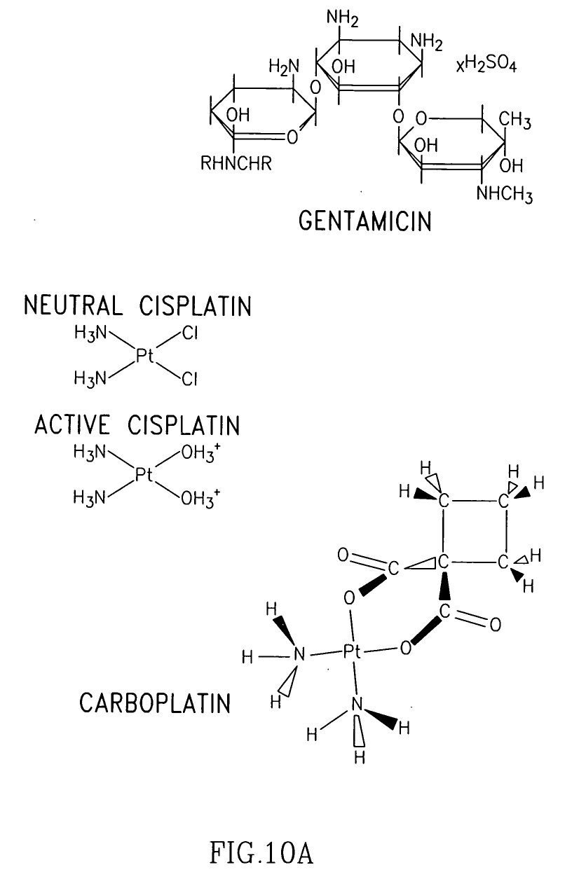

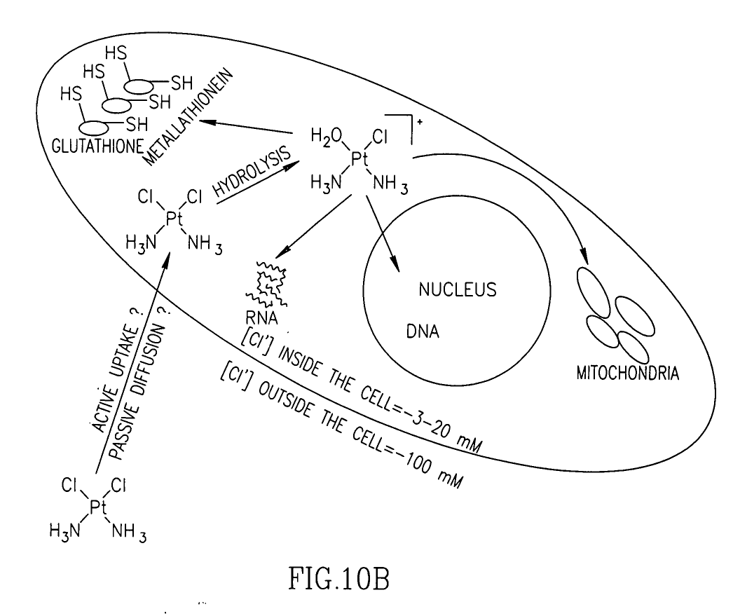

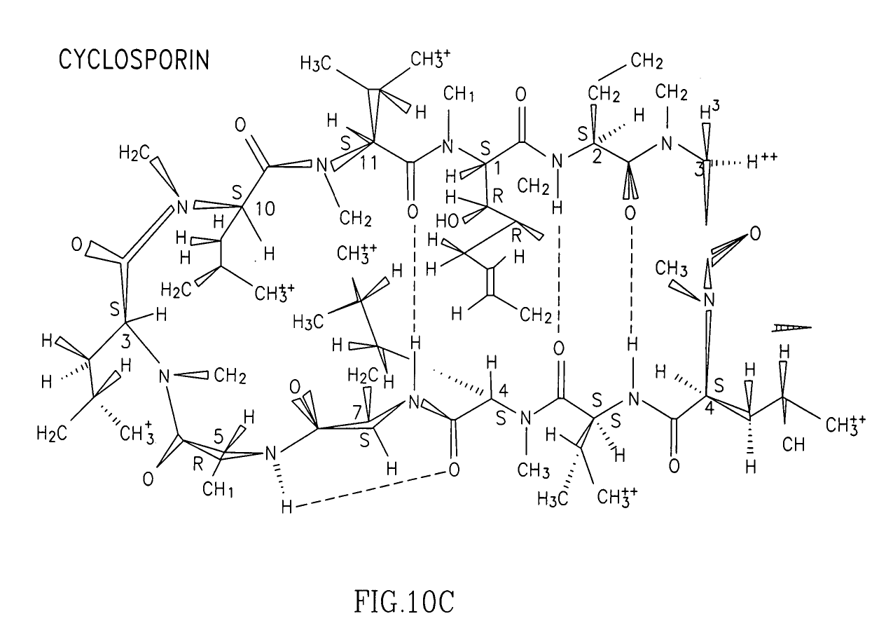

Aminoglycosides belong to a class of compounds characterized by the ability to interfere with protein synthesis in microorganisms. Aminoglycosides consist of two or more amino sugars joined in a glycoside linkage to a hexose (or aminocyclitol) nucleus. The hexose nuclei thus far known are either streptidine or 2-deoxystreptamine, though others may be identified and are within the scope of the invention. Aminoglycoside families are distinguished by the amino sugar attached to the aminocyclitol. For example, the neomycin family comprises three amino sugars attached to the central 2-deoxystreptamine. The kanamycin and glutamicin families have two amino sugars attached to the aminocyclitol. Aminoglycosides include neomycin, paromomycin, ribostamycin, lividomycin, kanamycins, amikacin, tobramycin, viomycin, gentamicin, sisomicin, netilmicin, streptomicin, dibekacin, fortimicin, and dihydrostreptomycin. Any of these aminoglycosides can be employed in conjunction with the present invention to prevent the ototoxic and nephrotoxic side effects of therapeutically effective amounts of the aminoglycosides. Aminoglycoside chemical structures are shown in FIG. 10.

The accumulation of aminoglycoside antibiotics by a variety of subcellular organelles suggests a variety of interactions between aminoglycosides and eukaryotic cells, ranging from interactions with ion channels/receptors and endocytotic uptake, to modulating intracellular chemical activities.

Current attempts to ameliorate ototoxicity have used cell death inhibitors in a cocktail with the aminoglycoside antibiotics. These cell death inhibitors include anti-oxidants and salicylate (Sha and Schacht, Lab. Invest. 79:807-813; Hear. Res. 142:34-49, 1999), inhibitors of caspase-3 (Liu et al., 1998), c-Jun kinase (Ylikosi et al., 2002) and calpain (Ding et al., 2002). It is believed that these agents produce their effects after the ototoxic drug has already been taken up by the cell. There are many problems with the current approaches to ameliorating the toxic side effects of aminoglycoside antibiotics. First, before the cell death mechanism is halted, the aminoglycoside has entered the cell and is able to exert its toxic effects on the cell. Even though the cell death inhibitors will arrest the progression to apoptosis, there are still numerous harms that can be exerted against the toxicated cell short of death, including loss of sensory function, which can nonetheless harm and disable the patient even without actual cell death.

A second drawback to current approaches to reduce cell death due to aminoglycoside antibiotics after the antibiotics have already entered the cell is that cell death inhibitors only arrest cell death mechanisms—they do not completely halt the process altogether. Thus, if inhibition is removed, progress along the cell death pathway could continue.

The novel methods of the present invention are designed to prevent cell death by preventing drug uptake by the cells themselves. At the same time, preventing the drugs from entering the mammalian cells will not reduce their efficacy as antibiotics, as the mechanisms by which these drugs act as bactericidal agents are distinct from the mechanisms by which these drugs are taken up by human cells. Preventing entry to the human cells should thus have little or no significant effect on the ability of these drugs to treat bacterial infections.

The present invention therefore relates to blocking drug penetration into sensory hair cells and the cells of the kidney. Preventing or reducing penetration of toxic drugs into cells is more efficacious than trying to offset harmful effects after drugs have already reached the cytoplasm or nuclei of the mammalian cells.

The methods of the invention are particularly effective when the toxic compound is an antibiotic, preferably an aminoglycoside antibiotic. Such aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include but are not limited to neomycin B, kanamycin A, kanamycin B, gentamicin C1, gentamicin C1a, and gentamicin C2. Prior to this invention, aminoglycosides were believed to enter hair cells via endocytotic mechanisms. There is circumstantial evidence that aminoglycosides may also enter hair cells through the unidentified mechano-electrical transduction (Gale et al., 2001), as well as blocking that channel (Ricci, J. Neurophysiol. 87: 1738-1748, 2002; Kroese, et al., Hear. Res. 37:203-217, 1989) through unidentified cation channels, including the mechansensory transduction channel of sensory hair cells (Gale et al. 2001).

TRP channel proteins constitute a large and diverse family of proteins that are expressed in many tissues and cell types. (Minke and Cook, Physiol Rev. 2002. 82:429-472). This family was designated TRP because of a spontaneously occurring Drosophila mutant lacking TRP that responded to a continuous light with a transient receptor potential (TRP). In addition to responses to light, TRPs mediate responses to nerve growth factor, pheromones, olfaction, mechanical, chemical, temperature, pH, osmolarity, vasorelaxation of blood vessels, and metabolic stress. TRP channels readily allow permeation by a variety of monovalent ions including Na, K, Cs, Li, and even large organic cations such as Tris and TEA (Hardie, Proc R Soc Lond B Biol Sci 245: 203-10; Ranganathan et al., Nature 1991 354: 230-32). The TRPV subfamily includes the mammalian vanilloid receptor, which has been found to mediate the pain pathway (Caterina et al., Nature 1997 389:816-24).

Without being bound by a specific mechanism, the present invention is based on the premise that by blocking access of aminoglycosides to TRP channels, accumulation of these drugs within the cytoplasm of hair cells and/or kidney cells can be prevented or reduced.

In one embodiment of the invention, cocktails of blockers are used to prevent oto- and nephrotoxicity. These are mixtures of TRP-specific blocking agents, as well as non-specific agents such as divalent cations (e.g. Ca, Mg, Zn), and partially permeant peptide constructs.

The oto- and nephro-protective agents are directly administered to the patient by any suitable technique, including parenterally, intranasally, intrapulmonary, orally, or by absorption through the skin. If the protective agents are administered concomitantly with the ototoxic or nephrotoxic agent, the protective agent does not have to be administered by the same route as the toxic agent. Protective agents can be administered locally or systemically. Examples of parenteral administration include subcutaneous, intramuscular, intravenous, intra-arterial, and intra-peritoneal administration. They can be administered by daily subcutaneous injection. They can also be administered by implants. The specific route of administration will depend, for example, on the medical history of the patient, including any perceived or anticipated side effects using the protective agent alone, and the particular disorder to be corrected.

Delivery of therapeutic agents in a controlled and effective manner with respect to tissue structures of the inner ear, for example, those portions of the ear contained within the temporal bone which is the most dense bone tissue in the human body, is known in the art. Exemplary inner ear tissue structures of primary importance include but are not limited to the cochlea, the endolymphatic sac/duct, the vestibular labyrinth, and all of the compartments which include these components. Access to the foregoing inner ear tissue regions is typically achieved through a variety of structures, including but not limited to the round window membrane, oval window/stapes footplate, the annular ligament, and systemically.

The design and synthesis of blockers according to the invention can be carried out using a variety of methods including molecular modeling based on the core structure of the aminoglycoside antibiotics. Proof of principle can be carried out routinely using gentamicin and the in vitro and in vivo models described herein and known in the art. The novel aspect is the disclosure for the first time that the blockers should specifically interfere with and preferably prevent entry of the aminoglycoside-like structure to the cell via a TRP channel, preferably a TRPV1 channel.

Methods of designing candidate blockers include those described in Honma, T., Medicinal Research Reviews 23:606-632, 2003, incorporated by reference. For example, validation of small fragments, which are substructures of ligands or blockers, can be performed by NMR, X-ray and Mass Spec. This method provides an alternative to bioassay of numerous candidate blockers. In one scheme, de novo design is carried out to design structures that mimic the structural interaction of an aminoglycoside and a TRPV1 channel. The structures (candidate blockers) are evaluated on the basis of chemical availability, synthesis of derivatives, assays, and validation by X-ray, NMR, and MS. The blockers are then synthesized and assayed, and a 3D library is designed based on successful candidates. As the structures of compounds intended to be blocked are known, the goal for the candidate blockers is also known: prevention of aminoglycoside entry via TRP channels, whether through steric hindrance or other physical or chemical means.

The following examples are offered by way of illustration and not by way of limitation.

### EXAMPLE 1

### TRPV1 Mediates Gentamycin Entry in Cultured Kidney Cells

According to this example, an endosome-independent mechanism by which gentamicin crosses the plasma membrane directly into the cytoplasm and then into intra-nuclear compartments was characterized and validated. The fluorescence of GTTR in these compartments was quenched by cellular lipids. The results also show that the vanilloid receptor, TRPV1, is involved in the uptake of Texas Red-labeled gentamicin into the kidney distal tubule cell line MDCK.

Regulation of GTTR Uptake:

MDCK cells were used as a model system to test regulation of GTTR uptake by conditions known to produce or modify a cation current through the TRPV1 channel. Conditions tested were varying extracellular calcium concentrations, pH, specific agonists, specific antagonists, and the non-specific cation channel blocker Ruthenium Red. In addition, we pre-mixed PIP₂ with GTTR to determine whether that anionic phospholipid, known to bind gentamicin, would alter GTTR uptake. All these assays were done at room temperature (20° C.), for 30 or 60 seconds, and using doses of GTTR of 5 to 10 μg/ml, which are far below the typical therapeutic level of <300 μg/ml. Buffers used for treatment were as described for each experiment. Cells were quickly washed to replace culture medium with treatment buffer, treated as indicated, washed again after treatment, fixed immediately with 4% formaldehyde and 0.5% Triton X-100 (FATX), and washed prior to imaging.

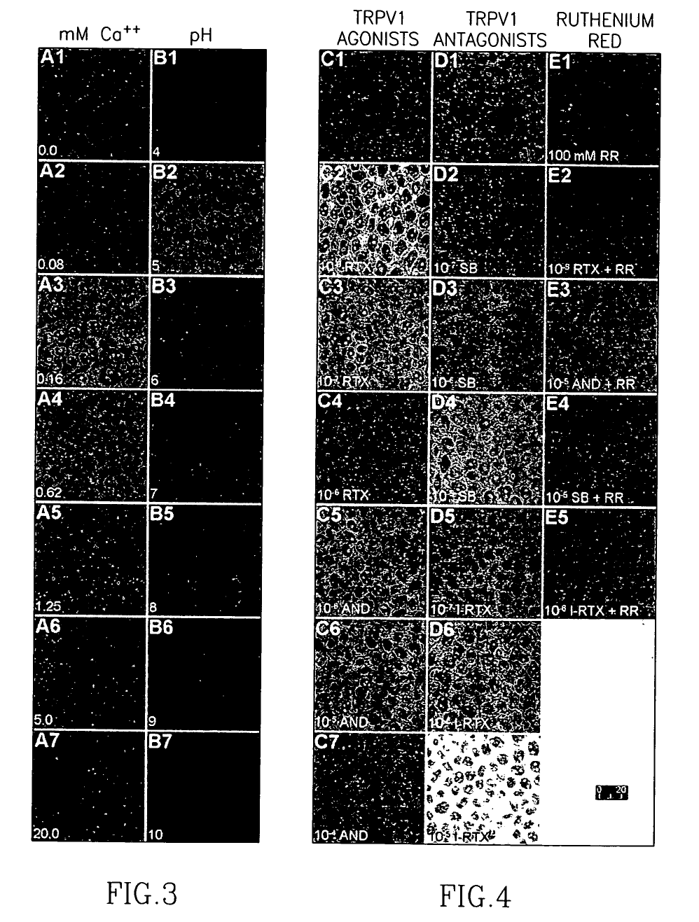

Calcium: Changes in extracellular calcium altered GTTR uptake. Cells were quickly washed twice with 0.9% saline then treated with GTTR in saline at the indicated concentration of calcium chloride, at pH 7.3. When no calcium was added, GTTR uptake was low (FIG. 3, A1), but increased at 0.16 mM calcium (FIG. 3, A3). As calcium concentrations increased above 0.16 mM, GTTR uptake decreased (FIG. 3, A4-A7). Those data are consistent with gentamicin penetration of cation channels.

Protons: Changes in extracellular pH altered GTTR uptake. Cells were washed with saline and treated with GTTR in buffer at pH ranging from 4 to 10. These experiments were performed in three different buffers, PBS (no calcium), a mixture of one part PBS and one part HBSS for a final calcium concentration of 0.63 mM, and a mixture of three parts PBS to one part HBSS for a final calcium concentration of 0.315 mM. In all cases the effect of pH was the same and only the PBS data are shown. At pH 5, and to a lesser extent at pH 6, uptake of GTTR increased (FIG. 3, B2 and B3), consistent with the reported pH range of proton stimulation of inward current through the TRPV1 channel. At pH 4, uptake was lower (FIG. 3, B1). Increasingly basic conditions reduced uptake (FIG. 3, B4-B7). The effects of both calcium and protons on GTTR uptake are consistent with the possibility that TRPV1 channels can play a role in the penetration of gentamicin into the cytoplasm of kidney cells.

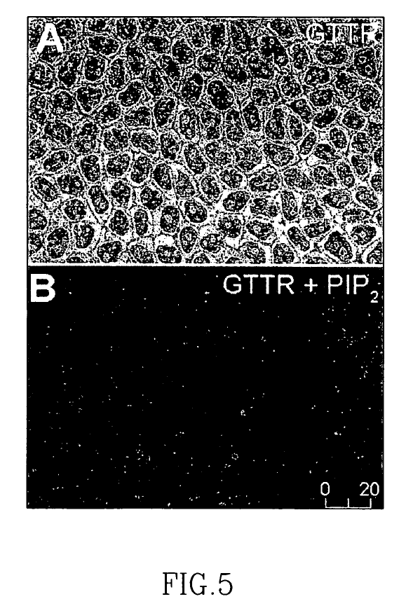

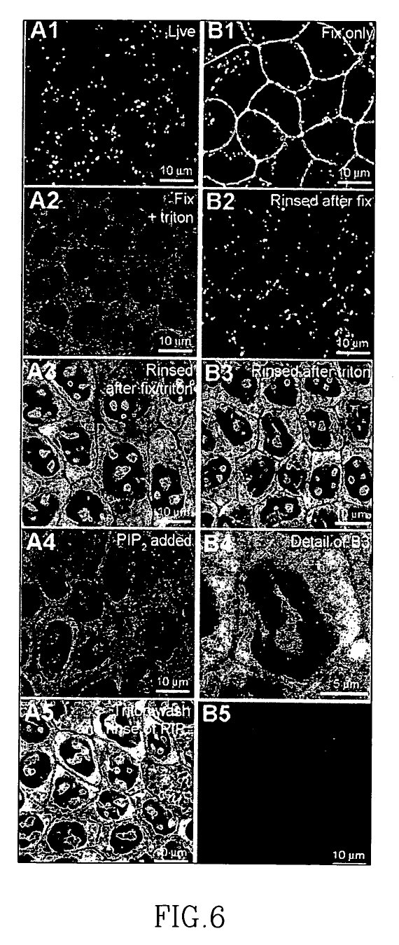

TRPV-1 agonists: Resineferitoxin (RTX) is a potent TRPV1 agonist that induces a transient inward current, that is desensitized in the presence of calcium. We tested the effect of RTX on GTTR penetration of cells to determine whether an agent that opens this channel to a cation current could enhance GTTR uptake. Cells were washed with calcium-free saline and treated with GTTR in the presence of several doses of RTX in calcium-free saline at pH 7.3. (No EGTA was present in the saline to bind residual calcium as the cells would have de-adhered from the coverglasses during treatment and washing. Thus there was a minor amount of calcium during treatment.) At 10⁻⁸ M RTX, GTTR uptake was significantly increased (FIG. 4, B2). At the higher dose of 10⁻⁷ M RTX, uptake was increased to a lesser extent (FIG. 4, B4), and at 10⁻⁶ M RTX, there was little or no change over control (FIG. 4, B4 and B1, respectively). The decrease in GTTR effect at higher doses might be explained by agonist desensitization due to the residual calcium present. In other experiment, calcium concentrations as low as 0.08 mM in the presence of RTX inhibited GTTR uptake. Anandamide (AND) is an endogenous cannabinoid and TRPV 1 agonist that produces a transient inward cation current and competes with both RTX and capsaicin for binding to the TRPV1 receptor. It was tested for its effect on GTTR uptake using the same protocol as for RTX. Consistent with its reported weaker binding to TRPV1, this ligand required higher doses to produce increases in GTTR uptake. At 10⁻⁶ M AND, and to a greater extent at 10⁻⁵ M AND, GTTR uptake was increased, although not to the level seen with RTX (FIG. 4, B5 and B6, respectively). At 10⁻⁴ M AND, GTTR uptake showed little, or no, increase over controls (FIG. 4, B7 and B1). These data show that TRPV1 channel agonists regulate gentamicin uptake in a manner similar to their reported stimulation of cation currents.

TRPV-1 antagonists: Several known TRPV1 antagonists were tested. Two specific antagonists, SB366791 and iodo-RTX, do not induce ion currents in tested cells. Both competitively reduce the binding of known TRPV1 agonists, and block the cation current induced by specific agonists. Both and SB366791 and iodo-RTX acted as agonists regarding GTTR uptake. At doses from 10⁻⁷ M to 10⁻⁵ M, SB366791 increased GTTR uptake significantly (FIG. 4, C2-C4). The effect of 1-RTX, which binds to TRPV1 with a higher affinity than SB366791, was dramatic (FIG. 4, C5-C7). At 10⁻⁵ M I-RTX the GTTR fluorescence was well over the upper limit of the available 0 to 255 gray scale when using parameters optimized for comparison of the other images in this figure. With both of these molecules, increased doses of these specific antagonists increased uptake (in contrast to the TRPV1 agonists). Neither showed “desensitization” in the presence of calcium. Ruthenium Red (RR) is a non-competitive TRPV1 antagonist that blocks numerous cation channels. Cells treated with 10 mM RR alone (FIG. 4, E1) took up less GTTR than controls (FIG. 4, D1). The same dose of RR also blocked GTTR increases stimulated by RTX, AND, SB366791, and I-RTX (FIG. 4, E2, E3, E4 and E5, respectively), although the AND effect was not completely blocked. Blocking of GTTR uptake by RR further demonstrated the involvement of cation channels in the penetration of GTTR into the cytoplasmic compartment of MDCK cells.

PIP₂

PIP₂ is well known to interact with gentamicin (S. Au et al., 1987, Biochim Biophys Acta. 902:80-6; M. Toner et al., 1988, Biochemistry, 27:7435-43; S. E. Williams et al., 1987, Hear Res. 30:11-8), probably, in part, via a cation/anion association. To test whether this association could influence GTTR uptake, cells were treated with 1 μg/ml GTTR in PBS into which 75 μg/ml of PIP₂ had been added prior to treatment. Control cells were treated without PIP₂. In this experiment, a dose of 1 μg/ml GTTR was used and cells were treated for 1 minute rather than 30 seconds. In addition, imaging parameters were set up to produce a bright image in the controls. The presence of PIP₂ during treatment caused a large reduction in GTTR uptake compared to the control (FIG. 5). This was not due to the trivial effect of fluorescence quenching, because treated cells were delipidated, as usual, by 0.5% Triton X-100 present in the fixative that should have removed the added PIP₂. Therefore, PIP₂ blocked GTTR uptake in the cells, and/or its binding at intracellular sites.

Gentamicin (average MW=469) and the conjugate GTTR (MW=1184) are much larger in size than the cations generally envisioned permeating TRP channels. However, a large body of evidence demonstrates that many factors besides size influence permeation of a particular species into a specific channel. These factors include hydration state/hydration energy (P. H. Barry et al., 1999, Clin Exp Pharmacol Physiol. 26:935-6; R. J. French and J. J. Shoukimas, 1985, J Gen Physiol. 85:669-98; X. Gong et al., 2002, J Physiol. 540:39-47; Z. Qu et al., 2000, J Gen Physiol. 116:825-44), electrostatic interactions of the permeant with side groups of amino acid residues within the pore (L. Guidoni et al., 1999, Biochemistry, 38:8599-604), and hydrogen bond exchanges between the permeant and amine side groups which have formed conformational hydrogen bonds with other side groups in the channel pore (D. B. Tikhonov et al., 1999, Biophys J. 77:1914-26). Furthermore, there are numerous reports of large organic cations (including fluorescent dyes) permeating various cation channels, with evidence that ionic size is only one of the factors predicting permeability (S. Balasubramanian et al., 1995, J Membr Biol. 146:177-91; J. E. Gale et al., 2001, J. Neurosci. 21:7013-25; C. J. Huang et al., 2000, J Gen Physiol. 115:435-54; B. S. Khakh et al., 1999, Nat Neurosci. 2:322-30; R. E. Marc, 1999, J Comp Neurol. 407:47-64; J. R. Meyers et al., 2003, J Neurosci. 23:4054-65; W. Qu et al., 2000, J Membr Biol. 178:137-50; C. Virginio et al., 1999, J Physiol. 519 Pt 2:335-46). Those studies suggest the clear likelihood that gentamicin, other aminoglycosides, and possibly other polyamine drugs can permeate cation channels. The data presented in this example demonstrate that cation channels, and in particular the TRPV1 vanilloid receptor channel, are involved in gentamicin penetration into cells.

Calcium

The calcium dilution series shown in FIG. 3, A1-A7 shows that extracellular calcium influences GTTR uptake into cells. A very low level of calcium is necessary for uptake, but even physiological levels (1.8 mM) were inhibitory, with higher levels almost completely blocking uptake. This could be due either (i) the two polycations competing for the same channel, (ii) the calcium regulating the open time of the relevant channels, or a combination of both. The data do, however, clearly implicate cation channels in the process of gentamicin penetrating into the cytoplasmic compartment of cells.

TRPV-1 Agonists

Protons, as well as specific agonists that bind to, and compete for, the TRPV1 binding site, produce cation currents though those channels. Similarly, we found that the specific agonists RTX and anandamide both stimulated GTTR uptake in calcium-free media, with a relative effectiveness consistent with their known affinities for the receptor binding site. In both cases, higher concentrations of agonist were less effective, suggesting agonist-induced closing or blockage of the channel. This is consistent with the known desensitization of agonist-induced currents, which occurs when agonists are tested in the presence of calcium. Although our experiments were done in nominally calcium-free buffer, we were unable to use a chelator such as EDTA, because EDTA caused cell islands to detach from the coverglasses and then not be available for observation. Thus, a small amount of calcium was certainly present in our nominally calcium-free buffer. In other experiments, as little as 0.08 mM added calcium in buffers blocked the stimulatory effect of these agonists on GTTR uptake.

TRPV-1 Antagonists

There are several TRPV-1 antagonists which compete with capsaicin or RTX for binding to the TRPV1 receptor. Iodo-RTX binds with high affinity. It induces no current in treated cells, and blocks RTX- or capsaicin-induced currents (P. Wahl et al., 2001, Mol Pharmacol. 59:9-15). SB366791 shows similar effects, but with a lower affinity for the binding site than I-RTX (J. B. Davis et al., 2001, Soc. Neurosci. Abstr. 27:910.5; C. J. Fowler et al., 2003, Biochem Pharmacol. 66:757-67). Surprisingly, both of these antagonists significantly increased GTTR uptake, with iodo-RTX effective at lower doses, consistent with their relative binding affinities. Unlike the agonists RTX and anandamide, no “desensitization” was observed at higher concentrations using these antagonists. Furthermore, in experiments not shown, the presence of calcium in the treatment buffer along with the antagonists did not block the stimulatory effect on GTTR uptake, unlike the agonists.

The effects of these specific antagonists could be a direct, as yet unexplained, effect within the receptor channel in which the gentamicin molecule itself alters the antagonist-receptor interaction, or the receptor conformation. Gentamicin is known to bind to PIP₂, which is a component of the TRPV-1 channel, and whose binding to the channel participates in blocking the channel (H. H. Chuang et al., 2001, Nature 411:957-62; E. D. Prescott et al., 2003, Science 300:1284-8). Gentamicin, in its interaction with the channel pore, may bind to and then remove PIP₂ from its pore binding site.

The non-competitive cation blocker Ruthenium Red reduces GTTR uptake in untreated samples and blocks the stimulatory effect of both agonists and antagonists, further supporting the conclusion that gentamicin enters cells via one or more cation channels. This also shows that the effect of the specific agonists and antagonists is directly on the channel, and not an indirect effect on some other molecular entity.

Extracellular PIP₂

The data reinforce the importance of PIP₂ in gentamicin's interaction with cells, and also demonstrate that the Texas Red-conjugated molecule is still able to form an ionic interaction with PIP₂.

Materials: The Texas Red-gentamicin conjugation procedure was optimized with regard to time, temperature, pH, and ligand/reactive fluorophore ratio to maximize labeling efficiency and to minimize the possibility of over-labeling the gentamicin. This insures maintenance of the polycationic nature of gentamicin, which is likely required for its biological activity. After conjugation, the reaction mixture was separated with the aid of C-18 reverse phase chromatography to isolate the conjugate and thereby eliminate competition from unlabeled gentamicin, or potential contamination by unreacted Texas Red. The isolated GTTR is then aliquoted, dried, and stored dessicated, dark and at −20° C. All of this provides a reliable reagent for testing gentamicin distribution.

Methods: Cells were seeded into 8-well coverglass chambers at 3000 cells/well and grown for 5 days. Cells were subconfluent, but had time to develop tight junctions and become columnar.

### EXAMPLE 2

### In Vitro Analysis of Uptake of Gentamicin by Immortal Kidney Cell Line

Two cell types were used in these studies, an opossum kidney proximal tubule (OK) clone and a canine kidney distal tubule (MDCK) clone. The OK proximal tubule cell line was chosen because of the known clinical toxicity of aminoglycosides in the kidney proximal tubules (Fabrizii et al., 1997, Wien Klin Wochenschr. 109:830-5; Morin et al., 1984, Chemioterapia. 3:33-40), and the retention by OK cells of the PTH responsiveness characteristic of the kidney proximal tubule (Paraiso et al., 1995 B.B.A. 1266:143-147; Silverstein et al., 2000 Horm. Res. 54:38-43). The distal tubule cell line was used because, although far less subject to AG-induced cell death, the distal tubule is subject to numerous acute effects (H. S. Kang et al., 2000, Can J Physiol Pharmacol. 78:595-602; Kidwell et al., 1994, Eur J Pharmacol. 270:97-103; Quamme, 1986, Magnesium 5:248-72). Both were cloned from cultures that had been maintained for extended periods in the absence of the aminoglycoside streptomycin, a common bacterial prophylactic component of many culture media. This was done to optimize the response of cells to the AG gentamicin. Although no morphological change was observed in MDCK cells cultured without streptomycin, the OK cells became morphologically much more epitheloid after extended streptomycin-free culture.

Cytoplasmic and Intranuclear Binding Compared to Vesicular Uptake of GTTR Vesicular

MDCK cells were treated with 1 μg/ml purified GTTR for 2.5 hours in complete medium at 37° C., then washed 3× with complete medium. Similar to previous studies using Texas Red-labeled gentamicin (Sandoval et al., 1998, J. Am. Soc. Nephrol. 9:167-174; Sandoval et al., 2002, Am. J. Physiol. Renal. Physiol. 279:F884-90; Sandoval et al., 2000, Am. J. Physiol. Renal Physiol. 283:F1422-9), we found GTTR in a punctate, endosome-like distribution in live cells (FIG. 6, A1). Cells fixed with 4% formaldehyde (FA) alone and imaged either before (FIG. 6, B1) or after (FIG. 6, B2) washing in buffer exhibited the same vesicular labeling pattern as reported previously (Sandoval et al., 1998, J. Am. Soc. Nephrol. 9:167-174; Sandoval et al., 2002, Am. J. Physiol. Renal. Physiol. 279:F884-90; Sandoval et al., 2000, Am. J. Physiol. Renal Physiol. 283:F1422-9). Interestingly, in the cells remaining in fixation buffer (FIG. 6, B1), labeling was also observed at the cell periphery where it was seen in neither the live cells, nor the fixed and washed cells.

Cytoplasmic and Intra-Nuclear

In contrast, cells fixed with formaldehyde containing 0.5% Triton X-100 (FATX) revealed an entirely novel distribution. With this fixation and delipidation protocol, no intracellular vesicles were observed. Instead, GTTR was observed within the cytoplasm and on distinct intra-nuclear structures (FIG. 10, A3). In these images, the cytoplasmic labeling reveals little substructure, but the intra-nuclear binding pattern was complex, showing round or ovoid structures with trans-nuclear tubuloid structures appearing to connect to them (FIG. 6, A3, A5, B3, B4). Cells examined while still in FATX exhibited the same distribution, but the fluorescence was greatly quenched (FIG. 6. A2). Cells that had been fixed in FA alone and washed (FIG. 6, B2) were then exposed to Triton alone, and washed with buffer. In these cells, the vesicular fluorescence had disappeared and the cytoplasmic/nuclear staining was visible (FIG. 6, B3). As before, fluorescence was quenched when the cells remained in Triton. This demonstrates that cytoplasmic/nuclear GTTR was present, but not visible, in the FA only images, and also that some characteristic of Triton quenches this fluorescence. It also demonstrates that penetration of GTTR into the cytoplasm was not an artifact of Triton being present during the fixation process. Cells treated with hydrolyzed TR alone had negligible fluorescence when imaged live (FIG. 6, B5), or after formaldehyde fixation or after FATX treatment (FIG. 6, B6).

Quenching of GTTR Fluorescence by Ionic Lipids

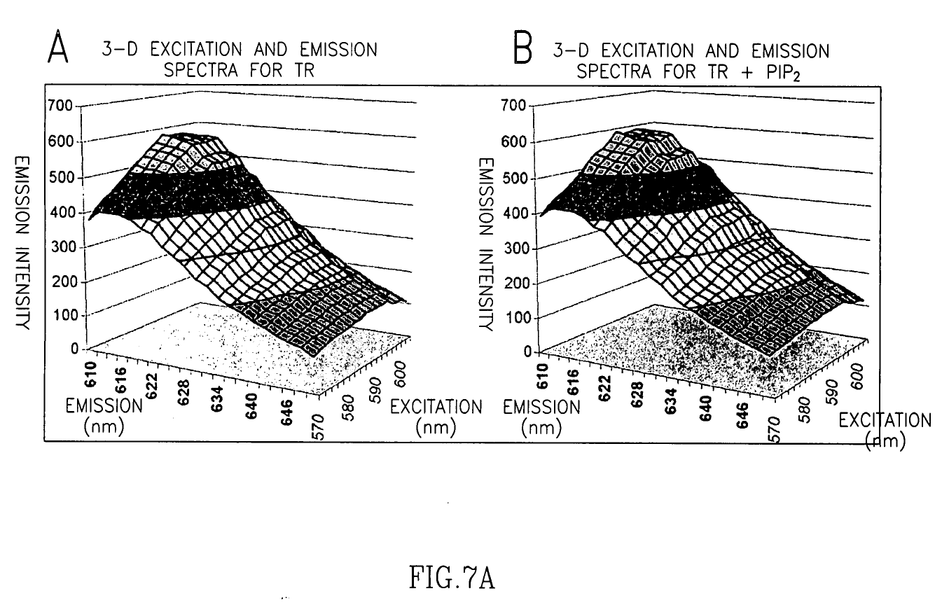

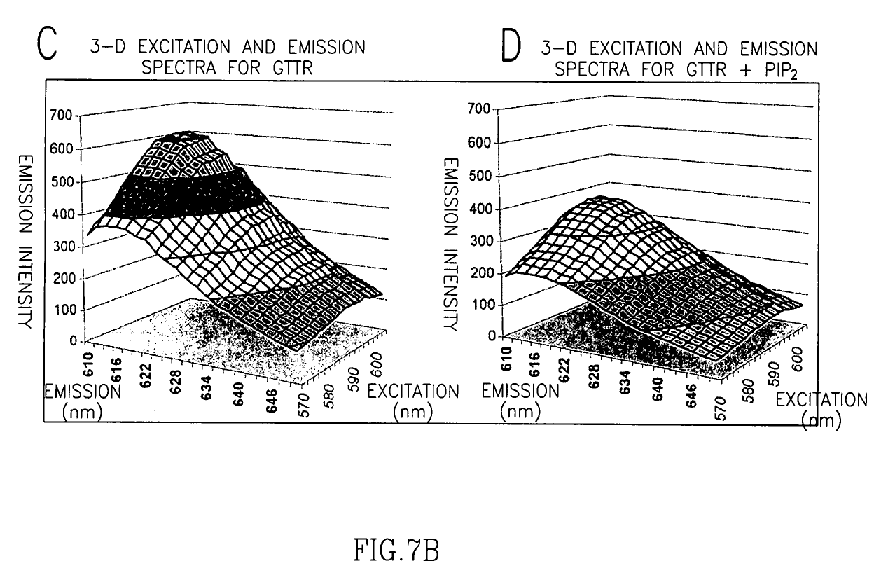

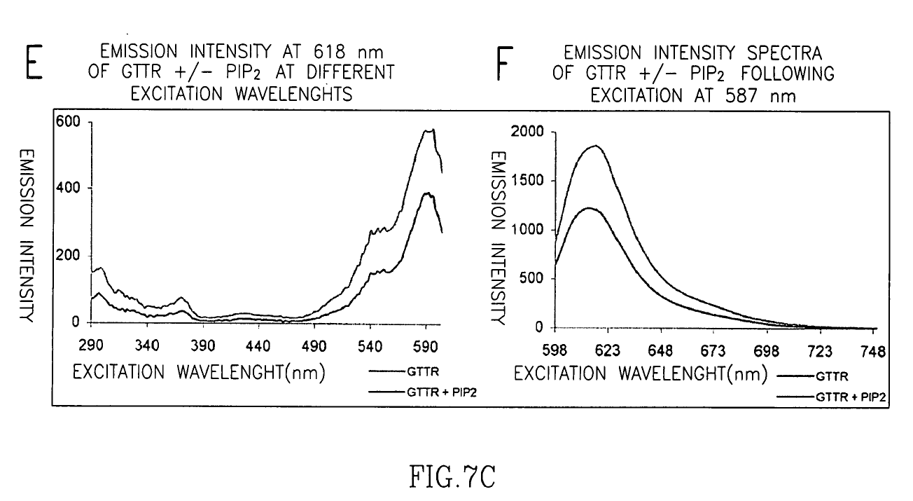

Because Triton X-100, a detergent that removes cellular lipids, is an anionic lipid, we reasoned that anionic cellular lipids might be quenching GTTR fluorescence in the live and PFM-alone fixed specimens, as suggested by the Triton X-100 quenching of fluorescence in FIG. 6, A2. There are reports of structural and functional associations of the polycationic gentamicin with cellular anionic phospholipids, and in particular with phosphatidylinositol-4,5-bisphosphate (PIP₂). PIP₂ is a functionally required part of numerous ion channels (D. W. Hilgemann et al., 2001, Sci STKE 2001:RE19), and is found associated with cytoskeletal elements and within nuclei (E. Caramelli et al., 1996, Eur J Cell Biol. 71:154-64; G. Mazzotti et al., 1995, J Histochem Cytochem. 43:181-91; W. Xian et al., 2002, J Mol Biol. 322:755-71; H. L. Yin et al., 2003, Annu Rev Physiol. 65:761-89). To determine whether such an association might explain the lack of cytoplasmic/nuclear fluorescence in living or FA-only fixed cells, we attempted to quench GTTR fluorescence with PIP₂. Cells which had been fixed with FATX then rinsed (as in FIG. 6, A3) were treated with 1 mg/ml PIP₂ for 1.5 hour and imaged while still in the PIP₂ solution (FIG. 6, A4). PIP₂ clearly quenched the GTTR fluorescence. This was not due to removal of the GTTR, because when the PIP₂-treated cells were again delipidated with 0.5% Triton X-100 and washed, GTTR fluorescence regained its former brightness (FIG. 6, A5). Reduction of GTTR fluorescence by PIP₂ was due to fluorescence quenching and not by excitation or emission spectra shifts. This was ascertained using 3-d scanning spectroscopy with a excitation range of 570-604 nm (bandwidth 5 nm) and an emission bandwidth of 5 nm between of 610-650 nm. PIP₂ in solution with GTTR exerted a quenching effect at all wavelengths (FIG. 7D, E, F). This was probably due to binding of the amphipathic polyanion PIP₂ to the amphipathic polycation gentamicin, since no such quenching effect was observed when PIP₂ was combined with TR alone in solution (FIG. 7B).

Characteristics of Cytoplasmic and Nuclear Binding: Saturability, and Time and Temperature Effects

Saturability in the binding of a ligand demonstrates the existence of a limited number of binding sites and is the hallmark of specificity. Saturability is demonstrated if binding of a labeled version of the ligand can be serially reduced as a function of increasing quantities of the native, unlabeled ligand. Such data also demonstrate that the labeled ligand remains sufficiently bio-relevant that its distribution is a valid report of the distribution of the unlabeled molecule. Time and temperature modulate biological processes, for example, the event(s) involved in crossing a barrier such as the cell plasma membrane. In particular, at a low temperature (cells held over ice) endosomal traffic would be halted, but permeation through pores or channels could continue, albeit more slowly.

Binding Specificity

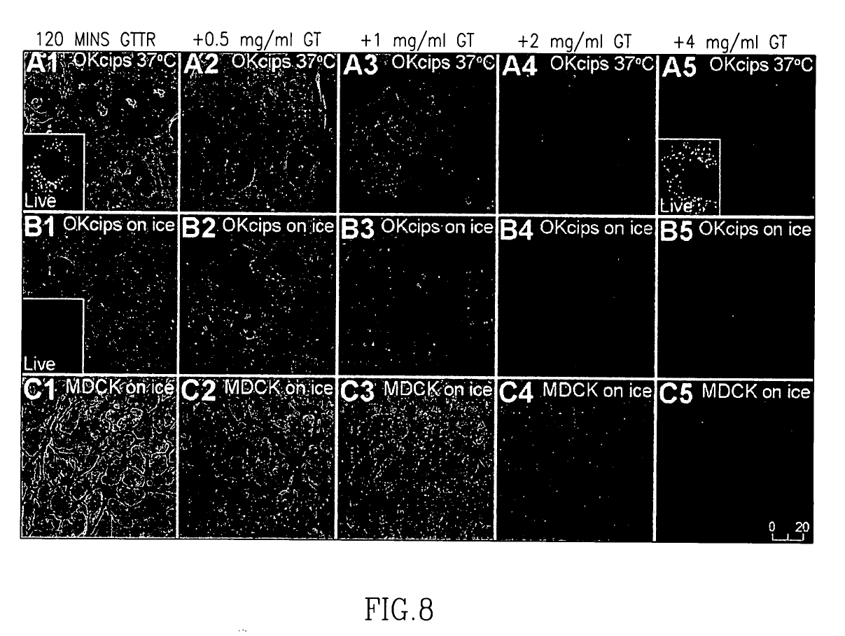

Both OK and MDCK cells were used in these studies. Both cell lines were treated with GTTR at 1 μg/ml in complete culture medium for two hours at either 37° C. or over ice. These cells were also treated with a dose range of 0 to 4000 μg/ml of unlabeled GT. Cells were washed and imaged live, then fixed with FATX, and washed again with buffer prior to re-imaging (as in FIG. 6, A3). Only at 37° C., was there endosomal accumulation of GTTR (FIG. 8, A1 insert) and this accumulation was not visibly altered by even the highest doses of unlabeled GT (4 mg/ml; FIG. 8, A5 insert). After FATX fixation and wash, cytoplasmic and nuclear fluorescence was observed in both cell types and at both temperatures (FIG. 8, A1, B1, and C1). Fluorescence was reduced in cells treated on ice, but, notably, still occurred. At both temperatures and in both cell lines, increasing doses of unlabeled GT serially reduced the amount of GTTR observed in both the cytoplasmic compartment and within nuclear structures (FIG. 8, A2-5, B2-5, and C2-5). Thus, cytoplasmic, but not endosomal, uptake of GTTR was saturable. Cells treated while on ice then imaged live exhibited no endosomes (FIG. 8, B1 insert), as expected, showing that cytoplasmic uptake occurs in the absence of the formation of endosomes.

Time and Temperature

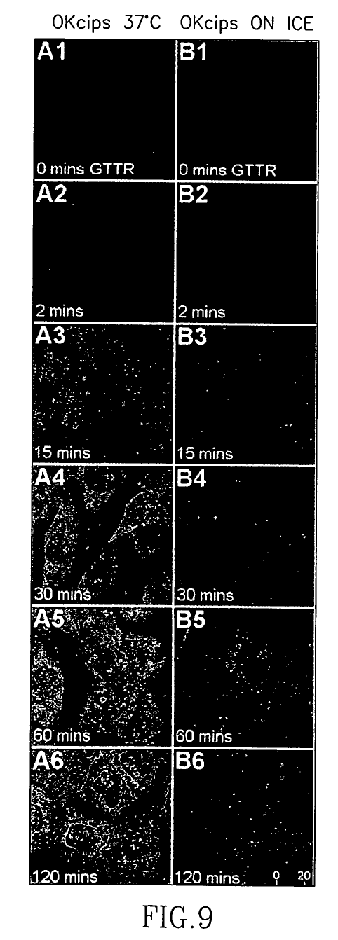

Binding of GTTR increased over time both at 37° C. (FIG. 9, A1-6) and (more slowly) on ice (FIG. 9, B1-6). At 37° C., cytoplasmic binding occurred prior to visible uptake into endosomes.

Cytoplasmic Penetration

This example describes the distribution of fluorescently labeled gentamicin at intracellular sites not previously described. GTTR was observed within the cytoplasm and at intra-nuclear sites. The distribution was not observed in live cells, but only after both fixation and detergent delipidation. Finding gentamicin in the cytoplasm is not inconsistent with earlier studies, using radiolabeling or biochemical extraction, (G. Decorti et al., 1999, Life Sci. 65:1115-24; D. N. Gilbert et al., 1989, J Infect Dis. 159:945-53; R. P. Wedeen et al., 1983, Lab Invest. 48:212-23) (Steyger et al 2003 J.A.R.O., in press) or in the nucleus (Steyger et al 2003 J.A.R.O., in press); D. Ding et al., 1995, Zhonghua Er Bi Yan Hou Ke Za Zhi 30:323-5; D. Ding et al., 1997, Zhonghua Er Bi Yan Hou Ke Za Zhi 32:348-9). Penetration of gentamicin into the nucleus is supported by clinical studies in which gentamicin was able to by-pass premature stop codons in genetic diseases (J. P. Clancy et al., 2001, Am J Respir Crit Care Med 163:1683-92; P. R. Clemens et al., 2001, Curr Neurol Neurosci Rep. 1:89-96; K. M. Keeling et al., 2002, J Mol Med. 80:367-76; A. Schulz et al., 2002, J Clin Endocrinol Metab. 87:5247-57). However, penetration of gentamicin directly into the cytoplasm of living cells is contrary to recent reports showing gentamicin-Texas Red uptake by kidney cells only via endosomes, and without any cytoplasmic uptake (Sandoval et al., 1998, J. Am. Soc. Nephrol. 9:167-174; Sandoval et al., 2002, Am. J. Physiol. Renal. Physiol. 279:F884-90; Sandoval et al., 2000, Am. J. Physiol. Renal Physiol. 283:F1422-9). Indeed, in our experiments, cells or fixed with FA alone, negligible cytoplasmic fluorescence was observed (FIG. 6, B1 and B2). Thus, detergent delipidation during, or following, fixation was required to reveal this novel cytoplasmic and nuclear distribution pattern.

An important difference between the present invention and the earlier reports using fluorescently labeled gentamicin is the “permeabilization” used (Sandoval et al., 1998; Sandoval et al., 2002; Sandoval et al., 2000). In that study, Triton X-100 was used at a concentration of 0.05% for 10 minutes. In this example, a 0.5% concentration was used for at least 30 minutes, and then thoroughly rinsed, thus more effectively removing cellular lipids.

Fluorescence Quenching

Triton X-100 and PIP₂ reduced the fluorescence of GTTR. The Texas Red molecule is known to exhibit little change in fluorescence emission in response to environmental conditions, such as changes in pH (Haugland et al., 1996), although its fluorescence can be diminished somewhat if it becomes so concentrated it is self-quenching. We also find no reports of environmental sensitivity when Texas Red is conjugated to large molecules, such as antibodies. But, gentamicin, a mixture of 3 isoforms with an average MW of 469, is a polyamine, with 2 or 3 amine side groups remaining after conjugation with Texas Red. Deprotonation of these amines could alter the fluorescent efficiency of a fluorophore covalently attached to the gentamicin. In other experiments, comparing fluorescence of GTTR and unconjugated Texas Red in solution, with the aid of fluorimetry, we found GTTR to be far more pH sensitive that Texas Red.

In both confocal imaging and fluorimetry, however, both excitation and emission wavelengths are selected with band-pass filters. This method of excitation/detection does not allow us to distinguish between (apparent) fluorescence quenching and an environmentally-induced spectral shift in the excitation or emission spectrum, or both. Such shifts could produce peaks that would miss the band pass filters and appear as quenching even if emission were enhanced at a different wavelength. However, spectral scans of GTTR in solution with or without PIP₂ over an excitation range of 570-604 nm and emission range of 610-650 nm produced 3-dimensional fluorescence maps which showed clearly that PIP₂ attenuated GTTR fluorescence at all wavelengths (FIG. 7). PIP₂ had no effect on Texas Red alone in solution, indicating that PIP₂ was interacting with the gentamicin moiety of GTTR. Yet, in those experiments, and in the image in FIG. 6, A3, PIP₂ did not completely block GTTR fluorescence, although almost no GTTR fluorescence was observed in live cells treated at temperatures incompatible with endosomal uptake. In solution, much higher concentrations of PIP₂ might have completely blocked fluorescence. In vivo, other lipids (e.g., phosphatidylserines) or cellular quenching mechanisms may be involved. Additionally, PIP₂ may not have bound as effectively to GTTR which had been cross-linked, via one or more of its amine groups, with its intracellular binding sites (in fixed cells), as it does with free GTTR, which retains 2 or 3 amine groups (prior to fixation, while cells are alive).

Saturability of the Cytoplasmic Compartment

With either the proximal, OK, or distal, MDCK, kidney cell lines, binding of GTTR was saturable at both cytoplasmic and intra-nuclear sites. GTTR binding serially decreased as a function of increasing concentrations of unlabeled gentamicin in the culture media. Competition with the native molecule shows that intracellular gentamicin binding sites are limited in number, and that the labeled molecule retains the biological characteristics of the native molecule, at least with regard to uptake and distribution (GTTR is a tracer, and would not be used to study toxicity or other activities). This demonstrated the biological specificity of GTTR binding at these sites.

At 37° C., we also observed vesicular uptake of GTTR over time. Unlike one previous report (Sandoval et al., 1998), we were unable, in numerous experiments, to see any significant reduction in vesicular uptake, even using high excesses of native gentamicin (>4000×). One explanation for this difference may be that extended gentamicin treatment (about 12 mg/ml excess) of LLC-PK1 cells, as used by Sandoval et al. (1998), inhibits endocytosis, and could have blocked endocytotic uptake of labeled gentamicin in a non-competitive manner (S. A. Kempson et al., 1989, J Biol Chem. 264:18451-6). At 4 mg/ml gentamicin, we observed flattening of cells, which slightly altered the apparent distribution of vesicles, so a small decrease in vesicle number would be difficult to observe or document. But, overall, unlike the near extinction of cytoplasmic and nuclear binding of GTTR at the highest doses of unlabeled gentamicin, vesicular uptake of GTTR was little changed. This suggests that a large fraction of the vesicular uptake is associated with fluid-phase endocytotic uptake, since it is not saturable, confirming a previous report (G. Decorti et al., 1999, Life Sci. 65:1115-24). This non-specific endocytotic uptake of AGs is also consistent with the observation that GTTR in the vesicular compartment is not cross-linked to any cellular component during aldehyde fixation and ultimately washed away. Over time in buffer (days), vesicular staining declined and gradually disappeared. This indicates that the conjugate seen under those conditions was not sufficiently closely bound to cellular structures to permit formaldehyde cross-linking.

Temperature and Time

Unlike any previous study, we report uptake of gentamicin into cells held on ice. An earlier study (Decorti et al., 1999), compared binding of gentamicin to cells at 37° C. and at 4° C. In that study, gentamicin in cells scraped from Petri dishes after treatment was quantified using immunoenzymatic techniques. Their interpretation was that GT readings from cells incubated at 4° C. represented plasmalemmal binding. No microscopy was done to determine spatial distribution, but the data could alternatively support penetration of gentamicin at the cold temperature. In the present example, at both 37° C. and on ice, GTTR could be seen in both the cytoplasm and within nuclei. Uptake increased as a function of time over hours, more slowly within cells held over ice, as would be expected of a biological process. The finding of gentamicin uptake at cold temperatures further suggests that endosomes were not involved in the process. Indeed, LLC-PK1 cells deficient in megalin, a receptor that mediates endocytotic uptake of AG (Girton et al., 2002; Moestrup et al., 1995), or various inhibitors of clathrin-dependent endocytosis (monensin, phenylarsine oxide, dansylcadaverine) and caveolae-mediated endocytosis (nystatin), failed to prevent gentamicin uptake (Decorti et al., 1999; Girton et al., 2002). This leads us to consider cation channels (especially polycation channels) as the means for gentamicin entry into the cells.

The present example demonstrates that gentamicin enters cells via an endosome-independent pathway and binds to sites within the cytoplasm and nucleus.

Cell Culture Reagents

Cells were cultured in Dulbecco's Minimal Essential Medium μ (MEMμ), purchased from Invitrogen, Carlsbad, Calif. Fetal bovine Serum (F2442), ITS (I-3146), Gentamicin Sulfate (G1264) were purchased from Sigma Alderich, St. Louis, Mo., Texas Red succinimidal ester was purchased from Molecular Probes, Eugene, Oreg.

Cell Culture

Canine kidney distal tubule MDCK cells are commercially available. Opposum proximal tubule derived kidney cells (OK) were purchased from American Type Culture Collection. OK and MDCK were cultured antibiotic free or in ciprofloxin (cip) supplemented medium. The cell lines were cultured in Dulbeccos Minimal Essential Medium (MEMμ, 11095-080 Invitrogen, Carlsbad, Calif.) with 10% FBS and kept at 37° C. with 5% CO2, 95% air. The OK media was supplemented with ITS and interferon. Plates used for OK cells were coated with 0.2% gelatin by incubating in gelatin for 2 or more hours at 37° C. Plates used for the Okcips were also coated with a 10% collagen, 10% FBS, 80% NaCl₂ solution by adding solution to the plates for one minute, aspirating, and drying under the laminar flow hood for two hours.

### EXAMPLE 4

### In Vivo Protection from Ototoxicity Through Block of TRP Channels

Two groups of mice are administered a cocktail of gentamicin plus TRP channel blockers or gentamicin alone. Cochleae from mice given the TRP channel blockers harbor significantly greater numbers of surviving hair cells and show lower incidence of gentamicin-induced apoptosis or necrosis than those given gentamicin alone. Such data would demonstrate that TRP channel blockers can attenuate the ototoxic actions of gentamicin in the auditory system of the mouse, producing a preventative treatment for chemical-induced hearing disorders.

### EXAMPLE 5

### In Vivo Protection of Cochlear Hair Cells from Ototoxic Effects of Gentamicin Through Block of TRP Channels in Guinea Pig

Pigmented guinea pigs (250-400 g) without evidence of middle ear infection are used in this study. For the gentamicin study, animals are implanted with osmotic pumps (Alzet, Palo Alto, Calif., model 2ML2, 5 μl/h) filled with artificial perilymph (n=5) and gentamicin (300 mM) for 24 h followed by perilymph infusion for 2 weeks (n=15). A group of animals is injected with TRP channel blockers before implanting the pump. Other animals are implanted with osmotic pumps filled with gentamicin (300 mM) whereas a third set of animals is implanted with pumps filled with artificial perilymph. After 6 h the animals are anaesthetized with pentobarbital (30 mg/kg), then perfused intracardially with body temperature physiological saline followed by a solution of 5% glutaraldehyde and 4% paraformaldehyde in 0.1 M phosphate buffer with 4 mM MgCl₂. The cochleae are removed and postfixed with 2% osmium tetroxide in 0.1 M phosphate buffer, embedded in Agar 100 Resin. Sections are stained with toluidine blue and analysis of the afferent dendrite morphology is made. Analysis is performed with a Zeiss Axioscope microscope equipped with oil immersion objectives.

Auditory brainstem response (ABR) thresholds are measured before implanting the osmotic pump, 24 h after as well as 1 and 2 weeks after implanting the pump. After the final auditory brainstem measurement the cochleae are removed for morphological analysis. A Student's t test is performed to determine the statistical significance of the data. Differences are considered statistically significant when the P value is 0.05 or below.

Implantation and Filling of the Osmotic Pump. The microosmotic pump (Alzet, model 2ML2; 5 μl/h) is used according to standard methods. Briefly, guinea pigs are anaesthetized with rompun (10 mg/kg, i.m.), and ketamine (50 mg/kg, i.m.) and 10% xylocaine containing adrenaline are applied locally. The right bulla is exposed postauricularly and a 2-mm hole is drilled through the bone of the bulla and a small hole (≈0.2 mm) is made to access the scala tympani in the basal turn. A steel needle (0.2 mm outer diameter) is connected to a plastic tube and is inserted into the hole and fixed with dental cement (Fuji I, Tokyo). A s.c. pocket is made to accommodate the pump in the back region and the catheter between the bulla and the microosmotic pump is fixed by superglue. The composition of the artificial perilymph is as follows: 137 mM NaCl, 5 mM KCl, 2 mM CaCl₂, 1 mM MgCl₂, 12 mM NaHCO₃, 11 mM glucose; the pH is adjusted if necessary to 7.4.

ABR. After an i.m. injection of 50 mg ketamine and 10 mg rompun per kg body weight ABR responses are recorded s.c. with stainless-steel electrodes as the potential difference between an electrode on the vertex and an electrode on the mastoid, while the lower back serves as ground. The body temperature of the animals is maintained at 38° C. by using an isothermic heating pad. Stimulus intensity is calibrated with a one-quarter-inch condenser microphone (Bruel & Kjaer Instruments, Marlborough, Mass., model 4135) and are expressed in peak SPL re: 20 μPa. The stimulus signal is generated through Tucker-Davis Technologies (Gainesville, Fla.) equipment controlled by computer and delivered by an earphone (Telephonics TDH 39, Farmingdale, N.Y.). The stimuli are delivered through a closed acoustic system sealed into the external auditory meatus. The evoked response is amplified 100,000 times and averaged 2,048 sweeps in real time at a digital signal process (DSP32C) with a time-domain artificial rejection. Stimuli are presented at an intensity well above threshold and then decreased in 10-dB steps until the threshold is approached and then in 5-dB steps until the ABR wave disappears. Threshold is defined as the lowest intensity at which a visible ABR wave is seen in two averaged runs.

Morphological Analysis of the Cochlea. After the final auditory brainstem measurement, cochleae from the oto-protected group and from the control group are removed after cardiac perfusion with 4% paraformaldehyde and postfixed for 2 h in 4% paraformaldehyde. The cochleae are decalcified, embedded in paraffin, sectioned at 4 μm, and stained with toluidine blue. Dendrites under inner hair cells are visualized with an oil immersion microscope (Zeiss Axioscope under ×100 objectives).

To quantify the hair cell loss after gentamicin damage between the oto-protected and the control group, cochleae are placed in 4% paraformaldehyde in PBS (pH 7.4) for 1-2 h. The cochleae are rinsed in PBS and the bone is dissected away. The tissue is then exposed to 0.3% Triton X-100 for 10 min, rinsed, and incubated in fluorescently labeled phalloidin (tetramethylrhodamine B thiocyanate, TRITC) (1:100) (Molecular Probes) for 30 min and rinsed several times. The organ of Corti is dissected into ½-¾ coils and placed on a microscope slide in Citi-flour, and covered with a coverslip and sealed. All hair cells throughout the cochlea are examined by using a ×40 objective, and the percent hair cell loss per mm distance from the round window is then plotted on a cochleogram. An estimated frequency map also is indicated where the 9-mm distance from the round window represents the 8-kHz region, the 11-mm region represents the 4-kHz region, and the 13-mm region represents the 2-kHz region.

Although the inventors are not bound by a particular mechanism, based on the disclosure herein one therapeutic strategy is based on the concept that multiple TRP channels participate in gentamicin update. Thus, cocktails of blockers would be preferred to effectively prevent oto- and nephrotoxicity. These blockers in some embodiments will be mixtures of TRP-specific blocking agents, non-specific agents such as divalent cations (Ca, Mg, Zn), or peptide constructs.

For aminoglycoside antibiotics, cocktails are designed in accordance with specific TRPs in inner ear and kidney, based on the rationale that mammalian cell update it different than bacterial uptake. The objective is complete prevention of toxicity. For oto- and nephrotoxic anti-neoplastics, cocktails are designed in accordance with either the specific tumor type or the specific patient, based on the use of a diagnostic kit. Under this rationale, both normal and tumor cells are mammalian, so they will not use TRPs abundant in a specific tumor. The objective is to improve the therapeutic index.

### EXAMPLE 6

### Uptake of Gentamicin by Bullfrog Secular Hair Cells In Vitro

Gentamicin sulfate (Sigma; 50 mg/ml in K₂CO₃, pH 9) and succinimidyl esters of Texas Red (Molecular Probes; 2 mg/ml in dimethyl formamide) were agitated together overnight to produce a gentamicin-Texas red conjugate (GTTR). Typically, 4.4 ml of 50 mg/ml (final volume) gentamicin sulfate (GT) was mixed with 0.6 ml of 2 mg/ml Texas Red esters (TR) to produce an approximately 300:1 molar ratio of GT:GTTR. A high ratio of free gentamicin to TR esters ensures a minimum of unbound TR molecules, and a binding ratio of 1 TR molecule to 1 GT molecule (Sandoval et al. 1998). Gentamicin sulfate has three isoforms with molecular weights of (C1) 449.5, (C2) 463.5, and (C1a) 477.6. Texas Red succinimidyl esters have a molecular weight of 817. When combined, the conjugates have (rounded) molecular weights of 1165, 1179, 1193, respectively, after loss of the carbonyl amine from the reactive TR. Gentamicin has 3 or 4 amine groups depending on the isoform, and the conjugation of a TR molecule to a gentamicin amine group reduces the ionic charge of the conjugated molecule by one for each amine group conjugated to TR (generally one) and proportionately increasing its hydrophobicity. After conjugation, the GTTR conjugate is typically still a polyamine and a polycation.

Saccule Explantation

Bullfrogs (Rana catesbeiana) were anesthetized with 0.2% MS-222 and chilled before decapitation. The temporal bones were excised in cold oxygenated HEPES-buffered saline (HBS) containing 110 mM Na+; 2 mM K+; 4 mM Ca2+; 120 mM Cl−; 3 mM D-glucose, and 5 mM HEPES (pH=7.25; 220 mOsmols). Each saccule was isolated and the otolithic membrane removed by proteolytic digestion for 20 minutes in oxygenated 50 μg/ml subtilopeptidase BPN (Sigma) at room temperature. Saccular explants were then incubated in Wolf-Quimby culture medium (containing 1 mM Ca2+; Life Technologies) supplemented with 100 μg/ml ciprofloxacin in a 5% CO₂ environment at 25° C. (Steyger et al. 1997; Steyger et al. 1998). Gentamicin-treated explants were incubated in the above culture media, supplemented with 300 μg/ml GT/GTTR.

Confocal Microscopy

For confocal microscopy, excised saccular explants were individually pre-loaded (for 40 minutes) with 50 μM MitoTracker, 50 nM Lysotracker Green, 1 μM NBD-ceramide and 1 μM ERtracker (Molecular Probes) in Wolfe-Quimby amphibian culture media to label lysosomes, Golgi bodies and endoplasmic reticulum, respectively, prior to incubation with 300 μg/ml GT/GTTR for 2 hours. Explants were either (i) fixed in 4% formaldehyde (MitoTracker Green- and Lysotracker Green-loaded explants) and mounted; or (ii) placed in chambered coverslips and directly observed live (NBD-ceramide- and ERtracker-loaded explants), using a Bio-Rad MRC 1024 ES laser scanning confocal system attached to a Nikon Eclipse TE300 inverted microscope. For immunocytochemistry of gentamicin, explants were incubated with 300 μg/ml unconjugated gentamicin for 30 minutes or 2 hours, fixed, ice-cold acetone-permeabilized, and processed for indirect fluorescence immunocytochemistry. Explants were immunoblocked with 10% horse serum and 1% bovine serum albumin (BSA) in 0.02 M PBS for 30 minutes, and incubated with anti-gentamicin antibodies (American Qualex, CA) overnight. After washing in 1% BSA-PBS, explants were incubated in Alexa-568-conjugated goat anti-rabbit secondary antibodies. Subsequently, organs were labeled with Alexa-660-conjugated phalloidin and/or Sytox Green (Molecular Probes, OR), prior to mounting in VectaShield (Vector Laboratories) and confocal imaging. Immunocytochemical controls included: (i) primary antibody labeling of saccular explants incubated with normal culture media only, and (ii) gentamicin-adsorbed primary antibody labeling of GT-treated explants. Confocal images of double-labeled specimens were collected sequentially to prevent bleed-through and cross-talk between the different fluorescent probes, using a ×60 [numerical aperture (N.A.) 1.4] objective lens in 1024×1024 pixel frames with an xy resolution=240 nm and xz resolution=400 nm, and post-processed using the Bio-Rad LaserSharp imaging software. Co-localization analysis was performed only on individual optical sections within a focal series. Pixels containing both red (e.g., GTTR or immunolabeled GT) and blue (phalloidin-Alexa-660) or green (Sytox Green) intensities above a user-defined threshold appeared as white within a colorized merged image for each optical section, indicating which pixels were sites of co-localization of the two chosen fluorophores.

Resolution of the Confocal Microscopy System

To determine the observed xy resolution for the Bio-Rad MRC 1024 confocal system attached to a Nikon TE300 microscope, sub-resolution fluorescent beads (0.175 μm; Molecular Probes) mounted under 1.5 coverslips were imaged, and the Full Width Half Maximum (FWHM) of several fluorescent specks were obtained for each lens used. The observed z-axis resolution was obtained by blue reflection imaging of an ultra-thin sputter-coated cover-slip (<30 nm), and subsequently obtaining the FWHM (Pawley 1995). The FWHM is derived from a line intensity graph of the sub-resolution target, where the observed optical resolution equals the width between the two slopes, approximately halfway between baseline and peak fluorescence of a sub-resolution fluorescent bead. The y-coordinate is derived using the following equation:
(Fₘₐₓ−F_{bkg})/2+F_{bkg}). [1]

Therefore, the optical resolution in μm is:

x-coordinate of Slope 2−x-coordinate of Slope 1 (Pawley 1995).

Immuno-Electron Microscopy

Saccular explants were incubated with 300 μg/ml unconjugated GT for 2 hours prior to washing and fixation in 4% paraformaldehyde plus 0.5% glutaraldehyde in 0.1 M phosphate buffer for 2 hours. Samples were dehydrated through an ascending alcohol series at progressively lower temperatures, culminating at −40° C. Subsequently, samples were infiltrated with LR Gold over 72 hours, and polymerized with UV-light for 48 hours, using the Leica AFS low-temperature embedding system. Ultra-thin sections were obtained on an ultra-microtome, collected on nickel grids, passaged several times through distilled water, and subsequently several times with 5 mM Tris (TBS). Grids were then immunoblocked with 20% normal goat serum in TBS for 30 min, and incubated overnight with gentamicin antibodies in 1% BSA/TBS at 4° C. Grids were rinsed three times in TBS and incubated in gold-labeled secondary antibodies (15 nm gold particles conjugated to goat anti-rabbit IgG, diluted 1:100; Ted Pella) in 1% BSA in TBS for 1 hr. Immunocytochemical controls included (i) replacing primary antibodies with gentamicin-adsorbed primary antibodies, or (ii) primary antibody labeling of sections cut from embedded explants incubated in normal culture media only. Grids were washed in TBS and water, stained with 2% aqueous uranyl acetate, and observed in a Philips CM 100 transmission electron microscope.

Specificity of GTTR Labeling

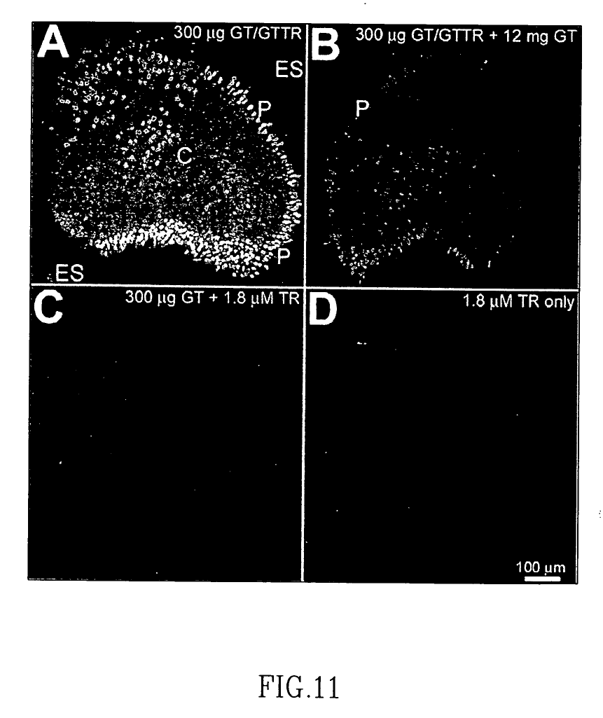

Control explants were imaged using the same confocal settings for laser power, iris size, gain, and black levels as the contra-lateral saccular explant contemporaneously treated with GTTR. When saccular explants were incubated with 300 μg/ml GT/GTTR (300:1 molar ratio) for 30 minutes prior to fixation and mounting, an intense band of fluorescence was present around the edge of the sensory epithelium (FIG. 11A), with less intense fluorescence within the central region of the saccule (FIG. 11A). There was little fluorescence in the extra-sensory epithelium (FIG. 11A).

Explants incubated with 300 μg/ml GTTR plus a 40-fold excess of unconjugated GT (i.e., 12 mg/ml) displayed reduced fluorescence in the sensory epithelium, particularly in the peripheral regions (FIG. 11B). Explants incubated with 300 μg/ml free GT and 1.8 μM unconjugated (hydrolyzed) TR exhibited negligible fluorescence in the sensory epithelium (FIG. 11C), as did explants incubated with unconjugated TR alone (FIG. 11D). Thus, because GTTR fluorescence in saccular explants was reduced by excess free GT and was not replicated by free Texas Red, the fluorescence distribution pattern in explants treated with GT/GTTR was considered representative of GTTR localization.

Distribution of GTTR in Bullfrog Saccular Explants

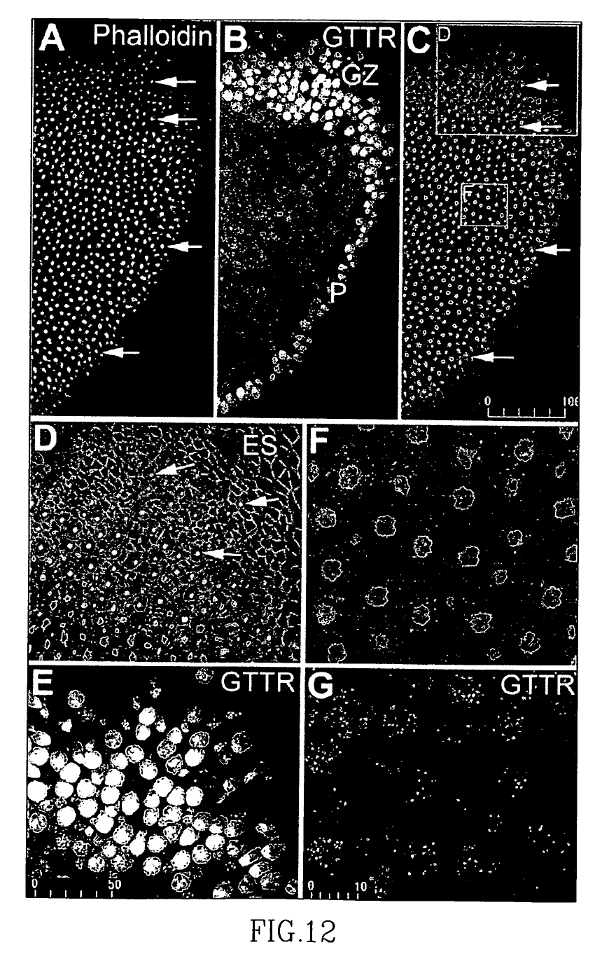

To identify the cell type(s) preferentially accumulating GTTR at the saccular periphery, explants were incubated with 300 μg/ml GT/GTTR for 30 minutes, prior to fixation, permeabilization and labeling for filamentous actin with FITC-phalloidin. Intense FITC-phalloidin labeling revealed a kidney-shaped region of bright dots resembling the extent of the sensory epithelium, and a reticulated network outlining cells throughout the epithelial sheet (FIG. 12A). The bright dots represent the hair cell bundles viewed from above. GTTR fluorescence occurred throughout the sensory epithelium and particularly at its periphery (FIG. 12B). Super-imposition of the FITC-phalloidin and GTTR fluorescence signals revealed that the hair bundles of the sensory epithelium were further outlined by a perimeter of intense red fluorescent cells, outside of which there was little GTTR fluorescence (FIGS. 12C,D). At higher magnification, the peripheral red fluorescent cells in the growth zone regions at the neural edge of the macula (FIGS. 12B,D) and around the periphery of the sensory macula can be identified as hair cells, indicated by the FITC-labeled hair bundle, within a circular cell apex surrounded by supporting cells with green polygonal margins (Steyger et al. 1997). The red fluorescent signal (FIG. 12D; shown monochromatically in FIG. 12E) revealed minimal GTTR fluorescence in the supporting cells surrounding the fluorescent hair cells. Within the peripheral hair cells, GTTR fluorescence was punctate and also diffusely dispersed throughout the elongated cell body (FIG. 12E). Hair cells with elongated cell bodies have been characterized as immature hair cells (Lewis 1985; Baird et al. 1996; Steyger et al. 1997). Within the central region of the sensory epithelium, large cells with circular apices exhibited less intense, punctate GTTR fluorescence than peripheral hair cells, together with diffuse somatic fluorescence not present in adjacent cells (FIG. 12G). The large rotund cells displayed FITC-phalloidin labeling of a circular cell apex, from which a labeled hair bundle protrudes perpendicular to the surface of the sensory epithelium (FIG. 12F), characteristic of mature hair cells (Lewis 1985; Baird et al. 1996; Steyger et al. 1997). These mature hair cells were typically surrounded by polygonal supporting cells with negligible GTTR fluorescence (FIGS. 12F,G).

Comparison of GTTR with Immunolabeled Gentamicin Distributions

The distribution of GTTR fluorescence was compared to the distribution pattern of unconjugated gentamicin revealed by indirect immunofluorescence. In low power images, explants incubated with 300 μg/ml GT/GTTR for 30 minutes displayed typical GTTR fluorescence throughout the sensory epithelium and preferentially at the periphery (FIG. 13A), as described earlier. After incubation with GT/GTTR for 2 hours, the difference in the intensity of fluorescence between the peripheral and central hair cell zones was substantially reduced (FIG. 13B).

Explants incubated with unconjugated GT for 30 minutes or 2 hours, detected using gentamicin immunocytochemistry, revealed immunolabeling throughout the sensory macula, with only a slight preferential increase in fluorescence at the periphery (FIGS. 13, C,D). Explants incubated in normal culture media without GT for 30 minutes had negligible fluorescence following incubation with GT antisera and secondary antibodies (FIG. 13E). Explants incubated with unconjugated GT for 30 minutes, fixed and immunolabeled with GT-adsorbed primary antibodies, revealed negligible labeling compared to positively-labeled explants (FIG. 13F).

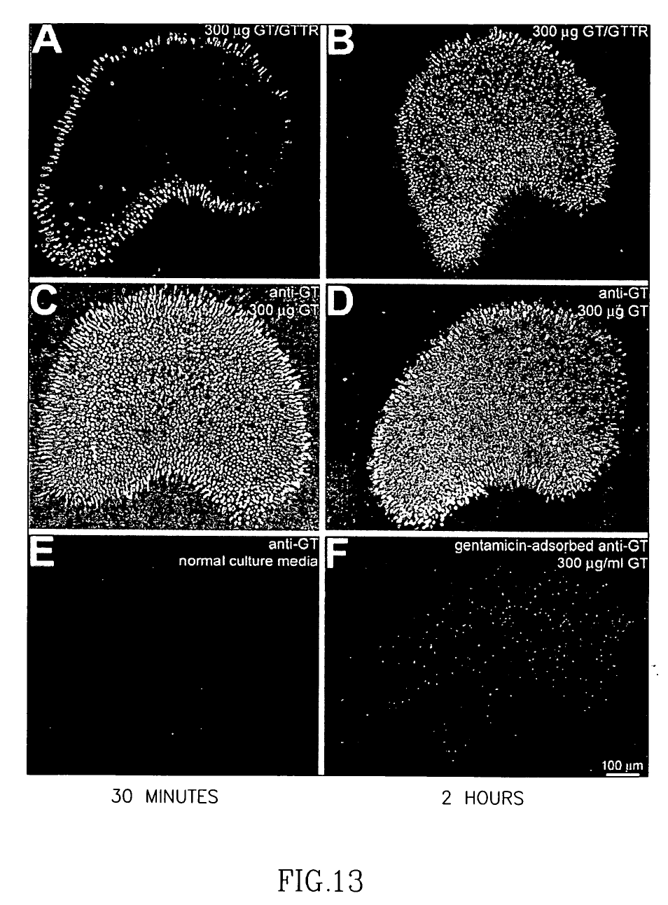

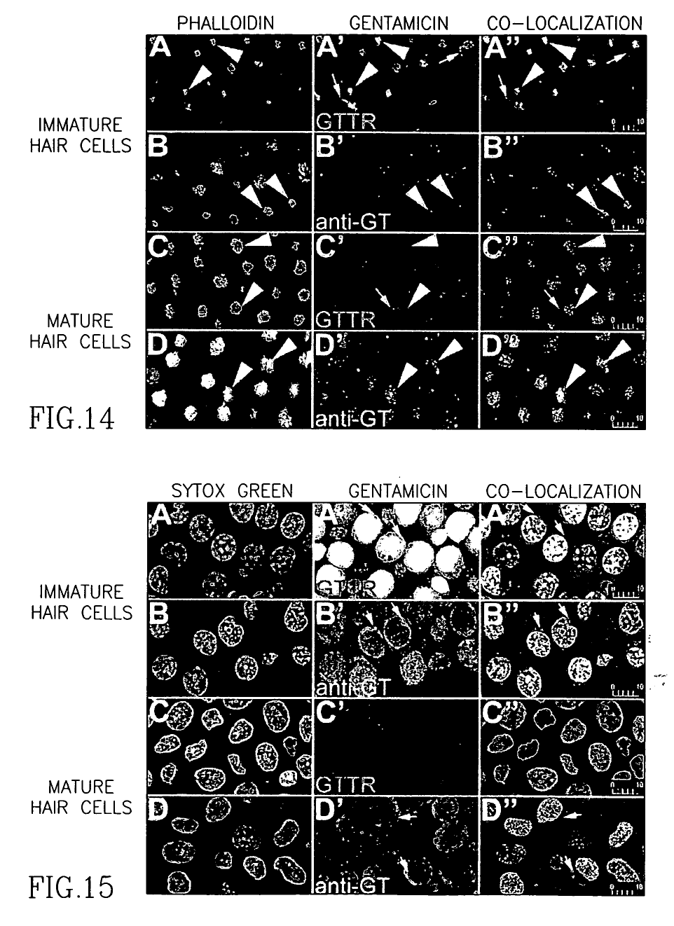

The distributions of GTTR and immunolabeled GT fluorescence in hair bundles were compared with phalloidin-Alexa-660 labeling in explants incubated with GTTR or unconjugated GT for 30 minutes. Both GTTR and immunolabeled GT fluorescence were identified in the vicinity of phalloidin-labeled hair bundles of both mature and immature hair cells (FIG. 14). Co-localization analysis revealed numerous pixels within hair bundles that contained both red (GTTR or immunolabeled gentamicin) and blue (phalloidin-Alexa-660) intensities above a user-defined threshold, confirming that phalloidin-labeled stereocilia were labeled with either GTTR or GT antibodies. The kinocilium of several immature and mature hair cells also exhibited GTTR fluorescence (FIG. 14). No cross-talk or bleed-through of Alexa-660-phalloidin fluorescence could be determined in the Texas Red channel (or vice versa) at the same laser power and acquisition settings used to collect stereociliary images.

The distributions of GTTR and immunolabeled GT fluorescence in hair cell nuclei were also compared in explants incubated with GTTR or unconjugated GT for 30 minutes, and subsequently labeled with Sytox Green that is specific for nucleic acids (FIG. 15). At the saccular periphery, GTTR and immunolabeled GT were both present within immature hair cell nuclei (FIGS. 15A″, B″). Co-localization analysis revealed many pixels in immature hair cell nuclei that contained GTTR or immunolabeled GT fluorescence at intensities above a user-defined threshold, confirming that these nuclei were labeled with GTTR or GT antibodies (FIG. 15A″, B″). In mature hair cells, only immunolabeled GT could be readily identified in the nuclei (FIG. 15D″). No cross-talk of Sytox Green fluorescence could be determined in the Texas Red channel at the same laser power and acquisition settings used to collect these images.

Subcellular Compartmentalization of GTTR

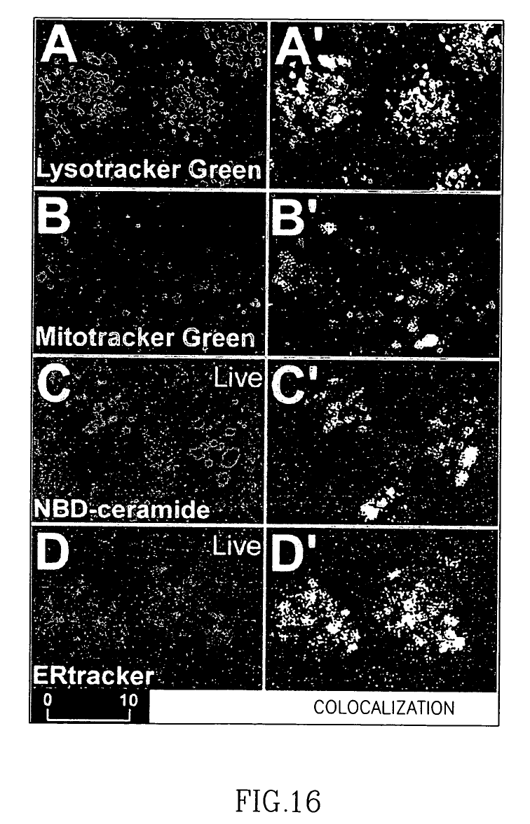

Live saccular explants pre-loaded with Lysotracker Green, Mitotracker Green, NBD-ceramide, and ERtracker were treated with GTTR for 2 hours, prior to washout, and imaging. Co-localization analysis (FIGS. 16A′-D′) revealed that the pixel clusters of punctate (red) GTTR fluorescence were co-localized with clusters of green pixels generated by fluorescence emission of Lysotracker Green, Mitotracker Green, NCB-ceramide, and ERtracker. The resolution of the confocal microscopy system (>230 nm, ×60 objective, N.A. 1.4) demonstrates that GTTR fluorescence is in the vicinity of these organelles.

Ultrastructural Localization of Gentamicin

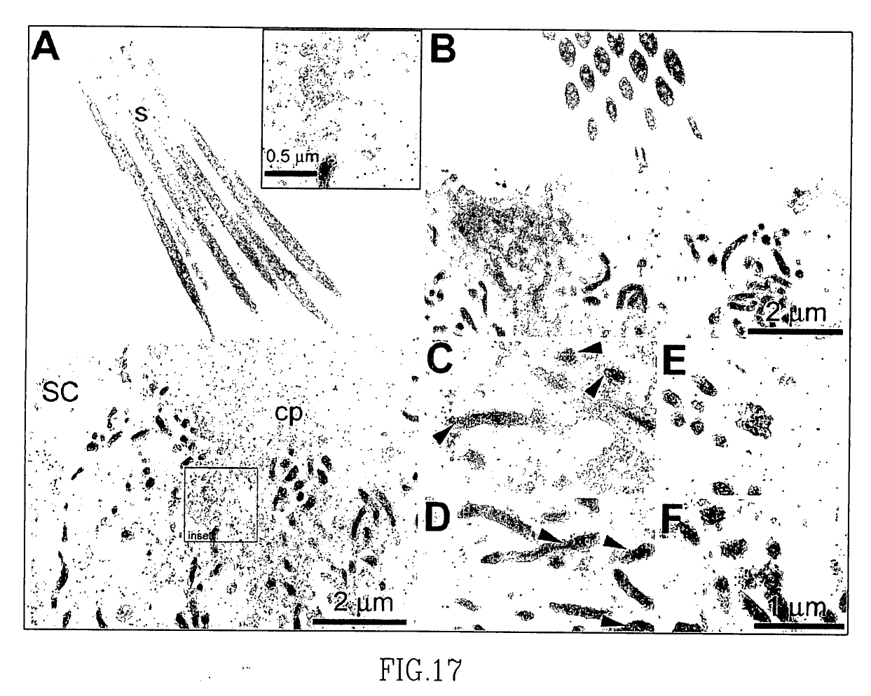

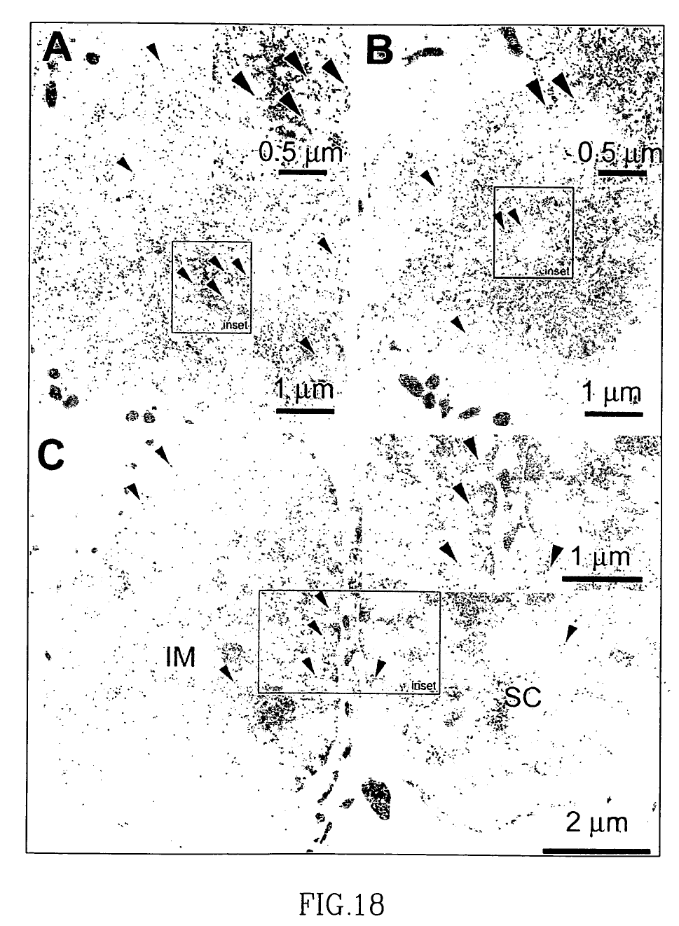

On LR Gold sections, GT immunogold labeling was observed in the vicinity of the hair bundle and at the endolymphatic membrane of hair cells. Labeling was also observed within endosomes and other vesicular structures in the infra-cuticular regions of all hair cell types, adjacent to and within mitochondria, and within the nucleus (FIGS. 17, 18). Often, gold particles could not be assigned to a particular structure, and yet were present in significant numbers in the hair cell cytoplasm (FIG. 17A, and inset; 17C, D, E). No qualitative differences in the distribution of gold labeling could be determined at this ultrastructural level between mature and immature hair cells. The cytoplasmic immunogold labeling seen within hair cells could not be observed in adjacent supporting cells (FIG. 17A), nor in hair cells from explants not incubated with GT (FIG. 17B). Thus, this cytoplasmic labeling may be the ultrastructural equivalent of the diffuse fluorescent labeling seen in hair cells (FIG. 12). Supporting cells also displayed comparatively weak gold labeling in the nucleus compared to hair cell nuclei (FIG. 18C). Control sections of explants incubated with GT, and immuno-processed without primary antibodies, or with GT-adsorbed GT antibodies, displayed negligible gold labeling. Sections of control explants (without GT treatment) incubated in the presence of GT antisera and secondary antibodies also displayed negligible non-specific labeling (FIG. 17B), except for very weak non-specific labeling in the hair cell nucleus (FIG. 18B, compared to gentamicin-treated explants; FIG. 18A).

Distribution of GTTR Fluorescence

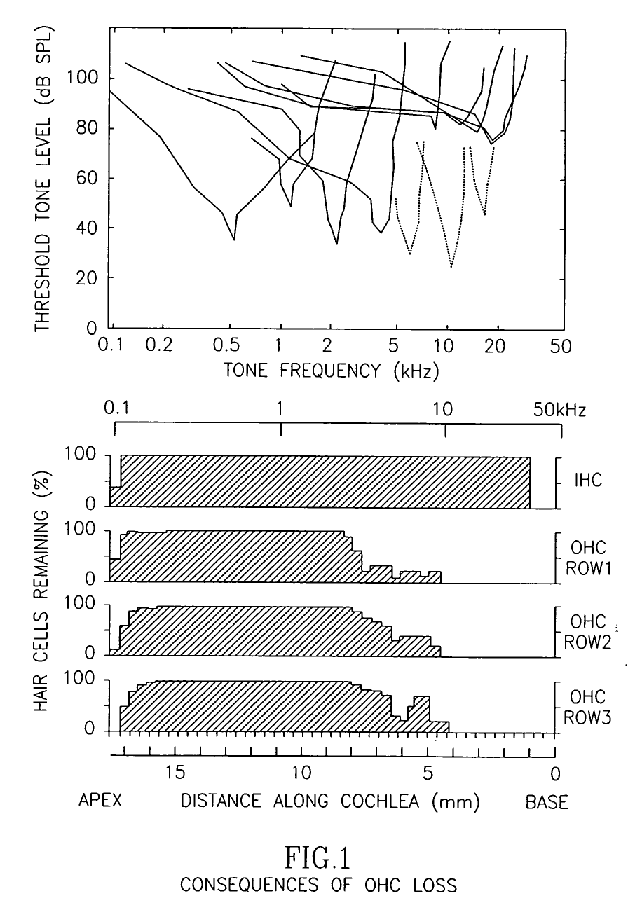

Negligible fluorescence in confocal images of explants incubated with unconjugated Texas Red (in the presence or absence of unconjugated gentamicin) demonstrates the specificity of the fluorescence as originating from the gentamicin-Texas Red conjugate (GTTR), rather than the Texas Red molecule. These in vitro studies used constant gentamicin levels as in previous in vitro studies and direct intra-otic gentamicin injections of bullfrogs (Baird et al. 1993; Baird et al. 1996; Steyger et al. 1997). However, these in vitro and intra-otic methods invoke higher aminoglycoside concentrations in inner ear fluids than those achieved following systemic injections in vivo, where aminoglycoside accumulation in inner ear fluids vary as a function of access, time and serum clearance (Tran Ba Huy et al. 1981). There is a possibility that this constant (higher) concentration of gentamicin in culture media affects the route(s) by which GTTR enters hair cells in vitro, which can be determined. If 300 μg/ml gentamicin (<0.5 mM) in culture media were to induce membrane rupture and artifactual entry of both gentamicin and GTTR, then much higher concentrations of gentamicin should increase that effect. However, decreased fluorescence in explants incubated with GT/GTTR mixture plus an additional 12 mg/ml gentamicin (FIG. 1B) contra-indicate this possibility.

The distribution of GTTR fluorescence in saccular explants is remarkably similar to that observed for immunolabeled gentamicin, at both light and EM levels. The fluorescence of GTTR was also only slightly more intense in peripheral hair cells than in mature hair cells after exposure to gentamicin for 2 hours (as was the fluorescence of immunolabeled gentamicin after 30 minutes or 2 hours). The qualitatively more intense punctate GTTR fluorescence in peripheral hair cells compared to mature hair cells at early time points may be due to increased endocytotic activity in peripheral hair cells, as observed in other explant protocols (Stanislawski et al. 1997). Indeed, the preferential accumulation of GTTR by peripheral hair cells is not replicated in in vivo studies following systemic injection (Dai et al., 2003). In these studies using GTTR, there was always a 300-fold excess of unlabeled GT. This may have resulted in competition between gentamicin and GTTR for binding sites, transporters, or ion channels which would not occur in GT-only treated explants. In fact, in these immunocytochemical experiments, all the gentamicin is available for immunodetection.

Aminoglycosides are used routinely to block the mechano-electrical transduction channel (Denk et al. 1992). Thus, the presence of GTTR and immunolabeled gentamicin at the location of the hair bundle of mature and immature hair cells is unsurprising, corroborating previous reports (Tachibana et al. 1985; Tachibana et al. 1986; Richardson et al. 1989). These ultrastructural studies are not able to distinguish fluorescent or immunogold labeling binding to glycocalyceal or membraneous structures of the hair bundle (Au et al. 1987; Marche et al. 1987; Richardson et al. 1989) from binding within the stereocilia.

Mature hair cell nuclei were also strongly immunolabeled for gentamicin, but only weakly labeled by GTTR. This may be a function of molecular size limiting the passage of GTTR (compared to unconjugated gentamicin) through the cytoplasm and through nuclear pores in mature hair cells, however, immature hair cell nuclei are strongly labeled by GTTR. Mature hair cells may have greater cytoplasmic affinity for GTTR (because of their larger volume), or ability to sequester GTTR in vesicles, compared to immature hair cells, thereby reducing passage of GTTR to the nucleus. If so, this may limit the utility of the GTTR conjugate. Alternatively, the stronger presence of immunolabeled gentamicin in mature hair cell nuclei, compared to GTTR at early time points, may be due to competition between GTTR and unlabeled gentamicin for entry into the nuclei. Competition experiments between GTTR and unlabeled gentamicin during cellular accumulation is currently under investigation in this laboratory.

In the hair cell soma, GTTR is co-localized with fluorescence emissions of Lysotracker Green-, Mitotracker Green-, NBD-ceramide-, or ERtracker-labeled organelles in hair cells within 2 hours, suggesting that GTTR is accumulated by lysosomes, mitochondria, Golgi bodies, and ER after uptake, as in kidney cells (Sandoval et al. 1998; Sandoval et al. 2000). Although the resolution (>230 nm) of the confocal microscopy technique cannot confirm that GTTR is within the organelle membranes of these sub-compartments, GTTR is located at least in the vicinity of these sub-cellular membrane-bound structures.

Gentamicin immunogold labeling was localized on the stereocilia and within the cuticular plate. Labeling was also associated with membrane-bound vesicles in the infra-cuticular cytoplasm and mitochondria, and was distinctly above background levels within the nucleus. This confirms the patterns of GTTR and immunolabeled gentamicin fluorescence obtained using confocal microscopy. The overall GTTR (and immunolabeled) gentamicin distribution reported here also correspond closely with the previous localization of aminoglycosides in lysosomes, nuclei and mitochondria of hair cells in previous studies (de Groot et al. 1990; Ding et al. 1995, 1997; Hashino et al. 1997).

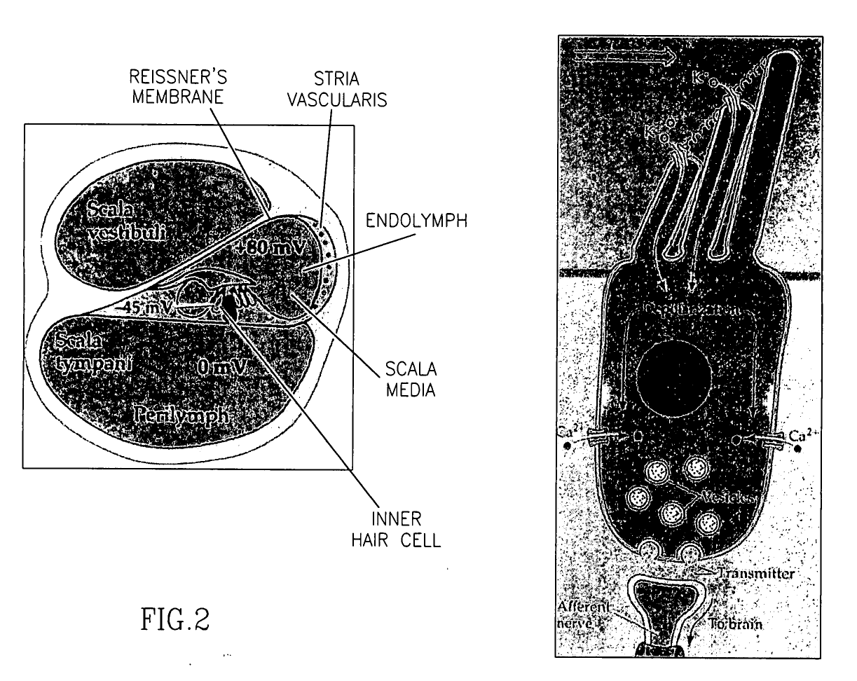

The distribution of gentamicin within hair cells is not solely confined to subcellular compartments like mitochondria and lysosomes. A significant fraction of GTTR and gentamicin immunolabeling was diffusely distributed throughout the cytoplasm (FIGS. 2, 5A′, A″), and unassociated with particular structures in post-embedding immunoelectron microscopy (FIG. 7), confirming previous reports of cytoplasmic labeling in kidney proximal tubules, retinal neurons, and guinea pig organ of Corti (Wedeen et al. 1983; Tabatabay et al. 1990; Beauchamp et al. 1991). FIG. 6, illustrating the co-localization of GTTR with fluorescently labeled organelles, shows an apparent lack of cytoplasmic GTTR labeling. This may be due to the intense fluorescence of the organelle-associated GTTR overwhelming the cytoplasmic labeling. In addition, explants used in FIG. 6 were not solvent-permeabilized, as were explants used in FIGS. 2-5. We have recently found that solvent treatment unquenches masked GTTR fluorescence through delipidation. Thus, the overall degree of correspondence between GTTR and immunolabeled gentamicin in these studies suggests that GTTR reaches the same intracellular locations as unconjugated gentamicin. In addition, these distributions largely agree with the distribution of aminoglycosides administered both systemically and in vitro in previous studies.

Consequences of Gentamicin Accumulation

The accumulation of gentamicin by a variety of subcellular organelles suggests a variety of interactions between aminoglycosides and eukaryote cells, ranging from interactions with ion channels/receptors, endocytotic uptake, to modulating intracellular chemical activities. Gentamicin promotes calcium influx via the calcium-sensing receptor (McLarnon et al. 2002; Ward et al. 2002). Aminoglycosides also block stereociliary mechanosensitive transduction channels and have recently been reported to enter hair cells via these same channels (Hudspeth 1982; Kroese and van den Bercken 1982; Hudspeth and Kroese 1983; Gale et al. 2001; Marcotti and Kros 2002). Entry of aminoglycosides into the cytoplasmic domain could facilitate accumulation by mitochondria and nuclei via diffusion or cytoplasmic trafficking, rather than by endosomal transport.

Aminoglycosides (and FM1-43) are thought to enter the cytoplasmic domain of hair cells through cation channels (Gale et al. 2001; Marcotti and Kros 2002; Meyers et al. 2003). Although the polycationic GTTR has a molecular weight 2.5-3 times greater than native gentamicin, other large organic molecules, e.g., tetrahexylammonium, YO-PRO, can pass through cation channels (Khakh et al. 1999; Virginio et al. 1999; Huang et al. 2000). Several characteristics other than molecular weight, for example: physical dimensions, charge, hydrophobicity, etc., also impact the ability of any specific molecule to permeate through any particular ion channel.

Receptor-mediated endocytosis is a major mechanism of gentamicin uptake in kidney cells, where megalin and cubulin potentially play significant roles (Moestrup et al. 1995; Christensen and Birn 2001). Endosomal trafficking of GTTR leads to the endoplasmic reticulum (ER), Golgi bodies, and lysosomes (Sandoval et al. 1998; Sandoval et al. 2000; Sundin et al. 2001). Lysosomal retention of aminoglycosides by surviving hair cells following treatment has been implicated in the continuing degeneration of hair cells following cessation of treatment (Aran et al. 1993; Dulon et al. 1993; Hashino et al. 2000), but whether this is the primary site of aminoglycoside toxicity remains unclear. If lysosomal lysis were the major mechanism of aminoglycoside toxicity, the release of lysosomal hydrolases should accelerate the rate of necrosis. However, chronic low-level exposure to aminoglycosides increases the rate of apoptosis of kidney proximal tubule cells, but not necrosis (El Mouedden et al. 2000a; El Mouedden et al. 2000b; Ward et al. 2002).

Gentamicin toxicity also induces intracellular oxidative stress, and the release of mitochondrial enzymes, including cytochrome C, that are powerful promoters of apoptosis (Deshmukh and Johnson 1998; Hirose et al. 1999; Sha and Schacht 1999a, b; Walker et al. 1999; Cheng et al. 2002). Thus, the association of gentamicin with mitochondria is not surprising. The mechanism(s) by which gentamicin induces functional changes in mitochondria still remains unclear. Nonetheless, loss of mitochondrial function will have severe ramifications in the highly metabolically-active hair cells, including oxidative stress, increase in free oxygen radicals, and loss of ATP production, all of which contribute to induction of apoptosis.

Nuclear accumulation of gentamicin suggests that gentamicin could directly interact with nuclear material rapidly after uptake. Several previous studies have shown nuclear uptake of aminoglycosides in both the ear and kidney (Nassberger et al. 1990; Beauchamp et al. 1991; Ding et al. 1995, 1997). However, other studies have reported neither the presence nor absence of gentamicin in the nucleus (Hashino et al. 1997; Sundin et al. 1997; Girton et al. 2002). Nonetheless, a subset of cystic fibrosis patients can be partially rehabilitated through gentamicin therapy, which causes by-passing of the premature stop codon in the cystic fibrosis (CF) mutation, allowing functional transcription of the CF transmembrane protein (Howard et al. 1996; Bedwell et al. 1997; Wilschanski et al. 2000; Clancy et al. 2001; Du et al. 2002; Zsembery et al. 2002). This suggests that gentamicin can enter the nucleus.

Although accumulation of gentamicin causes numerous cytochemical (Imamura and Adams 2003a), cytoskeletal (Hackney et al. 1990; Steyger 1991) and physiological changes (Staecker et al. 1996; Hirose et al. 1997; Hirose et al. 1999; McLarnon et al. 2002; Ward et al. 2002), the functional impact of aminoglycoside accumulation in hair cells still remains poorly understood. The data in this example show the validity of using fluorophore-conjugated gentamicin (GTTR) to characterize the intracellular distribution of gentamicin in fixed, wholemounted tissues and permits the acquisition of high-resolution, 3-dimensional data-sets, which is not possible with sectioned material.

### References for Example 6

Aran, J. M., Dulon, D., Hiel, H., Erre, J. P., Aurousseau, C. (1993) [Ototoxicity of aminosides: recent results on uptake and clearance of gentamycin by sensory cells of the cochlea]. Rev Laryngol 0 to 1 Rhinol 114: 125-8.
Au, S., Weiner, N. D., Schacht, J. (1987) Aminoglycoside antibiotics preferentially increase permeability in phosphoinositide-containing membranes: a study with carboxyfluorescein in liposomes. Biochim Biophys Acta 902: 80-6.
Baird, R. A., Steyger, P. S., Schuff, N. R. (1996) Mitotic and nonmitotic hair cell regeneration in the bullfrog vestibular otolith organs. Ann NY Acad Sci 781: 59-70.
Baird, R. A., Torres, M. A., Schuff, N. R. (1993) Hair cell regeneration in the bullfrog vestibular otolith organs following aminoglycoside toxicity. Hear Res 65: 164-74.
Barza, M., Lauermann, M. (1978) Why monitor serum levels of gentamicin? Clin Pharmacokinet 3: 202-15.
Beauchamp, D., Gourde, P., Bergeron, M. G. (1991) Subcellular distribution of gentamicin in proximal tubular cells, determined by immunogold labeling. Antimicrob Agents Chemother 35: 2173-9.
Bedwell, D. M., Kaenjak, A., Benos, D. J., Bebok, Z., Bubien, J. K., Hong, J., Tousson, A., Clancy, J. P., Sorscher, E. J. (1997) Suppression of a CFTR premature stop mutation in a bronchial epithelial cell line. Nat Med 3: 1280-4.
Cheng, A. G., Cunningham, L. L., Rubel, E. W. (2002) Translocation of Cytochrome c in Hair Cells of Gentamicin-Treated Avian Basilar Papilla. ARO Midwinter Meeting Abstracts 25: 134.
Christensen, E. I., Birn, H. (2001) Megalin and cubilin: synergistic endocytic receptors in renal proximal tubule. Am J Physiol Renal Physiol 280: F562-73.
Clancy, J. P., Bebok, Z., Ruiz, F., King, C., Jones, J., Walker, L., Greer, H., Hong, J., Wing, L., Macaluso, M., Lyrene, R., Sorscher, E. J., Bedwell, D. M. (2001) Evidence that systemic gentamicin suppresses premature stop mutations in patients with cystic fibrosis. Am J Respir Crit Care Med 163: 1683-92.
de Groot, J. C., Meeuwsen, F., Ruizendaal, W. E., Veldman, J. E. (1990) Ultrastructural localization of gentamicin in the cochlea. Hear Res 50: 35-42.
Denk, W., Keolian, R. M., Webb, W. W. (1992) Mechanical response of frog saccular hair bundles to the aminoglycoside block of mechanoelectrical transduction. J Neurophysiol 68: 927-32.
Deshmukh, M., Johnson, E. M., Jr. (1998) Evidence of a novel event during neuronal death: development of competence-to-die in response to cytoplasmic cytochrome c. Neuron 21: 695-705.
Ding, D., Jin, X., Zhao, J. (1995) [Accumulation sites of kanamycin in cochlear basal membrane cells]. Zhonghua Er Bi Yan Hou Ke Za Zhi 30: 323-5.
Ding, D., Jin, X., Zhao, J. (1997) [Accumulation sites of kanamycin in the organ of Corti by microautoradiography]. Zhonghua Er Bi Yan Hou Ke Za Zhi 32: 348-9.
Du, M., Jones, J. R., Lanier, J., Keeling, K. M., Lindsey, J. R., Tousson, A., Bebok, Z., Whitsett, J. A., Dey, C. R., Colledge, W. H., Evans, M. J., Sorscher, E. J., Bedwell, D. M. (2002) Aminoglycoside suppression of a premature stop mutation in a Cftr−/− mouse carrying a human CFTR-G542X transgene. J Mol Med 80: 595-604.
Dulon, D., Hiel, H., Aurousseau, C., Erre, J. P., Aran, J. M. (1993) Pharmacokinetics of gentamicin in the sensory hair cells of the organ of Corti: rapid uptake and long term persistence. C R Acad Sci III 316: 682-7.
El Mouedden, M., Laurent, G., Mingeot-Leclercq, M. P., Taper, H. S., Cumps, J., Tulkens, P. M. (2000a) Apoptosis in renal proximal tubules of rats treated with low doses of aminoglycosides. Antimicrob Agents Chemother 44: 665-75.
El Mouedden, M., Laurent, G., Mingeot-Leclercq, M. P., Tulkens, P. M. (2000b) Gentamicin-induced apoptosis in renal cell lines and embryonic rat fibroblasts. Toxicol Sci 56: 229-39.
Gale, J. E., Marcotti, W., Kennedy, H. J., Kros, C. J., Richardson, G. P. (2001) FM1-43 dye behaves as a permeant blocker of the hair-cell mechanotransducer channel. J Neurosci 21: 7013-25.
Girton, R. A., Sundin, D. P., Rosenberg, M. E. (2002) Clusterin protects renal tubular epithelial cells from gentamicin-mediated cytotoxicity. Am J Physiol Renal Physiol 282: F703-9.
Giuliano, R. A., Paulus, G. J., Verpooten, G. A., Pattyn, V. M., Pollet, D. E., Nouwen, E. J., Laurent, G., Carlier, M. B., Maldague, P., Tulkens, P. M., et al. (1984) Recovery of cortical phospholipidosis and necrosis after acute gentamicin loading in rats. Kidney Int 26: 838-47.
Gratacap, B., Charachon, R., Stoebner, P. (1985) Results of an ultrastructural study comparing stria vascularis with organ of Corti in guinea pigs treated with kanamycin. Acta Otolaryngol (Stockh) 99: 339-42.
Hackney, C. M., Furness, D. N., Steyger, P. S. (1990) Structural abnormalities in inner hair cells following kanamycin-induced outer hair cell loss. Mechanics and Biophysics of Hearing. P Dallos, C D Geisler, J W Matthews, M Ruggero and C R Steele, eds,: 10-17.
Hashino, E., Shero, M. (1995) Endocytosis of aminoglycoside antibiotics in sensory hair cells. Brain Res 704: 135-40.
Hashino, E., Shero, M., R. J., R. J. S. (1998) In vivo and in vitro assessment of lysosomal activity during kanamycin uptake in hair cells. ARO Midwinter Meeting Abstracts 24: #240.
Hashino, E., Shero, M., Salvi, R. J. (1997) Lysosomal targeting and accumulation of aminoglycoside antibiotics in sensory hair cells. Brain Res 777: 75-85.
Hashino, E., Shero, M., Salvi, R. J. (2000) Lysosomal augmentation during aminoglycoside uptake in cochlear hair cells. Brain Res 887: 90-7.
Hawkins, J. E., Jr., Johnsson, L. G., Aran, J. M. (1969) Comparative tests of gentamicin ototoxicity. J Infect Dis 119: 417-31.
Hirose, K., Hockenbery, D. M., Rubel, E. W. (1997) Reactive oxygen species in chick hair cells after gentamicin exposure in vitro. Hear Res 104: 1-14.
Hirose, K., Westrum, L. E., Stone, J. S., Zirpel, L., Rubel, E. W. (1999) Dynamic studies of ototoxicity in mature avian auditory epithelium. Ann NY Acad Sci 884: 389-409.
Howard, M., Frizzell, R. A., Bedwell, D. M. (1996) Aminoglycoside antibiotics restore CFTR function by overcoming premature stop mutations. Nat Med 2: 467-9.
Huang, C. J., Favre, I., Moczydlowski, E. (2000) Permeation of large tetra-alkylammonium cations through mutant and wild-type voltage-gated sodium channels as revealed by relief of block at high voltage. J Gen Physiol 115: 435-54.
Hudspeth, A. J. (1982) Extracellular current flow and the site of transduction by vertebrate hair cells. J Neurosci 2: 1-10.
Hudspeth, A. J., Kroese, A. B. A. (1983) Voltage-dependent interaction of dihydrostreptomycin with the transduction channels in bullfrog sensory hair cells. Journal of Physiology 345: 66P.
Humes, H. D. (1988) Aminoglycoside nephrotoxicity [clinical conference]. Kidney Int 33: 900-11.
Humes, H. D. (1999) Insights into ototoxicity. Analogies to nephrotoxicity. Ann NY Acad Sci 884: 15-8.
Imamura, S. I., Adams, J. C. (2003a) Changes in Cytochemistry of Sensory and Nonsensory Cells in Gentamicin-Treated Cochleas. J Assoc Res Otolaryngol (in press) First Online JARO website.
Imamura, S. I., Adams, J. C. (2003b) Distribution of Gentamicin in the Guinea Pig Inner Ear after Local or Systemic Application. J Assoc Res Otolaryngol (in press), First Online JARO web site.
Khakh, B. S., Bao, X. R., Labarca, C., Lester, H. A. (1999) Neuronal P2X transmitter-gated cation channels change their ion selectivity in seconds. Nat Neurosci 2: 322-30.
Kroese, A. B., van den Bercken, J. (1982) Effects of ototoxic antibiotics on sensory hair cell functioning. Hear Res 6: 183-97.
Lerner, S. A., Matz, G. J. (1979) Suggestions for monitoring patients during treatment with aminoglycoside antibiotics. Otolaryngol Head Neck Surg 87: 222-8.
Lewis, E. R., Leverenz, E. L. & Bialek, W. S. 1985. The vertebrate inner ear CRC Press, Boca Raton.
Marche, P., Olier, B., Girard, A., Fillastre, J. P., Morin, J. P. (1987) Aminoglycoside-induced alterations of phosphoinositide metabolism. Kidney Int 31: 59-64.
Marcotti, W., Kros, C. J. (2002) Dihydrostreptomycin is a Permeant Blocker of the Outer Hair Cell Transducer Channel. ARO Midwinter Meeting Abstracts 25: #602.
McLarnon, S., Holden, D., Ward, D., Jones, M., Elliott, A., Riccardi, D. (2002) Aminoglycoside antibiotics induce pH-sensitive activation of the calcium-sensing receptor. Biochem Biophys Res Commun 297: 71-7.
Meyers, J. R., MacDonald, R. B., Duggan, A., Lenzi, D., Standaert, D. G., Corwin, J. T., Corey, D. P. (2003) Lighting up the senses: FM1-43 loading of sensory cells through nonselective ion channels. J Neurosci 23: 4054-65.
Miller, J. J. 1985. Handbook of ototoxicity CRC Press, Boca Raton.
Moestrup, S. K., Cui, S., Vorum, H., Bregengard, C., Bjorn, S. E., Norris, K., Gliemann, J., Christensen, E. I. (1995) Evidence that epithelial glycoprotein 330/megalin mediates uptake of polybasic drugs. J Clin Invest 96: 1404-13.
Molitoris, B. A. (1997) Cell biology of aminoglycoside nephrotoxicity: newer aspects. Curr Opin Nephrol Hypertens 6: 384-8.
Nassberger, L., Bergstrand, A., DePierre, J. W. (1990) Intracellular distribution of gentamicin within the rat kidney cortex: a cell fractionation study. Exp Mol Pathol 52: 212-20.
Pawley, J. B. 1995. Handbook of biological confocal microscopy. 3rd ed. Plenum Press, New York and London.
Richardson, G. P., Russell, I. J., Wasserkort, R., Hans, M. (1989) Aminoglycoside antibiotics and lectins cause irreversible increases in the stiffness of cochlear hair-cell stereocilia. In: Wilson, J. P., Kemp, D. T., (Eds.) Cochlear Mechanisms—Structure, Function and Models. Plenum Press, New York. pp. 578-66.
Sandoval, R., Leiser, J., Molitoris, B. A. (1998) Aminoglycoside antibiotics traffic to the Golgi complex in LLC-PK1 cells. J Am Soc Nephrol 9: 167-74.
Sandoval, R. M., Dunn, K. W., Molitoris, B. A. (2000) Gentamicin traffics rapidly and directly to the Golgi complex in LLC-PK(1) cells. Am J Physiol Renal Physiol 279: F884-90.
Sha, S. H., Schacht, J. (1999a) Formation of reactive oxygen species following bioactivation of gentamicin. Free Radic Biol Med 26: 341-7.
Sha, S. H., Schacht, J. (1999b) Stimulation of free radical formation by aminoglycoside antibiotics. Hear Res 128: 112-8.
Staecker, H., Kopke, R., Malgrange, B., Lefebvre, P., Van de Water, T. R. (1996) NT-3 and/or BDNF therapy prevents loss of auditory neurons following loss of hair cells. Neuroreport 7: 889-94.
Stanislawski, L., Carreau, J. P., Pouchelet, M., Chen, Z. H., Goldberg, M. (1997) In vitro culture of human dental pulp cells: some aspects of cells emerging early from the explant. Clin Oral Investig 1: 131-40.
Steyger, P. S. (1991) Ultrastructural and immunohistochemical studies of cytoskeletal features in the guinea pig organ of Corti. Ph.D. Thesis, Keele University, U.K.
Steyger, P. S., Burton, M., Hawkins, J. R., Schuff, N. R., Baird, R. A. (1997) Calbindin and parvalbumin are early markers of non-mitotically regenerating hair cells in the bullfrog vestibular otolith organs. Int J Dev Neurosci 15: 417-32.
Steyger, P. S., Gillespie, P. G., Baird, R. A. (1998) Myosin Ibeta is located at tip link anchors in vestibular hair bundles. J Neurosci 18: 4603-15.
Sundin, D. P., Meyer, C., Dahl, R., Geerdes, A., Sandoval, R., Molitoris, B. A. (1997) Cellular mechanism of aminoglycoside tolerance in long-term gentamicin treatment. Am J Physiol 272: C1309-18.
Sundin, D. P., Sandoval, R., Molitoris, B. A. (2001) Gentamicin inhibits renal protein and phospholipid metabolism in rats: implications involving intracellular trafficking. J Am Soc Nephrol 12: 114-23.
Tabatabay, C. A., Young, L. H., D'Amico, D. J., Kenyon, K. R. (1990) Immunocytochemical localization of gentamicin in the rabbit retina following intravitreal injection. Arch Ophthalmol 108: 723-6.
Tachibana, M., Morioka, H., Machino, M., Amagai, T., Mizukoshi, O. (1986) Aminoglycoside binding sites in the cochlea as revealed by neomycin-gold labelling. Histochemistry 85: 301-4.
Tachibana, M., Morioka, H., Machino, M., Mizukoshi, O. (1985) Binding sites of an aminoglycoside in the cochlea examined by immunocytochemistry. Histochemistry 83: 237-40.
Tran Ba Huy, P., Manuel, C., Meulemans, A., Sterkers, O., Amiel, C. (1981) Pharmacokinetics of gentamicin in perilymph and endolymph of the rat as determined by radioimmunoassay. J Infect Dis 143: 476-86.
Virginio, C., MacKenzie, A., Rassendren, F. A., North, R. A., Surprenant, A. (1999) Pore dilation of neuronal P2X receptor channels. Nat Neurosci 2: 315-21.
Walker, P. D., Barri, Y., Shah, S. V. (1999) Oxidant mechanisms in gentamicin nephrotoxicity. Ren Fail 21: 433-42.
Ward, D. T., McLarnon, S. J., Riccardi, D. (2002) Aminoglycosides Increase Intracellular Calcium Levels and ERK Activity in Proximal Tubular OK Cells Expressing the Extracellular Calcium-Sensing Receptor. J Am Soc Nephrol 13: 1481-9.
Wedeen, R. P., Batuman, V., Cheeks, C., Marquet, E., Sobel, H. (1983) Transport of gentamicin in rat proximal tubule. Lab Invest 48: 212-23.
Wersall, J., Lundquist, P. G., Bjorkroth, B. (1969) Ototoxicity of gentamicin. J Infect Dis 119: 410-6.
Wilschanski, M., Famini, C., Blau, H., Rivlin, J., Augarten, A., Avital, A., Kerem, B., Kerem, E. (2000) A pilot study of the effect of gentamicin on nasal potential difference measurements in cystic fibrosis patients carrying stop mutations. Am J Respir Crit Care Med 161: 860-5.

Zsembery, A., Jessner, W., Sitter, G., Spirli, C., Strazzabosco, M., Graf, J. (2002) Correction of CFTR malfunction and stimulation of Ca-activated Cl channels restore HCO₃— secretion in cystic fibrosis bile ductular cells. Hepatology 35: 95-104.

TABLE 1
for Example 7: Theoretical and observed xy and z-axis resolution for
Bio-Rad MRC 1024 confocal system with a Nikon TE300 microscope.

| Oil | | 512 × 512 | | 1024 × 1024 | |
|---|---|---|---|---|---|
| immersion | | | Observed | | Observed |
| objective | | Pixel size | resolution | Pixel size | resolution |
| | Theoretical | | | | |
| | xy resolution^{a} | | | | |
| 40× (n.a. 1) | 257.5 nm | 474 nm | 490 nm | 237 nm | 350 nm |
| 40× + zoom | 257.5 nm | 200 nm | 280 nm | 100 nm | 260 nm |
| 60× (n.a. 1.4) | 184 nm | 316 nm | 330 nm | 158 nm | 230 nm |
| 60× + zoom | 184 nm | 100 nm | 230 mn | 100 nm | 230 nm |
| | Theoretical | | | | |
| | xz resolution^{b} | | | | |
| 40× + zoom | 390.1 nm | | | 100 nm | 600 nm |
| 60× + zoom | 199 nm | | | 100 nm | 440 nm |

Theoretical resolution formulae (Nikon):
^{a}for the xy resolution: (λ/(2 × N.A.)); ^{b}for the z-axis resolution ((λ × \acute{η})/2 × (N.A.)2) + (\acute{η}/7 × N.A. × mag).
where: λ, wavelength (515 nm); \acute{η}, immersion oil refractive index (1.515); N.A., numerical aperture; and mag, magnification.

### EXAMPLE 7

Vertebrate sensory hair cells are pharmacologically-sensitive to aminoglycoside antibiotics used in life-threatening Gram-negative bacterial infections, e.g. meningitis. The nephrotoxicity and ototoxicity of aminoglycosides are well-known, but the rate of aminoglycoside uptake in vivo remains poorly understood. Prior to the present invention, little was known about the rate of drug uptake in vivo. In this example, we found that fluorescently-conjugated gentamicin is preferentially taken up by hair cells 6-9 hours post-injection, in a cochleotopic gradient (from high-to-low frequency regions).

Vertebrate sensory hair cells are pharmacologically-sensitive to aminoglycoside antibiotics used in life-threatening Gram-negative bacterial infections, e.g. meningitis. The nephrotoxicity and ototoxicity of aminoglycosides are well-known, but the rate of aminoglycoside uptake in vivo remains poorly understood. Recent work has used Texas Red-conjugated gentamicin (GT-TR) to identify the intracellular locations of GT-TR within hair cells (nuclei, mitochondria, Golgi bodies, endoplasmic reticulum, and throughout the cytoplasm) following uptake by kidney and inner ear tissues in vitro (1-3).

Gentamicin sulfate was conjugated to Texas Red (Molecular Probes) using published methods (1) to produce a 300:1 molar dilution of gentamicin-Texas Red conjugate (GTTR). Mice were injected sub-cutaneously with a single 300 mg/kg dose of GTTR. Frogs were injected directly into the lymph sac (there is no sub-cutaneous space in frogs, lymph sacs drain directly into the blood). Control animals received an equivalent dose of hydrolyzed Texas Red succinimidyl esters only.

At specific time-points (0.5, 1, 2, 3, 6, 7.5, 9 and 24 hours) following injection, animals were anesthetized and inner ear organs were excised and fixed in 4% formaldehyde overnight. After washing in PBS, frog and murine organs were permeabilized using ice-cold acetone, and all organs were labeled with Alexa-488-conjugated, or FITC-conjugated phalloidin to localize filamentous actin. Mouse and frog inner ear epithelia were whole-mounted and observed using a Bio-Rad MRC 1024 ES laser scanning confocal system attached to a Nikon Eclipse TE300 inverted microscope. Alexa-488 and Texas Red images were collected sequentially using 1024×1024 pixel box size using a 4× (n.a. 0.25) and a 60× lens (n.a. 1.4) with an xy resolution=230 nm and xz resolution=440 nm. All control organs were imaged at the same laser intensity and gain settings as treated organs.

For data analysis of murine samples, area intensity measurements of optically-sectioned hair cells (approximately 1-2 microns below the cuticular plate) were obtained from apical and basal cochlear regions (25 hair cells per row in each region). The mean basal intensity was divided by the mean apical intensity of an equivalent number of hair cells to derive a ratio that indicates the relative degree of fluorescence in basal hair cells compared to apical hair cells. These ratios were plotted over time.

As shown in FIG. 19, bullfrog saccular immature hair cells accumulate less GTTR in vivo compared to explants in vitro. The adult bullfrog saccule typically has 2000-3000 mature hair cells, surrounded by a periphery of immature hair cells. Following in vivo injection, GTTR is most prominent in mature hair cells in the central region of the saccule. The immature hair cells, at the saccular periphery, are clearly less intensely labeled (*) than mature hair cells (HC) at early time points (15 mins, 30 mins, and 1 hour) (FIG. 19). This is in contrast to a perimeter of intense labeling in immature hair cells after in vitro treatment with GTTR (2). These peripheral hair cells are shown in the lower row of images as indicated. Even 24 hours after in vivo GTTR injection, GTTR uptake by immature hair cells remains relatively weak at the periphery of the saccule compared to mature hair cells. This in vivo pattern of GTTR uptake resembles the pattern of hair cell death in the bullfrog saccule, where the central more mature hair cells die more rapidly compared to the more resistant peripheral immature hair cells (4).

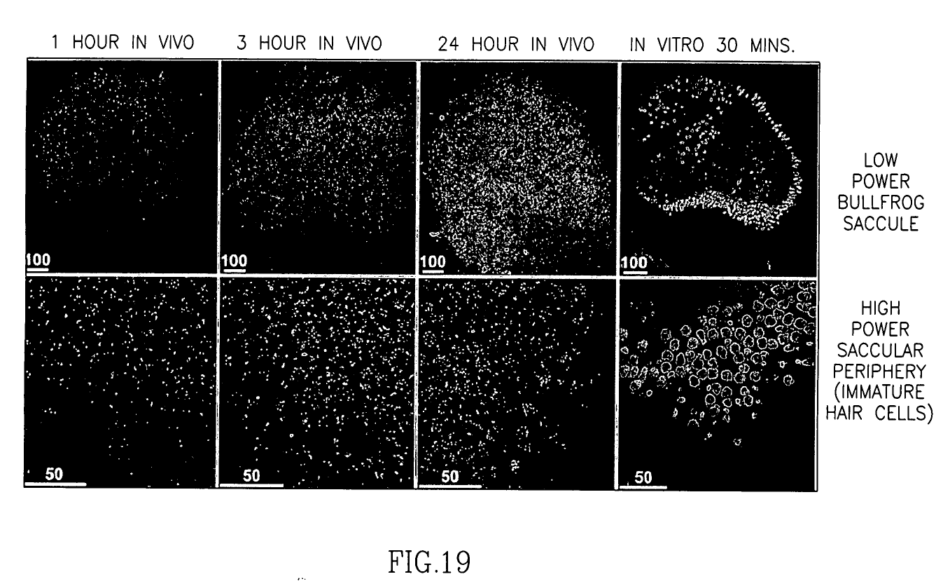

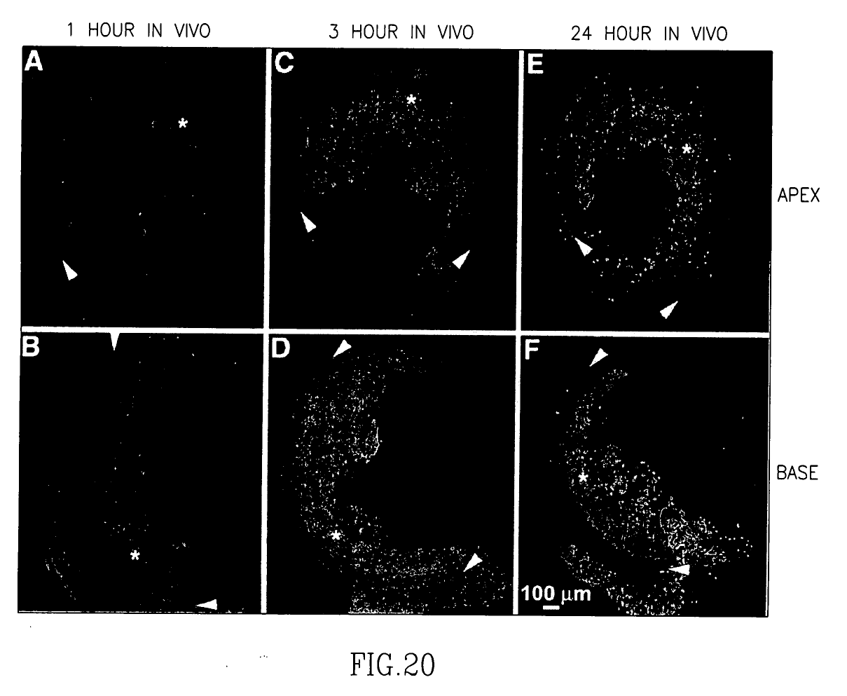

As shown in FIG. 20, GT-TR is more aggressively taken up at the base of the cochlea, under low power. Texas Red fluorescence in the organ of Corti (□) and spiral ganglion (*) in the basal coil is more intense compared to the same regions in the apical coil, at all time points. Spiral ganglion neurons are also pharmacologically sensitive to gentamicin toxicity (5).

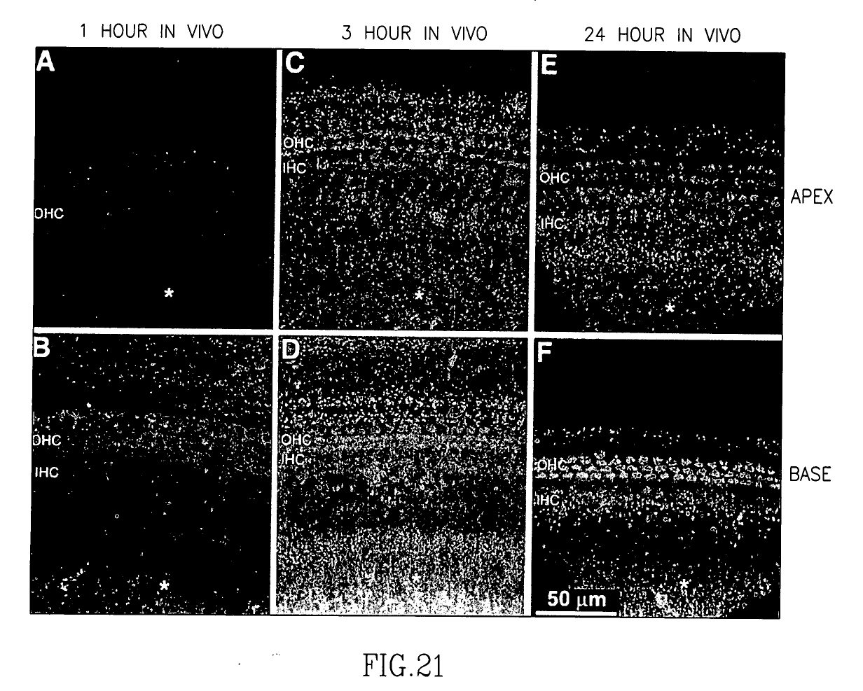

As shown in FIG. 21, GT-TR is more aggressively taken up at the base of the cochlea, under high power. Hair cells at the base of the cochlea display greater Texas Red fluorescence compared to apical hair cells at all time points. Outer hair cells (OHC, positioned over second row of OHC) at all time points in any region have greater fluorescence than inner hair cells (IHC). At 1 hour, fluorescence within hair cells appear relatively diffuse compared to later time points. There is relatively stronger fluorescence in the spiral ganglion area (*) of basal coils compared to apical coils in the same cochlea.

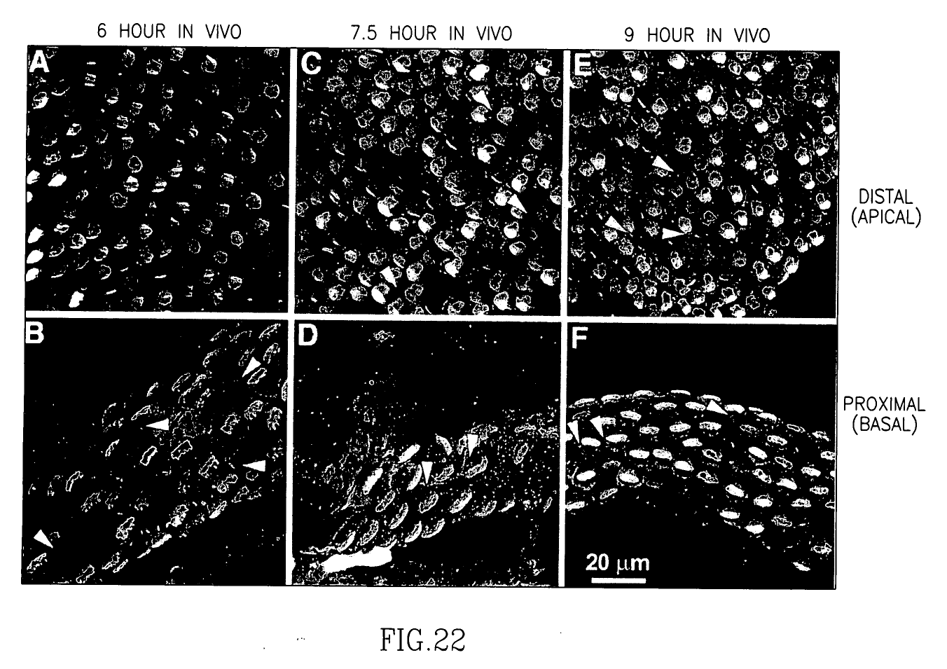

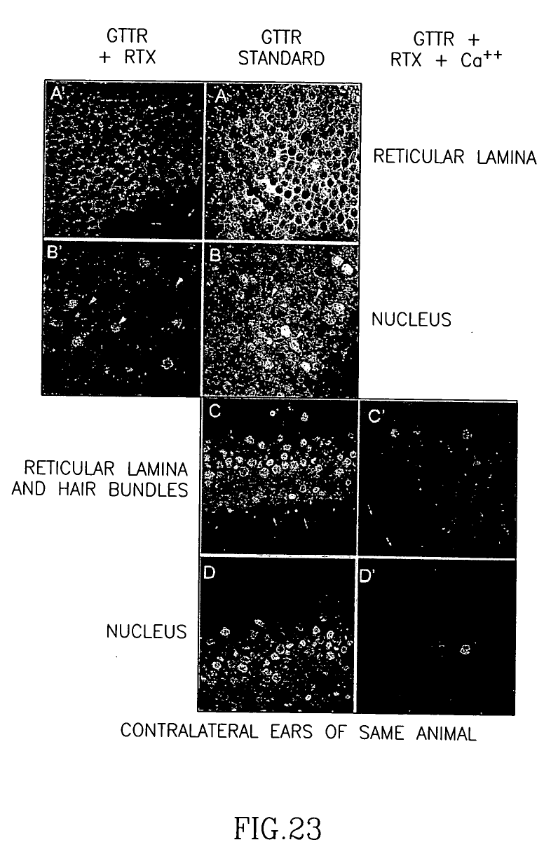

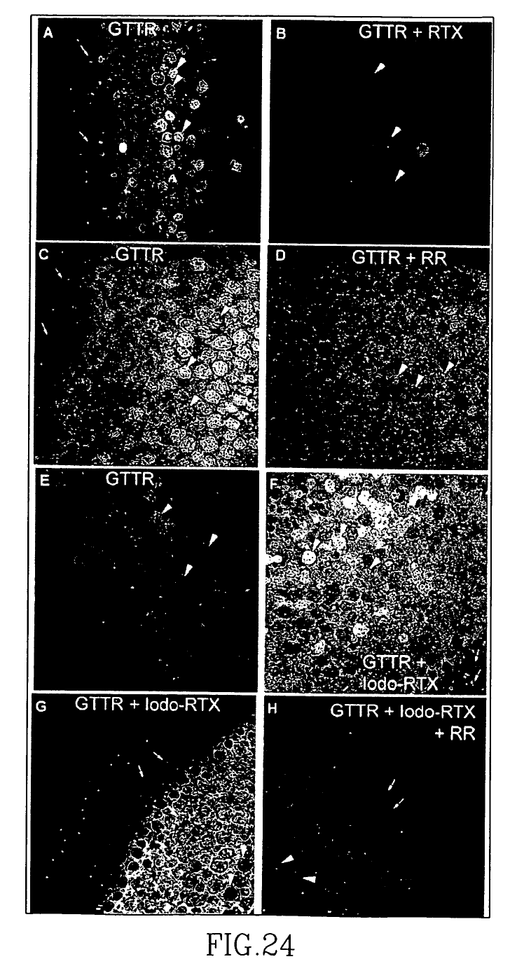

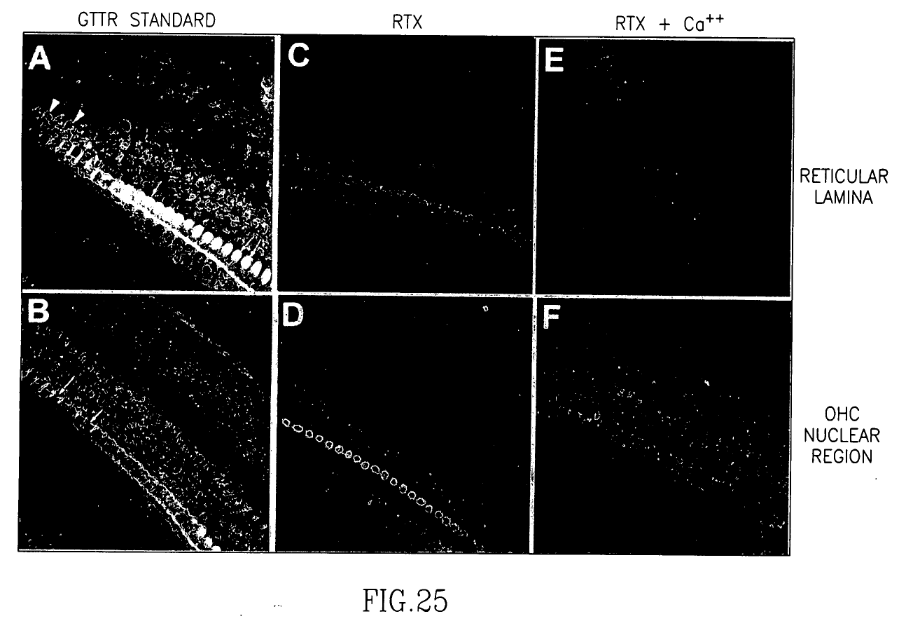

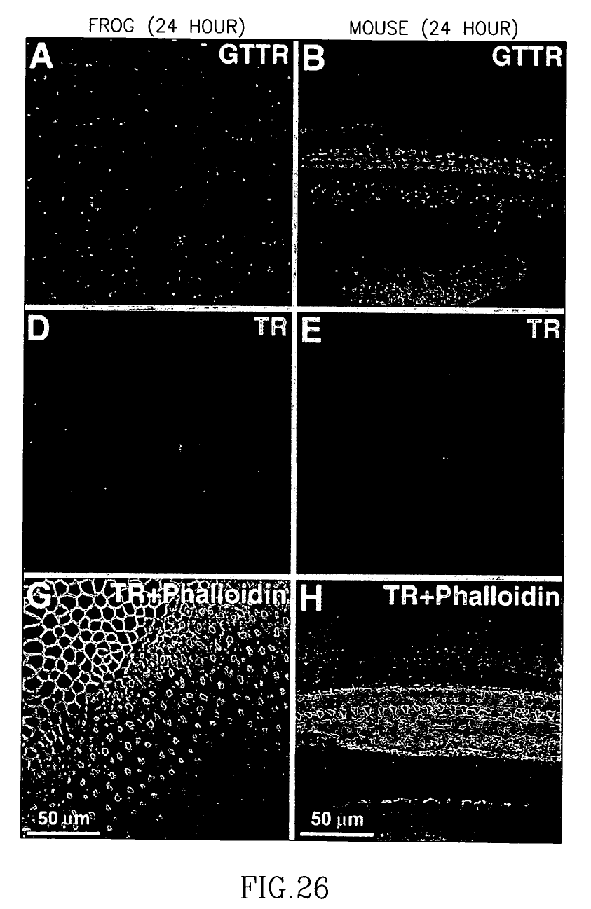

FIGS. 22 and 26 show the results of control experiments, in which GT-TR uptake patterns are not replicated by free Texas Red. Animals injected with free TR alone (FIG. 22) display negligible fluorescence in their inner ear epithelia at all time points. The typical distribution of GTTR in the (A) frog saccule and (B) mouse cochlea 24 hours after injection are shown (FIG. 26). Panels D and E show negligible TR fluorescence in the frog saccule and mouse cochlea, respectively, at equivalent time points. Panels G and H show actiniferous phalloidin labeling of the sensory epithelia in panels D and E, with the concomitant lack of red GTTR fluorescence.

As demonstrated in this example, vertebrate inner ear sensory hair cells accumulate GTTR in vivo following injection at distant sites. The pattern of GTTR uptake is similar to aminoglycoside-induced hair cell death in amphibians (4) and mammals (6).

In bullfrog peripheral saccular hair cells, the weak uptake of GTTR in vivo differs from the intense uptake of GTTR in vitro (2), indicating that accumulation of GTTR by immature hair cells in vitro is inconsistent with in vivo observations. However, uptake by mature hair cells appear comparable in both in vivo and in vitro environments.

The relatively-more rapid accumulation of GTTR by high frequency hair cells may be due to the larger number of stereocilia and transduction channels per hair cell at the base of the cochlea compared to hair cells at the apex of the cochlea. Indirect evidence suggests that gentamicin is able to enter hair cells via the transduction channel (8). GTTR accumulation is greatest at some distance away from the basal hook in mammalian cochleae, corroborating the onset position of drug-induced hair cell death in guinea pigs (9).

### References for Example 7

1. Sandoval R, Leiser J, Molitoris B A (1998) Aminoglycoside antibiotics traffic to the Golgi complex in LLC-PK1 cells. J Am Soc Nephrol 9:167-174.
2. Peters S, Hordichock A J, Steyger P S (2001) Fluorometric measurement of gentamicin uptake by bullfrog saccular hair cells. ARO Midwinter Meeting Abstracts 24:19622.
3. Hordichok A J, Peters S, Steyger P S (2000) Uptake of fluorophore-conjugated ototoxic drugs in sensory hair cells. ARO Midwinter Meeting Abstracts 26:#423.
4. Baird R A, Steyger P S, Schuff N R (1996) Mitotic and nonmitotic hair cell regeneration in the bullfrog vestibular otolith organs Ann NY Acad Sci 781:59-70.
5. Sone M, Schachern P A, Paparella M M (1998) Loss of spiral ganglion cells as primary manifestation of aminoglycoside ototoxicity Hear Res 115:217-223.
6. Hackney C M, Furness D N, Steyger P S (1990) Structural abnormalities in inner hair cells following kanamycin-induced outer hair cell loss. Mechanics and Biophysics of Hearing P Dallos, C D Geisler, J W Matthews, M Ruggero and C R Steele, eds, :10-17.
7. Cotanche D A, Lee K H, Stone J S, Picard D A (1994) Hair cell regeneration in the bird cochlea following noise damage or ototoxic drug damage Anat Embryol 189:1-18.
8. Gale J E, Marcotti W, Kennedy H J, Kros C J, Richardson G P (2001) FM1-43 dye behaves as a permeant blocker of the hair-cell mechanotransducer channel J Neurosci 21:7013-7025.
9. Tange R A, Conijn E A, van Zeijl L G, Huizing E H (1982) Pattern of gentamicin-induced cochlear degeneration in the guinea pig. A morphological and electrophysiological study Arch Otorhinolaryngol 236:173-184.

### EXAMPLE 8

### Quantitative and Microscopical Evidence for Non-Endocytotic Uptake of GTTR into the Cytoplasm and Nucleoli

A fluorescent microplate assay was developed using the MDCK kidney cell line to quantify the modulation of GTTR uptake by agonists/antagonists of the TRVP1 channel (pH, Ca²⁺, RTX, and iodo-RTX). These results confirm previous confocal microscopy observations that cold gentamicin reduced GTTR uptake, following incubation at either 37° C. for two hours or at room temperature for ten minutes (precluding endocytosis).

In addition, to verify the observed cytoplasmic and nuclear distributions of GTTR, gentamicin immunocytochemistry was performed on GTTR-treated and cold gentamicin-treated cells. Gentamicin immunolabeling was localized throughout the cytoplasm but was absent from the nucleolar-like structures within the nucleus for both treatments. However, GTTR co-localized with a RNA-specific fluorophore that label nucleolar-like structures in the nucleus. This suggests that the interaction between the gentamicin molecule and its binding site in the nucleolus is also interfering with the antigenic site, preventing nuclear-specific immunoreactivity.

This example demonstrates that microscopical observations of fluorescent gentamicin uptake by a rapid, bio-regulatory, non endocytotic pathway into the cytoplasm and nucleus can be independently corroborated by quantitative fluorescence assays, histochemistry and immunocytochemical methods.

### EXAMPLE 9

### Further Evidence for Aminoglycoside-Permissive Cation Channels in Inner Ear Explants

Deafness and nephrotoxicity are serious consequences of aminoglycoside (AG) therapy. As discussed above, in cultured kidney cells, the non-endocytotic component of fluorescently-labeled gentamicin (GTTR) uptake was regulated by TRPV1 channel agonists and antagonists (pH, Ca⁺⁺, RTX). This example discusses the influence of these modulators on GTTR uptake in explants of bullfrog and murine inner ears.

After live incubation with GTTR, washed, fixed and delipidated inner ear explants revealed extensive cytoplasmic and intra-nuclear GTTR labeling. GTTR uptake was little reduced at 4° C., demonstrating that GTTR uptake was not dependent on endocytosis. Potassium depolarization of explants decreased GTTR uptake. Reduced pH (pH 5) increased GTTR uptake. The specific, competitive, TRPV1 agonist RTX, enhanced GTTR uptake at 0.16 mM Ca⁺⁺, but reduced GTTR uptake at physiological Ca⁺⁺ levels. Low (0.16 mM) Ca⁺⁺ had greater GTTR uptake compared to 0.0 mM Ca⁺⁺.

In TRPV1 −/− cochlear explants, GTTR uptake was clearly altered, being observably higher or lower than wildtype explants, further suggesting that TRPV1 channels are involved in regulating GTTR uptake mechanisms. Regulation of GTTR uptake by specific agonists and antagonists of TRPV 1, in combination with competitive inhibitors (Ca⁺⁺), provides further insight for developing new therapeutic mechanisms to prevent aminoglycoside toxicity.

### EXAMPLE 10

### Cytoplasmic and Intra-Nuclear Binding of Gentamicin does not Require Endocytosis

Materials and Methods

Conjugation: The conjugation of gentamicin to Texas Red (TR) succinimidyl esters (Molecular Probes, OR) was optimized with regard to time, temperature, pH, and ligand/reactive fluorophore ratio to maximize labeling efficiency and to minimize the possibility of over-labeling the gentamicin. This ensures the maintenance of the polycationic nature of gentamicin, which undoubtedly is required for its biological activity. After conjugation, the reaction mixture was separated by reversed phase chromatography using C-18 columns (Burdick and Jackson, Muskegon, Mich.) to isolate the conjugate and thereby eliminate competition from unlabeled gentamicin (GT), or potential contamination by unreacted TR. The isolated gentamicin-Texas Red conjugate (GTTR) was then aliquoted, dried, and stored dessicated, in the dark at −20° C. until required.

Cell Culture: Canine kidney distal tubule (MDCK) cells were a gift from Dr. David Ellison (OHSU). Opossum proximal tubule-derived kidney cells (OK) were purchased from American Type Culture Collection (Manassas, Va.). OKs and MDCKs were cultured in antibiotic-free minimal essential medium (MEM- [symbol] , Invitrogen, Carlsbad, Calif.) supplemented with 10% fetal bovine serum (FBS) at 37° C. with 5% CO₂, 95% air. Complete medium for OK cells was also supplemented with insulin-transferrin-selenium (ITS) and interferon-γ (5 ng/mL). Plates used for OK cells were coated with 0.2% gelatin (in water) for 2 or more hours at 37° C. After draining, plates were treated at room temperature with a 0.9% saline solution containing 10% FBS and 10% rat-tail collagen (gift of Rosemarie Drake-Baumann, PhD, VA Medical Center, Portland, Oreg.), and dried under sterile conditions. Plates were rinsed with complete medium just prior to use. For experimental specimens, cells were seeded into Nunc eight-well coverglass chambers (ISC BioExpress, Kaysville, Utah) in complete medium, and after 3-5 days, both cell types were subconfluent, and MDCK cells had become columnar.

GTTR treatment: Subconfluent MDCK or OK cells were treated with 1 μg/mL of GTTR, in complete medium, for 2 hours at 37° C. or on ice. (The amount of GTTR is expressed as the weight of the gentamicin moiety within the conjugate.) In competition experiments, MDCK cells were simultaneously treated with unlabeled gentamicin (up to 4 mg/mL) for 2 hours.

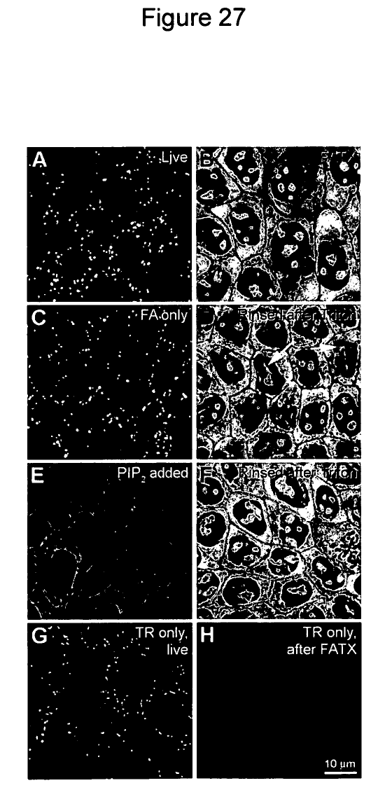

Fixation, delipidation, and washing: After treatment, cells were washed three times with complete medium, and then immediately imaged live (see below; FIG. 27A), or fixed. Most fixation was done by treating cells with 4% formaldehyde and 0.5% Triton X-100 (FATX) in PBS for 45 minutes at room temperature, and followed by extensive washing with PBS (4-6 times, or until foaming in suction pipette ceased) prior to imaging (FIG. 27B). Alternatively, cells were fixed in 4% formaldehyde alone (FA), washed and imaged (FIG. 27C), prior to delipidated with 0.5% Triton X-100 in PBS, washed thoroughly, and imaged again (FIG. 27D). Control cells were incubated with hydrolyzed TR (at the same concentration as the TR moiety in the GTTR experiments) and then imaged live, or fixed, delipidated, washed and then imaged.

PIP₂: Monolayer MDCK cells were grown in coverglass chambers as described above. After fixation, delipidation, washing and imaging (FIG. 27B), cells were treated with 1 mg/mL phosphatidylinositol-4,5-bisphosphate (PIP₂, Echelon Biosciences, Salt Lake City, Utah) for 1.5 hours and re-imaged (FIG. 27E), prior to a second delipidation with 0.5% Triton, washing and imaging again (FIG. 27F).

Spectrophotometry: 3-d scanning fluorescence spectroscopy of solutions containing TR or GTTR with or without (PIP₂) were performed using a Safire fluorescence microplate reader (Tecan, Research Triangle Park, N.C.). GTTR (100 μg/mL; weight as gentamicin in molecule) and PIP₂ (155 μg/mL; approximately equimolar) were mixed vigorously in PBS solution and allowed to stand at room temperature for ½ hour prior to scanning. This was compared to the same concentration of GTTR alone in solution. Similar hydrolyzed Texas Red solutions (at the same concentration as the Texas Red moiety in GTTR solutions), with or without PIP₂, were used as controls. Excitation and emission spectra were obtained over an excitation range of 570-604 nm (bandwidth 5 nm) and an emission range of 610-650 nm (bandwidth 5 nm).

Double-labeling with GTTR and Syto RNASelect. MDCK cells were grown on 8 well chambered coverslips to 40-50% confluency and incubated with GTTR (10 μg/mL) 2 hours in complete supplemented medium at 37° C., 5% CO₂, 95% air. Cells were rinsed twice with 1×PBS, and fixed with ice-cold methanol only for 10 minutes on ice. Subsequently, cells were washed with PBS and incubated with 0.5 μM Syto RNASelect (Molecular Probes, Eugene, Oreg.) for 20 minutes, rinsed and observed using confocal microscopy.

Immunocytochemistry: MDCK cells were grown on 8 well chambered coverslips to 40-50% confluency and incubated with GTTR (5 μg/mL) or unlabeled gentamicin (300 μg/mL) for 2 hours in complete medium, at 37° C. or on ice. Cells were rinsed twice with PBS, fixed with 4% FA, rinsed 3 times with PBS, then permeabilized with ice-cold methanol for 5 minutes, and rinsed 3 times with PBS, as described previously (Steyger et al., 2003). Cells were immunoblocked in 10% goat serum in PBS for 30 minutes, and then incubated with 50 μg/mL rabbit anti-gentamicin IgG (American Quaalex, San Clemente, Calif.) for 1 hour. After washing with 1% goat serum in PBS, cells were further incubated with 20 μg/mL Alexa-488-conjugated goat-anti-rabbit IgG antisera (Molecular Probes, Eugene, Oreg.) for 45 minutes, washed, post-fixed with 4% FA for 15 minutes, and washed again. For immunocytochemical controls, untreated cells not exposed to GTTR or unlabeled GT were fixed, permeabilized and immunoprocessed as for GT- or GTTR-loaded cells. All wells were imaged using confocal microscopy.

Confocal Microscopy: Specimens were observed using a ×60 lens (N.A. 1.4), on a Nikon TE 300 inverted microscope (Melville, N.Y.). Confocal images were collected on a Bio-Rad (Hercules, Calif.) MRC 1024 ES scanning laser system fitted with standard excitation and emission filters for Alexa-488/Syto RNASelect (excitation: 488±12 nm; emission: 515±10 nm) and Texas Red fluorophores (excitation: 568±32 nm; emission: 620±16 nm). Bio-Rad *.pic files acquired using Lasersharp 2000 software exported as *.tif files and prepared for publication using Adobe Photoshop (v.7).

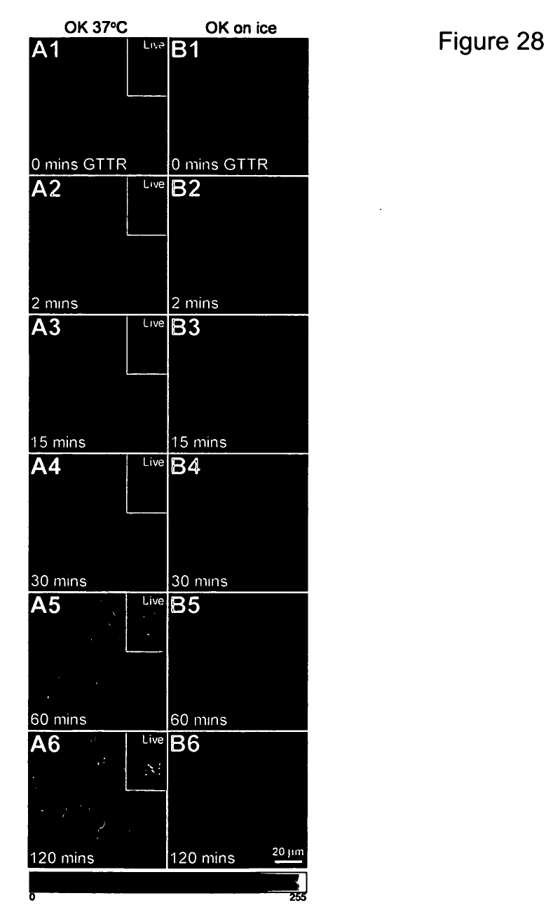

Semi-quantification: Each type of experiment was done multiple times to confirm trends. True quantification of optical sections from cultured cell layers is subject to intensity range variations between experiments, as well within individual images. For this reason, representative images were chosen, and all comparative images were chosen from a single experiment. Intensity differences can often be best illustrated with a color lookup table, hot.lut, and a scale of 0-255 is shown in an intensity bar in FIG. 28.

Results

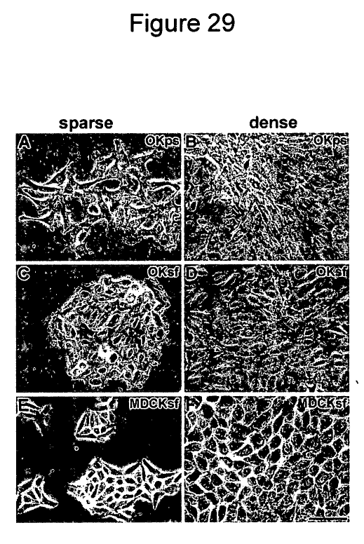

Two cell types were used in these studies, an opossum kidney proximal tubule (OK) clone and a canine kidney distal tubule (MDCK) clone. We chose the OK proximal tubule cell line because of the known clinical toxicity of aminoglycosides in the kidney proximal tubule (Fabrizii et al., 1997; Morin et al., 1984). Although far less subject to aminoglycoside-induced cell death, the MDCK distal tubule cell line was used because the distal tubule is subject to numerous acute effects (Kang et al., 2000; Kidwell et al., 1994; Quamme, 1986). Both were cloned from cultures that had been maintained for extended periods in the absence of the aminoglycoside streptomycin, a common bacterial prophylactic component of many culture media. This was done to optimize the response of cells to the aminoglycoside gentamicin. Although no morphological changes were observed in MDCK cells cultured without streptomycin, OK cells became morphologically and consistently more epitheloid after sustained culture (>7 weeks) in antibiotic-free media (FIG. 29).

Intracellular Distribution of GTTR Uptake

In live MDCK cells, treated with 1 μg/mL purified GTTR for 2 hours in complete medium at 37° C., we found GTTR fluorescence in a punctate, endosome-like distribution in live cells (FIG. 27A), similar to previous studies using Texas Red-labeled gentamicin (Sandoval et al., 1998; Sandoval et al., 2000; Sandoval et al., 2002). Cells fixed with 4% formaldehyde (FA) alone and imaged after washing with PBS exhibited the same vesicular labeling pattern (FIG. 27C).

However, when cells were fixed with 4% FA containing 0.5% Triton X-100 (FATX), no intracellular puncta of fluorescence were observed. Instead, GTTR was observed within the cytoplasm and at distinct intra-nuclear sites (FIG. 27B). In these fixed, delipidated cells, the cytoplasmic labeling reveals little substructure, but the intra-nuclear binding pattern was complex, showing round or ovoid structures, and also tubuloid structures traversing the nucleus (FIG. 27D). When FA-only fixed cells (FIG. 27C) were subsequently treated with 0.5% Triton X-100 (in PBS) alone, and washed with PBS, the punctate, endosome-like, fluorescence had disappeared and the cytoplasmic/nuclear fluorescence was visible (FIG. 27D). This suggests that cytoplasmic/nuclear GTTR was present, but not visible in live cells, or in cells fixed with FA only. It also suggests that penetration of GTTR into the cytoplasm was not an artifact of Triton X-100 being present during the fixation process.

Lipid Quenching of GTTR Fluorescence

When we observed fixed cells in FATX, or in Triton X-100 alone, GTTR fluorescence was significantly reduced. Since Triton X-100, a surfactant used to remove cellular lipids, contains a lipid backbone, we reasoned that cellular lipids might be quenching GTTR fluorescence in live and FA-only fixed kidney cells. There are numerous reports of structural and functional associations of the polycationic gentamicin with cellular anionic phospholipids, and in particular with phosphatidylinositol-4,5-bisphosphate (PIP₂) (Schacht, 1979; Williams et al., 1987). To determine whether such an association might explain the lack of cytoplasmic/nuclear fluorescence in living or FA-only fixed cells, we attempted to quench GTTR fluorescence with PIP₂. Cells fixed with FATX and then rinsed (as in FIG. 29B) were treated with 1 mg/mL PIP₂ and imaged while still in the PIP₂ solution (FIG. 27E). PIP₂ clearly quenched the GTTR fluorescence. This was not due to removal of the GTTR, because when the PIP₂-treated cells were again delipidated with 0.5% Triton X-100 and washed, the GTTR fluorescence regained its former brightness and distribution (FIG. 27F).

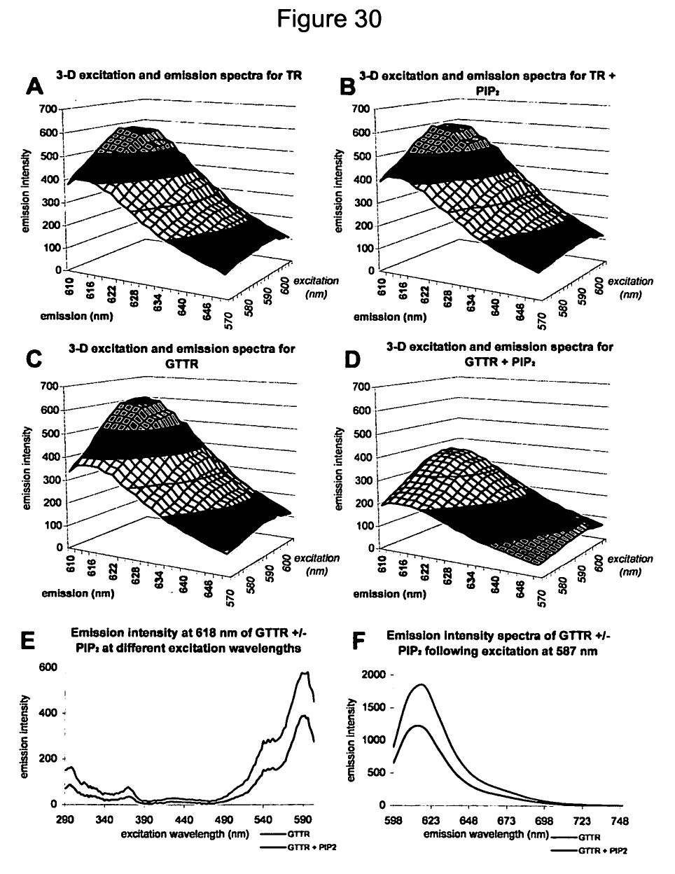

Reduction of GTTR fluorescence by PIP₂ was due to fluorescence quenching and not by excitation or emission spectral shifts. This was ascertained using 3-d scanning fluorescence spectroscopy of solutions containing TR or GTTR (100 μg/mL) with or without PIP₂ (155 μg/mL). FIG. 30 (A-D) shows 3-d scans over an excitation range of 570-604 nm (bandwidth 5 nm) and an emission range of 610-650 nm (bandwidth 5 nm). No quenching was observed when PIP₂ was combined with TR in solution (FIG. 30B) compared with TR alone (FIG. 30A). PIP₂ in solution with GTTR exerted a quenching effect at all wavelengths (FIG. 30D) compared with GTTR alone (FIG. 29C). Using 2-d excitation scanning over a range of 290-604 with emission detected at 618 nm (FIG. 2E) GTTR fluorescence was quenched in the presence of PIP₂. Using 2-d emission scanning over a range of 589-748 nm with excitation at 587 nm, GTTR emission was again quenched at all wavelengths in the presence of PIP₂. The quenching of GTTR (but not TR) fluorescence was probably due to binding of the amphipathic polyanion PIP₂ to the amphipathic polycation gentamicin, and alteration of electrons available to the fluorophore.

Label Specificity:

MDCK cells treated with the same dose of hydrolyzed TR (based on fluorescence equivalent to GTTR in solution) exhibited fluorescently-labeled vesicles when imaged live (FIG. 27G), but not after FATX treatment (FIG. 27H). Thus, the gentamicin moiety of the GTTR conjugate is required for accumulation in the cytoplasmic/intra-nuclear compartment, but not in the endosomal compartment.

Intra-Nuclear Labeling

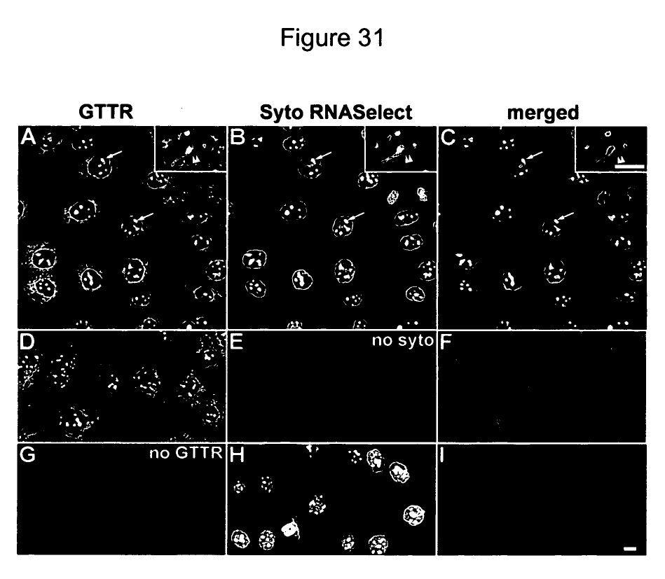

To determine if intra-nuclear GTTR labeling was co-localized with nuclear RNA, GTTR-loaded MDCK cells were fixed with methanol only, and labeled with Syto RNASelect. The globular intra-nuclear structures labeled by GTTR (FIG. 31A) were intensely co-labeled by Syto RNASelect (FIG. 31B,C), and are presumed to be nucleoli (Haugland et al., 2004). The trans-nuclear tubular structures were also co-labeled with both GTTR and Syto RNASelect (FIG. 31 insets). Unlabeled gentamicin (2 mg/mL) did not interfere with the binding of Syto RNASelect to nuclear material (data not shown). Cells incubated with GTTR (FIG. 31D) without subsequent Syto RNASelect treatment had negligible fluorescence at the 515 nm emission wavelength (FIG. 31E). Untreated cells fixed with methanol and labeled with fluorescent Syto RNASelect (515 nm emission; FIG. 4H) did not exhibit fluorescence bleed-through into the red (GTTR) channel (620 nm; FIG. 31G).

Characteristics of Cytoplasmic and Nuclear Binding: Saturability, Time and Temperature Effects

Saturability in the binding of a ligand demonstrates the existence of a limited number of binding sites and is the hallmark of specificity. Saturability is demonstrated if binding of a labeled ligand can be serially reduced by increasing quantities of the native, unlabeled ligand. Such data also demonstrate that the labeled ligand remains sufficiently bio-relevant that its distribution is a valid report of the distribution of the unlabeled molecule. Biological processes are time- and temperature-dependent, for example, crossing a barrier such as the plasma membrane. In particular, at low temperatures (cells held over ice) endosomal traffic would be halted, but permeation through pores or channels could continue, albeit more slowly.

Compartment Saturability

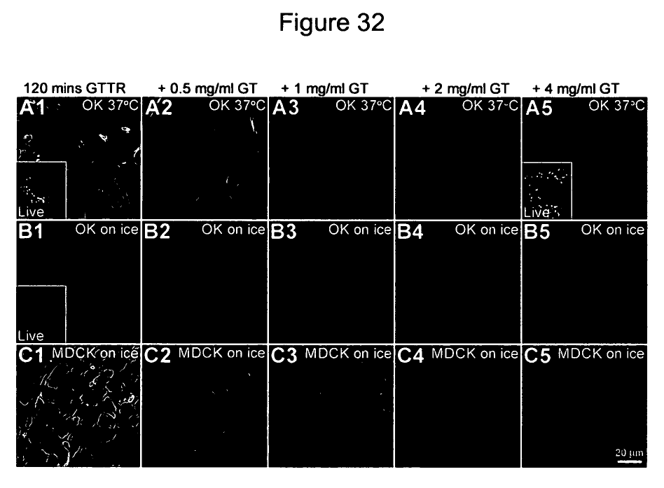

Both OK and MDCK cells were treated with GTTR at 1 μg/mL in complete culture medium for 2 hours at either 37° C. or over ice. These cells were also treated with a dose range of 0 to 4000 μg/mL of unlabeled GT. Cells were washed and imaged live (as in FIG. 27A), then fixed with FATX, and washed again with PBS prior to re-imaging (as in FIG. 27B). In live cells at 37° C., there was a large accumulation of GTTR-labeled puncta and this accumulation of endosome-like puncta was not visibly altered by even the highest doses of unlabeled GT (FIG. 32, A5, inset). After FATX fixation and wash, cytoplasmic and nuclear fluorescence was observed in both cell types and at both temperatures (FIG. 32, A1, B1, and C1). Fluorescence was reduced in cells treated on ice, but, notably, still occurred (FIG. 32, B1, C1). At both temperatures and in both cell lines, increasing doses of unlabeled GT serially reduced the amount of GTTR observed in both the cytoplasm and nucleoli (FIG. 32, A2-5, B2-5, and C2-5). Thus, cytoplasmic, but not endosomal, uptake of GTTR was saturable. Cells treated on ice and imaged live exhibited no endosome-like fluorescent puncta (FIG. 32, B1 inset). These results support two conclusions. Firstly, the cytoplasmic/nuclear compartment, but not the endosomal compartment, exhibited the characteristic of saturability that demonstrates specificity. This argues against the endosomal compartment being the source of the GTTR bound to the cytoplasmic/nuclear sites, either as a biological transit component or as a source for (artifactual) translocation during fixation. In addition, the saturable cytoplasmic uptake of GTTR by cells treated on ice demonstrates that gentamicin entry into the cytoplasmic compartment does not require endocytosis.

Time and Temperature

OK cells were treated with 1 μg/mL of GTTR in complete culture medium, at 37° C. or on ice, for increasing time periods. Binding of GTTR within the cytoplasm and nucleus increased over time both at 37° C. (FIG. 28 A1-6) and (more slowly) on ice (FIG. 28, B1-6). At 37° C., cytoplasmic binding occurred prior to visible uptake into endosomes (FIG. 28, compare A1-6 with insets, particular A2, and A3), consistent with FIG. 5 showing that cytoplasmic uptake of GTTR does not require endocytosis. (Note that live images were acquired without washing out GTTR from the extracellular medium, so fluorescence is visible outside the cells.) No endosomes were observed on OK cells treated on ice for 2 hours (FIG. 28, B1 inset). GTTR was also taken up by MDCK cells as a function of time (data not shown). Increased binding over time at both temperatures reinforces the premise that cytoplasmic uptake of GTTR is a biological phenomenon and occurs in the absence of endocytosis.

Corroboration of GTTR Distribution by Immunocytochemistry

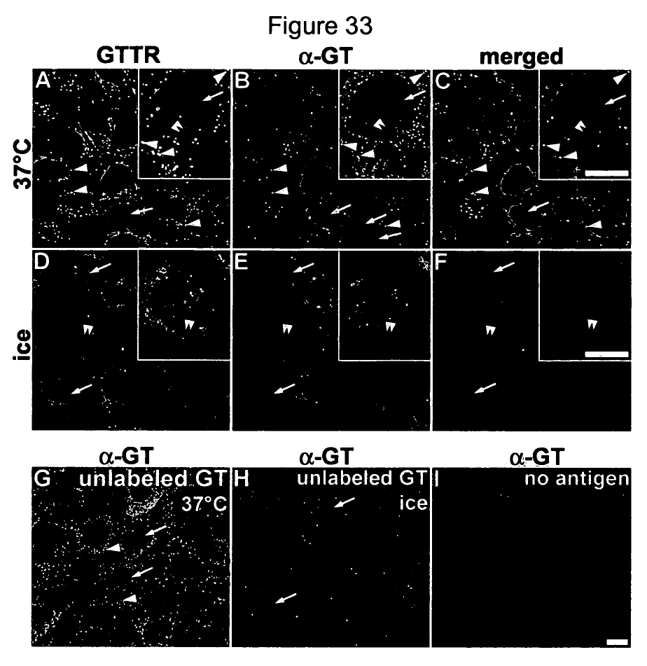

MDCK cells were loaded with GTTR or unlabeled gentamicin at 37° C.; or on ice, for two hours, then fixed with FA only, permeabilized with methanol and immunolabeled with gentamicin antisera. At 37° C., GTTR fluorescence was observed throughout the cytoplasm, and as endosome-like puncta. In the nucleus, GTTR labeled the nucleoli and trans-nuclear tubules (FIG. 33A). Immunolabeling of GTTR with gentamicin antisera revealed close correlation with GTTR fluorescence, including widespread diffuse cytoplasmic immunolabeling, and immunolabeling of GTTR-loaded vesicles (FIG. 33 B,C). GTTR-labeled trans-nuclear tubules (FIG. 33A, inset) were also immunolabeled by gentamicin antisera (FIG. 33B, inset).

In cells loaded with GTTR on ice, extensive diffuse cytoplasmic GTTR fluorescence was observed, together with labeled trans-nuclear tubules (FIG. 33D and inset) that were also immunolabeled by gentamicin antisera (FIG. 33E). Endosome-like puncta of GTTR or immunolabeled GT fluorescence, observed in cells treated at 37° C., were absent in cells treated on ice (compare FIG. 33A with 33D, and FIG. 33B with 33E). Gentamicin antisera did not label GTTR-fluorescing nucleoli (FIG. 33B,E).

When MDCK cells were loaded with unlabeled gentamicin at 37° C. (FIG. 33G) or on ice (FIG. 33H), diffuse immunofluorescence was observed in the cytoplasm and nucleoplasm, but excluded from nucleoli (as in immunolabeled GTTR specimens, FIG. 7B,E), even at the higher gentamicin doses used here (300 μg/mL). Cells incubated with 5 μg/mL unlabeled gentamicin for 2 hours revealed similar but much weaker patterns of immunolabeling (data not shown). The distribution of immunolabeled GTTR and GT closely correlated with each other, and with the distribution of GTTR fluorescence (except for the nucleoli). Gentamicin immunofluorescence was not replicated by immunoprocessing of GT- or GTTR-loaded cells with antigen-adsorbed primary antibodies (data not shown); or in untreated cells (i.e., no GTTR or GT loading), fixed and immunoprocessed with primary and secondary antisera (FIG. 33I).

Discussion

GTTR as an Imaging Probe

The biological relevance of GTTR was demonstrated by showing in FIG. 32 that the fluorescent probe could be competed off its binding sites by native gentamicin. In this example, purified GTTR is used as a probe to exhibit and validate a novel intracellular gentamicin distribution pattern. We examined both spatial distribution and intensity differences of GTTR in two kidney cell lines. The use of fluorescent ligands and confocal imaging offers considerable information regarding distribution of ligands in fixed specimens (neither sectioned nor fractionated). Instructive and reproducible differences in fluorescence intensity can be observed within an image or between images subjected to different experimental conditions. For fluorescent microscopy images of biological specimens subject to natural variation among cells, especially at high resolution, numerical intensity comparisons are difficult to validate. It is more instructive to be able to see “clearly more”, “clearly less”, or “fairly similar” fluorescence for quantitative comparisons. Intensity differences as the result of experimental conditions are visually clear, and provide the phenomenological information for the conclusions of this study. For these reasons, the images do not include numbers, and intensity differences have not been graphed.

Cytoplasmic Penetration

This example describes a more rapid uptake of fluorescently-labeled gentamicin throughout the cytoplasm and at intra-nuclear sites than previously described (Sandoval et al., 1998; Sandoval et al., 2004; Sandoval et al., 2000). Finding gentamicin in the cytoplasm is consistent with earlier studies using radiolabeling or biochemical extraction (Ding et al., 1995; Ding et al., 1997; Wedeen et al., 1983). Recent fluorescence and immunocytochemical studies in fixed, methanol-permeabilized frog saccular explants also showed gentamicin localization in vesicles, in the cytoplasm and the nucleus (Steyger et al., 2003). The cytoplasmic distribution of gentamicin is also consistent with clinical studies in which gentamicin was able to suppress premature stop codons in genetic diseases (Clancy et al., 2001; Clemens et al., 2001; Keeling et al., 2002; Schulz et al., 2002). However, penetration of gentamicin directly into the cytoplasm in the absence of endocytosis (i.e. cells held over ice) is contrary to recent reports describing gentamicin-Texas Red uptake by endocytosis, and subsequent release into the cytoplasm from vesicular compartments (Sandoval et al., 2004).

Cytoplasmic and nuclear GTTR fluorescence could not be seen in live cells in our studies (FIG. 29, B1 and B2) or in previous reports (Dunn et al., 2003), but only after both fixation and detergent delipidation as described here, and elsewhere (Sandoval et al., 2004). Probably the most important difference between our studies and earlier reports using fluorescently labeled gentamicin is the degree of “permeabilization” used (Sandoval et al., 1998; Sandoval et al., 2004; Sandoval et al., 2000; Sandoval et al., 2002). In those studies, Triton X-100 was used at a concentration of 0.05-0.1% for 10 minutes. In our studies, a 0.5% concentration was used for at least 30 minutes, and then thoroughly rinsed, thus more effectively removing cellular lipids. In addition, Sandoval et al. (1998, 2000, 2004) used much higher doses of fluorescent gentamicin, often by two-three orders of magnitude.

In earlier studies using the kidney cell line LLC-PK1, Sandoval et al. (1998) showed vesicular uptake of Texas Red-labeled gentamicin, but not of unconjugated Texas Red. In our studies, the same dose of hydrolyzed TR (as that in GTTR) resulted in fluorescently-labeled vesicles in live cells (FIG. 27G), which was not seen after FATX treatment (FIG. 27H). This suggests non-specific endocytotic uptake of hydrolyzed TR (and by implication GTTR). However, since hydrolyzed TR did not label the cytoplasmic and nuclear domains, the gentamicin moiety of the GTTR conjugate was required for accumulation in those domains. This was further demonstrated by cold inhibition of endocytosis, during which cytoplasmic uptake of gentamicin still occurred (FIGS. 32 and 33), as with FM1-43 in sensory hair cells, implicating ion channels as a mechanism of fluorophore uptake (Meyers et al., 2003).

Fluorescence Quenching

Triton X-100 and PIP₂ reduced the fluorescence of GTTR. The Texas Red molecule is known to exhibit little change in fluorescence emission in response to environmental conditions, such as changes in pH (Haugland et al., 1996), although its fluorescence can be self-quenched at high concentrations. We also find no reports of environmental sensitivity when Texas Red is conjugated to large molecules, such as antibodies (Haugland et al., 1996; Haugland et al., 2004). But, gentamicin, a mixture of 3 isoforms with an average MW of 469, is a polyamine, with 2 or 3 amine side groups remaining after conjugation with Texas Red. Deprotonation of these amines alters the fluorescent emission, measured by fluorimetry, of Texas Red covalently attached to the gentamicin, compared to unconjugated Texas Red in solution (unpublished studies). In both confocal imaging and fluorimetry, however, both excitation and emission wavelengths are selected with band-pass filters, so do not distinguish between (apparent) fluorescence quenching and an environmentally-induced spectral shift in the excitation or emission spectrum, or both. Such shifts could produce peaks that would miss the band pass filters and appear as quenching even if emission were enhanced at a different wavelength. However, spectral scans of GTTR in solution with or without PIP₂ over an excitation range of 290-609 nm and emission range of 598-750 nm produced 2- or 3-dimensional fluorescence maps which showed clearly that PIP₂ attenuated GTTR fluorescence at all wavelengths (FIG. 30). PIP₂ had no effect on Texas Red alone in solution, indicating that PIP₂ was interacting with the gentamicin moiety of GTTR. Yet, in those experiments, and in FIG. 27 A3, PIP₂ did not completely block GTTR fluorescence, although almost no GTTR fluorescence was observed in live cells treated at temperatures incompatible with endosomal uptake. In solution, much higher concentrations of PIP₂ might have completely blocked fluorescence. In vivo, other lipids (e.g., phosphidylserines etc) or cellular quenching mechanisms may be involved. Additionally, PIP₂ may not bind as effectively to intracellular GTTR that had been cross-linked, via one or more of its amine groups as it can with free GTTR, which has 2 or 3 pendant amine groups in solution.

Intra-Nuclear Labeling

Gentamicin is known to bind to the major groove of prokaryotic and eukaryotic ribosomal RNA, a major component of nucleoli and ribosomes (Lynch et al., 2001; Yoshizawa et al., 1998). GTTR was co-localized with the RNA-specific Syto RNASelect fluorophore in intra-nuclear structures, identified as nucleoli (Haugland et al., 2004). In addition, the trans-nuclear tubular structures were also co-labeled with both Syto RNASelect and GTTR, suggesting that these sites were also rich in RNA as well as other gentamicin binding sites.

Characteristics of GTTR Accumulation into the Cytoplasmic Compartment

With either the proximal, OK, or distal, MDCK, kidney cell lines, binding of GTTR was time and temperature dependent, and saturable at both cytoplasmic and intra-nuclear sites. GTTR binding serially decreased as a function of increasing concentrations of unlabeled gentamicin in the culture media. Competition with the native molecule shows that intracellular gentamicin binding sites are limited in number, and that the labeled molecule retains the biological characteristics of the native molecule, at least with regard to uptake and distribution (GTTR is a tracer, and would not be used to study toxicity or other physiological activities). This demonstrated the biological specificity of GTTR binding at these sites. At 37° C., we also observed vesicular uptake of GTTR over time. Unlike one previous, unconfirmed, report (Sandoval et al., 1998), we were unable, in numerous experiments, to see any significant reduction in vesicular uptake of GTTR, even using high excesses of native gentamicin (>4000×). A possible explanation for this difference is that extended gentamicin treatment (1 mg/mL) of a porcine kidney tubule cell line (LLC PK1) inhibits endocytosis (Kempson et al., 1989). Thus the 12 mg/mL dose of gentamicin used in earlier studies LLC PK1 cells (Sandoval et al., 1998) could have reduced endocytotic uptake of labeled gentamicin in a non-competitive manner. At 4 mg/mL gentamicin in OK and MDCK cells, for 2 hours, we observed only flattening of cells, which slightly altered the apparent distribution of vesicles, so a small decrease in vesicle number would be difficult to observe or document. But, vesicular uptake of GTTR was little changed, unlike the near extinction of cytoplasmic and nuclear binding of GTTR at the highest doses of unlabeled gentamicin.

This suggests that a large fraction of the vesicular uptake is associated with non-specific, fluid-phase endocytosis, since it is not saturable, confirming a previous report (Decorti et al., 1999). This non-specific endocytotic uptake of aminoglycosides is also consistent with the observation that GTTR within the vesicular compartment is only weakly associated with to endosomal (or membranous) components during aldehyde fixation and is ultimately washed away during detergent permeabilization (see FIG. 27). Furthermore, hydrolyzed Texas Red alone was seen only in vesicles of live cells, but it was not seen in the cytoplasmic or nuclear compartments after FATX treatment (FIG. 27).

Endocytosis of GTTR was not visibly reduced at a 4000-fold excess of competing unlabeled gentamicin, yet GTTR fluorescence in the cytoplasmic and nuclear compartments was greatly reduced. Cytoplasmic GTTR fluorescence was also observed in cells treated on ice to inhibit endocytosis. Inhibition of endocytosis in cells treated on ice was verified by live imaging of GTTR-loaded cells incubated on ice, and by the absence of endosome-like fluorescent puncta of GTTR or immunolabeling. In cells treated on ice, increasing concentrations of unlabeled gentamicin also serially reduced cytoplasmic and nuclear GTTR fluorescence. Taken together, these data strongly negate the possibility that GTTR released from endosomes during FATX treatment are the source of cytoplasmic and nuclear GTTR fluorescence.

Corroboration of GTTR Distribution by Immunocytochemistry

At 37° C., after FA fixation and methanol treatment, GTTR-loaded cells displayed both diffuse and punctate (vesicular) cytoplasmic fluorescence for both GTTR and immunolabeled GTTR, Gentamicin antibodies also co-localized with GTTR-labeled trans-nuclear tubules. In cells loaded with GTTR on ice, no punctate GTTR or immunolabeling could be observed, however, diffuse cytoplasmic and nucleoplasmic GTTR and immunofluorescence were both visible. GTTR-labeled nucleoli were negligibly immunolabeled. Immunocytochemical detection of unconjugated gentamicin loaded into cells on ice (and at 37° C.) also correlated with the diffuse cytoplasmic distribution of both GTTR and immunolabeled GTTR. Thus, immunodetection of GT or GTTR display similar distributions pattern as GTTR (except for nucleoli), demonstrating that fluorescence of GTTR was due to the gentamicin-Texas Red conjugate, and not due to cleaved TR molecules. The specific, high affinity binding of the gentamicin molecule (or moiety) to RNA may lead to steric hindrance or immunogenic site masking and account for the negligible immunolabeling of nucleoli by gentamicin antisera. Indeed, GTTR labeling of the nucleoli may indicate one advantage of the GTTR conjugate over immunodetection of gentamicin because it eliminates the potential masking of immunogenic sites when gentamicin is specifically bound to intracellular ligands (e.g., RNA).

GTTR does not fluoresce within the cytoplasm of live cells. This illustrates an additional pitfall when attaching fluorophores to small molecules. The chemical nature of the molecule itself can influence the fluorescence of the attached fluorophore. In the case of GTTR, the electron densities of the basic amine groups on the gentamicin molecule were apparently modified while interacting with the acidic phospholipids (or other charged molecules) within the cytoplasm of live cells in a manner that reduced the fluorescence efficiency of the Texas Red moiety. This effect is undoubtedly largely responsible for the difference between our findings and other studies using fluorescence imaging of GTTR. In this example, we have shown biorelevant, non-endocytotic uptake of gentamicin, with cytoplasmic and intra-nuclear binding sites.

### References for Example 10

Aran, J. M., Dulon, D., Hiel, H., Erre, J. P., Aurousseau, C. 1993. [Ototoxicity of aminosides: recent results on uptake and clearance of gentamycin by sensory cells of the cochlea]. Rev Laryngol 0 to 1 Rhinol 114, 125-8.
Beauchamp, D., Gourde, P., Bergeron, M. G. 1991. Subcellular distribution of gentamicin in proximal tubular cells, determined by immunogold labeling. Antimicrob Agents Chemother 35, 2173-9.
Begg, E. J., Barclay, M. L. 1995. Aminoglycosides—50 years on. Br J Clin Pharmacol 39, 597-603.
Belus, A., White, E. 2001. Effects of antibiotics on the contractility and Ca2+ transients of rat cardiac myocytes. Eur J Pharmacol 412, 121-6.
Clancy, J. P., Bebok, Z., Ruiz, F., King, C., Jones, J., Walker, L., Greer, H., Hong, J., Wing, L., Macaluso, M., Lyrene, R., Sorscher, E. J., Bedwell, D. M. 2001. Evidence that systemic gentamicin suppresses premature stop mutations in patients with cystic fibrosis. Am J Respir Crit Care Med 163, 1683-92.
Clemens, P. R., Duncan, F. J. 2001. Progress in gene therapy for Duchenne muscular dystrophy. Curr Neurol Neurosci Rep 1, 89-96.
de Jager, P., van Altena, R. 2002. Hearing loss and nephrotoxicity in long-term aminoglycoside treatment in patients with tuberculosis. Int J Tuberc Lung Dis 6, 622-7.
De la Chapelle-Groz, B., Athias, P. 1988. Gentamicin causes the fast depression of action potential and contraction in cultured cardiocytes. Eur J Pharmacol 152, 111-20.
Decorti, G., Malusa, N., Furlan, G., Candussio, L., Klugmann, F. B. 1999. Endocytosis of gentamicin in a proximal tubular renal cell line. Life Sci 65, 1115-24.
Ding, D., Jin, X., Zhao, J. 1995. [Accumulation sites of kanamycin in cochlear basal membrane cells]. Zhonghua Er Bi Yan Hou Ke Za Zhi 30, 323-5.
Ding, D., Jin, X., Zhao, J. 1997. [Accumulation sites of kanamycin in the organ of Corti by microautoradiography]. Zhonghua Er Bi Yan Hou Ke Za Zhi 32, 348-9.
Dunn, K. W., Sandoval, R. M., Molitoris, B. A. 2003. Intravital imaging of the kidney using multiparameter multiphoton microscopy. Nephron Exp Nephrol 94, e7-11.
Fabrizii, V., Thalhammer, F., Horl, W. H. 1997. [Aminoglycoside-induced nephrotoxicity]. Wien Klin Wochenschr 109, 830-5.
Fiekers, J. F. 1983. Effects of the aminoglycoside antibiotics, streptomycin and neomycin, on neuromuscular transmission. II. Postsynaptic considerations. J Pharmacol Exp Ther 225, 496-502.
Gilbert, D. N., Wood, C. A., Kohlhepp, S. J., Kohnen, P. W., Houghton, D. C., Finkbeiner, H. C., Lindsley, J., Bennett, W. M. 1989. Polyaspartic acid prevents experimental aminoglycoside nephrotoxicity. J Infect Dis 159, 945-53.
Goldhill, J. M., Rose, K., Percy, W. H. 1996. Effects of antibiotics on epithelial ion transport in the rabbit distal colon in-vitro. J Pharm Pharmacol 48, 651-6.
Hall, J. W., 3rd, Herndon, D. N., Gary, L. B., Winkler, J. B. 1986. Auditory brainstem response in young burn-wound patients treated with ototoxic drugs. Int J Pediatr Otorhinolaryngol 12, 187-203.
Hashino, E., Shero, M., Salvi, R. J. 1997. Lysosomal targeting and accumulation of aminoglycoside antibiotics in sensory hair cells. Brain Res 777, 75-85.
Haugland, R. P., Spence, M. T. Z., Johnson, I. D. 1996. Handbook of fluorescent probes and research chemicals. 6th ed. Molecular Probes, Eugene, Oreg., USA (4849 Pitchford Ave., Eugene 97402).
Haugland, R. P., Gregory, J., Spence, M. T. Z., Johnson, I. D. 2004. Handbook of fluorescent probes and research chemicals (online version). 9th ed. Molecular Probes, Eugene, Oreg., USA (4849 Pitchford Ave., Eugene 97402).
Hiel, H., Schamel, A., Erre, J. P., Hayashida, T., Dulon, D., Aran, J. M. 1992. Cellular and subcellular localization of tritiated gentamicin in the guinea pig cochlea following combined treatment with ethacrynic acid. Hear Res 57, 157-65.
Hock, R., Anderson, R. J. 1995. Prevention of drug-induced nephrotoxicity in the intensive care unit. J Crit Care 10, 33-43.
Imamura, S. I., Adams, J. C. 2003. Distribution of Gentamicin in the Guinea Pig Inner Ear after Local or Systemic Application. J Assoc Res Otolaryngol (in press), First Online JARO web site.
Kahlmeter, G., Dahlager, J. I. 1984. Aminoglycoside toxicity—a review of clinical studies published between 1975 and 1982. J Antimicrob Chemother 13 Suppl A, 9-22.
Kang, H. S., Kerstan, D., Dai, L., Ritchie, G., Quamme, G. A. 2000. Aminoglycosides inhibit hormone-stimulated Mg2+ uptake in mouse distal convoluted tubule cells. Can J Physiol Pharmacol 78, 595-602.
Keeling, K. M., Bedwell, D. M. 2002. Clinically relevant aminoglycosides can suppress disease-associated premature stop mutations in the IDUA and P53 cDNAs in a mammalian translation system. J Mol Med 80, 367-76.
Kempson, S. A., Ying, A. L., McAteer, J. A., Murer, H. 1989. Endocytosis and Na+/solute cotransport in renal epithelial cells. J Biol Chem 264, 18451-6.
Kidwell, D. T., McKeown, J. W., Grider, J. S., McCombs, G. B., Ott, C. E., Jackson, B. A. 1994. Acute effects of gentamicin on thick ascending limb function in the rat. Eur J Pharmacol 270, 97-103.
Leehey, D. J., Braun, B. I., Tholl, D. A., Chung, L. S., Gross, C. A., Roback, J. A., Lentino, J. R. 1993. Can pharmacokinetic dosing decrease nephrotoxicity associated with aminoglycoside therapy. J Am Soc Nephrol 4, 81-90.
Lynch, S. R., Puglisi, J. D. 2001. Structure of a eukaryotic decoding region A-site RNA. J Mol Biol 306, 1023-35.
Meyers, J. R., MacDonald, R. B., Duggan, A., Lenzi, D., Standaert, D. G., Corwin, J. T., Corey, D. P. 2003. Lighting up the senses: FM1-43 loading of sensory cells through nonselective ion channels. J Neurosci 23, 4054-65.
Miller, J. J. 1985. Handbook of ototoxicity CRC Press, Boca Raton.
Morin, J. P., Fillastre, J. P., Olier, B. 1984. Antibiotic nephrotoxicity. Chemioterapia 3, 33-40.
Myrdal, S. E., Steyger, P. S. 2004. TRPV1 Channel Mediates Gentamicin Entry In Cultured Kidney Cells. ARO Midwinter Meeting Abstracts 27, 135.
Quamme, G. A. 1986. Renal handling of magnesium: drug and hormone interactions. Magnesium 5, 248-72.
Sandoval, R., Leiser, J., Molitoris, B. A. 1998. Aminoglycoside antibiotics traffic to the Golgi complex in LLC-PK1 cells. J Am Soc Nephrol 9, 167-74.
Sandoval, R. M., Molitoris, B. A. 2004. Gentamicin traffics retrograde through the secretory pathway and is released in the cytosol via the endoplasmic reticulum. Am J Physiol Renal Physiol 286, F617-24.
Sandoval, R. M., Dunn, K. W., Molitoris, B. A. 2000. Gentamicin traffics rapidly and directly to the Golgi complex in LLC-PK(1) cells. Am J Physiol Renal Physiol 279, F884-90.
Sandoval, R. M., Bacallao, R. L., Dunn, K. W., Leiser, J. D., Molitoris, B. A. 2002. Nucleotide depletion increases trafficking of gentamicin to the Golgi complex in LLC-PK1 cells. Am J Physiol Renal Physiol 283, F1422-9.
Schacht, J. 1979. Isolation of an aminoglycoside receptor from guinea pig inner ear tissues and kidney. Arch Otorhinolaryngol 224, 129-34.
Schulz, A., Sangkuhl, K., Lennert, T., Wigger, M., Price, D. A., Nuuja, A., Gruters, A., Schultz, G., Schoneberg, T. 2002. Aminoglycoside pretreatment partially restores the function of truncated V(2) vasopressin receptors found in patients with nephrogenic diabetes insipidus. J Clin Endocrinol Metab 87, 5247-57.
Steyger, P. S., Peters, S. L., Rehling, J., Hordichok, A., Dai, C. F. 2003. Uptake of gentamicin by bullfrog saccular hair cells in vitro. J Assoc Res Otolaryngol 4, 565-78.
Tulkens, P. M. 1989. Nephrotoxicity of aminoglycoside antibiotics. Toxicol Lett 46, 107-23.
van Lent-Evers, N. A., Mathot, R. A., Geus, W. P., van Hout, B. A., Vinks, A. A. 1999. Impact of goal-oriented and model-based clinical pharmacokinetic dosing of aminoglycosides on clinical outcome: a cost-effectiveness analysis. Ther Drug Monit 21, 63-73.
Wedeen, R. P., Batuman, V., Cheeks, C., Marquet, E., Sobel, H. 1983. Transport of gentamicin in rat proximal tubule. Lab Invest 48, 212-23.
Williams, S. E., Zenner, H. P., Schacht, J. 1987. Three molecular steps of aminoglycoside ototoxicity demonstrated in outer hair cells. Hear Res 30, 11-8.
Williams, S. E., Smith, D. E., Schacht, J. 1987. Characteristics of gentamicin uptake in the isolated crista ampullaris of the inner ear of the guinea pig. Biochem Pharmacol 36, 89-95.
Yoshizawa, S., Fourmy, D., Puglisi, J. D. 1998. Structural origins of gentamicin antibiotic action. Embo J 17, 6437-48.
Zhou, Y., Zhao, Z. Q. 2002. Effects of neomycin on high-threshold Ca(2+) currents and tetrodotoxin-resistant Na(+) currents in rat dorsal root ganglion neuron. Eur J Pharmacol 450, 29-35.
Zhou, Y., Zhou, Z. S., Zhao, Z. Q. 2001. Neomycin blocks capsaicin-evoked responses in rat dorsal root ganglion neurons. Neurosci Lett 315, 98-102.

### EXAMPLE 11

### TRPV1 Regulators Mediate Gentamicin Penetration of Cultured Kidney Cells

Materials and Methods for this example: All materials from Sigma-Aldrich (St. Louis, Mo.), unless otherwise stated.

Conjugation

The conjugation of gentamicin to Texas Red (TR) succinimidyl esters (Molecular Probes, OR) was done as described in Example 10 isolated by reversed-phase chromatography, then aliquoted, dried, and stored dessicated, dark and at −20° C. until required.

Cell Culture

Canine kidney distal tubule MDCK cells were a gift from Dr. David Ellison (OHSU), but are commercially available (ATCC). Cells were routinely cultured in antibiotic and phenol red-free Dulbecco's minimal essential medium (MEMα, Invitrogen, Ca) with 10% fetal bovine serum (FBS) and kept at 37° C. with 5% CO₂, 95% air. For testing, cells were seeded into 8-well coverglass chambers (ISC BioExpress) at 3000 cells/well and grown for 5 days, when they had become subconfluent, columnar and had time to develop tight junctions.

Experimental Procedures

Cells were washed three times with the buffer to be used in the particular experiment, treated with GTTR and experimental variable for 30 or 60 seconds at 20° C., precluding endocytosis. Following treatment, cells were rinsed three times with buffer, then fixed and delipidated with 4% formaldehyde plus 0.5% Triton X-100 (FATX) for 45 minutes. Following fixation, cells were rinsed with PBS (Invitrogen, CA) for at least 4-6 times, or until foaming in the suction pipette ceased. In contrast to previous experiments, no FBS was present in the treatment media, allowing for more rapid uptake of the antibiotic-based GTTR.

Extracellular Potassium

MDCK cells were washed with Hank's buffered salt solution (HBSS; Invitrogen, CA), then placed into buffers of varying potassium concentrations. HBSS was mixed with equi-osmolar KCl/HBSS to produce the required K⁺ concentrations. Cells were treated with 1 μg/mL GTTR for 1 minute, then washed and fixed as described above.

Extracellular Calcium

Cells were washed with PBS (Invitrogen, CA), then placed into buffers containing varying concentrations of calcium, obtained by mixing HBSS with equimolar CaCl₂, each at pH 7.3. Cells were treated with 5 μg/mL of GTTR for 30 seconds, then washed and fixed as above.

Extracellular pH

Cells were washed with saline, then treated with 5 μg/mL of GTTR in saline buffers of varying pH for 30 seconds, then washed and fixed as above. Sodium hydroxide and hydrochloric acid were used to alter pH. These experiments were performed in three different saline buffers: (i) PBS (no calcium), (ii) a mixture of one part PBS and one part HBSS for a final calcium concentration of 0.32 mM, and (iii) a mixture of three parts PBS to one part HBSS for a final calcium concentration of 0.97 mM.

TRPV1 Agonists and Antagonists

Cells were washed three times with Ca⁺⁺-free saline (0.9% NaCl), then treated with 5 μg/mL GTTR and one of the following for 30 seconds: TRPV1 agonists (resiniferatoxin [RTX]; or anandamide [AND]; Alexis Biochemicals, San Diego, Calif.); or antagonists (iodo-RTX, or SB 366791; Tocris, Ellisville, Mo.). Cells were then washed in saline and fixed as above. (No EGTA was present in the saline to bind residual calcium, as the cells would have detached from the coverglasses during treatment and subsequent washing. Thus there was, undoubtedly, a minor amount of calcium during treatment.)

Non-Specific Cation Channel Antagonists

Cells were washed three times with Ca⁺⁺-free 0.9% NaCl, then treated with 5 μg/mL GTTR and one of the following for 30 seconds: 100 μM Ruthenium Red, 100 μM Ruthenium Red plus one of the TRPV1 agonists or antagonists described above; or lanthanum (La⁺⁺⁺) at 0.5 or 5 mM.

Immunocytochemistry

MDCK cells grown on 8 well chambered coverslips to 30-40% confluency were incubated with unlabeled gentamicin (300 μg/mL) for 30 seconds at 20° C., in the presence of 0, 0.5 or 5 mM La⁺⁺⁺. Cells were rinsed twice with PBS, fixed with 4% FA, then permeabilized with ice-cold methanol for 5 minutes, and rinsed 3 times with PBS, as described previously (Steyger et al., 2003). Cells were immunoblocked in 10% goat serum in PBS for 30 minutes, and then incubated with 50 μg/mL rabbit anti-gentamicin IgG (American Quaalex, San Clemente, Calif.) for 1 hour. After washing with 1% goat serum in PBS, cells were further incubated with 20 μg/mL Alexa-488-conjugated goat-anti-rabbit IgG antisera (Molecular Probes, Eugene, Oreg.) for 45 minutes, washed, post-fixed with 4% FA for 15 minutes, and washed again. For immunocytochemical controls, the primary IgG were omitted, or adsorbed with excess gentamicin (3 mg/mL) for 30 minutes prior to addition to cells. All wells were imaged using confocal microscopy.

Confocal Microscopy

Specimens were observed using a ×60 lens (N.A. 1.4), on a Nikon TE 300 inverted microscope. Confocal images (512×512 pixels) were collected on a Bio-Red 1024 ES scanning laser system using the same confocal settings (laser intensity, iris, gain, offset) for each experiment. Bio-Rad *.pic files were converted to *.tif files, and prepared for publication using Adobe Photoshop (v.7).

Semi-Quantification

Each type of experiment was done multiple times to confirm trends. True quantification of optical section from cultured cell layers is subject to intensity range variations between experiments, as well within individual images. For this reason, representative experiments were chosen (all comparisons were taken from a single experiment) and intensity differences were illustrated with a color lookup table, hot.lut.

Results

MDCK cells were used as a model system to determine whether GTTR uptake would be modulated by conditions known to produce or modify a cation current through the TRPV1 channel. We tested whether varying extracellular K⁺, Ca⁺⁺, and La⁺⁺⁺ concentrations, pH changes, specific agonists or antagonists (with or without added Ca⁺⁺), and the non-specific cation channel blocker Ruthenium Red affected GTTR uptake. All these assays were done for 1 minute or less at room temperature (20° C.), precluding endocytosis. In one set of experiments, we used hydrolyzed Texas Red as a control fluorophore. In another set of experiments, we verified the modulation of GTTR uptake by extracellular La⁺⁺⁺ using immunocytochemistry of unlabeled gentamicin.

Extracellular Potassium

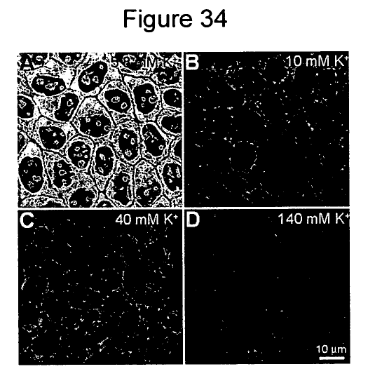

If cationic GTTR penetrates cells via cation channels down an electrochemical gradient, a reduction of the electrical potential difference across the plasma membrane could reduce GTTR uptake. To test this, Hank's balanced salt solution (HBSS) was mixed with varying amounts of equimolar KCl in water. Potassium concentrations ranged from 5.8 mM (HBSS alone) to 140 mM. All solutions were at pH 7.3, and MDCK cells were treated with 1 μg/mL GTTR for 1 minute. Cells treated at 5.8 mM K⁺ show bright cytoplasmic and intra-nuclear GTTR fluorescence (FIG. 34A). Using the same imaging parameters as in FIG. 34A, a clear and considerable decrease in GTTR uptake into cells was observed as the K⁺ concentration increased from 10 mM to 140 mM (FIG. 34B-D). These data suggest that the positive charge of the polyamine gentamicin facilitates the electrophoretic passage of the molecule through cation channels towards the electrically-negative interior of the cells.

Valinomycin

Modulation of Membrane Potential

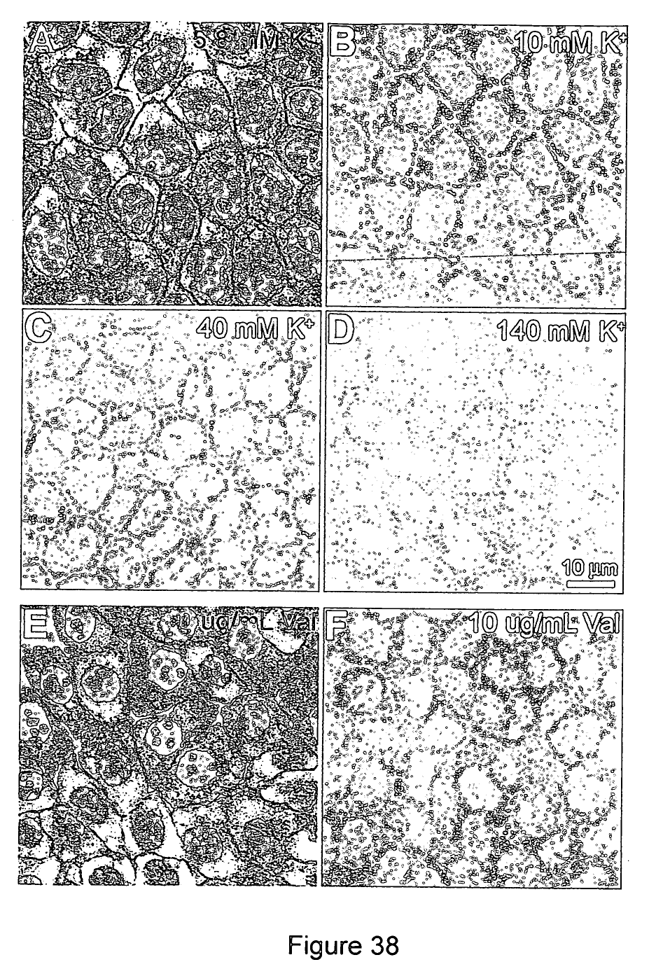

If cationic GTTR penetrates cells via cation channels down an electrochemical gradient, a reduction of the electrical potential difference across the plasma membrane could reduce GTTR uptake. Increases in extracellular potassium serially decrease the negative intracellular resting potential (Zenner, 1986), and reduce the transmembrane cationic driving force into the cell. Hank's balanced salt solution (HBSS) was mixed with varying amounts of equimolar KCl in water. Potassium concentrations ranged from 5.8 mM (HBSS alone) to 140 mM. All solutions were at pH 7.3, and MDCK cells were treated with 1, or 5 μg/mL GTTR for 1 minute, then fixed and rinsed as described. Cells treated at 5.8 mM K⁺ show bright cytoplasmic and intra-nuclear GTTR fluorescence (FIG. 38A). Using the same imaging parameters as in FIG. 38A, a clear and considerable decrease in GTTR uptake into cells was observed as the K⁺ concentration increased from 10 mM to 140 mM (FIG. 38B-D). Valinomycin, a potassium ionophore, also reduces the electrical potential difference across the plasma membrane (Crider et al., 2003; Ren et al., 2001). Cell were rinsed with HBSS and then treated for 30 seconds at pH 7.3 with 5 μg/mL GTTR in HBSS (or without) 10 μg/mL valinomycin. Valinomycin treatment decreased the uptake of GTTR compared to control cells (FIG. 38E,F). These data suggest that the positive charge of the polyamine gentamicin facilitates the electrophoretic passage of the molecule through cation channels towards the electrically-negative interior of the cells.

Regulation of GTTR Uptake:

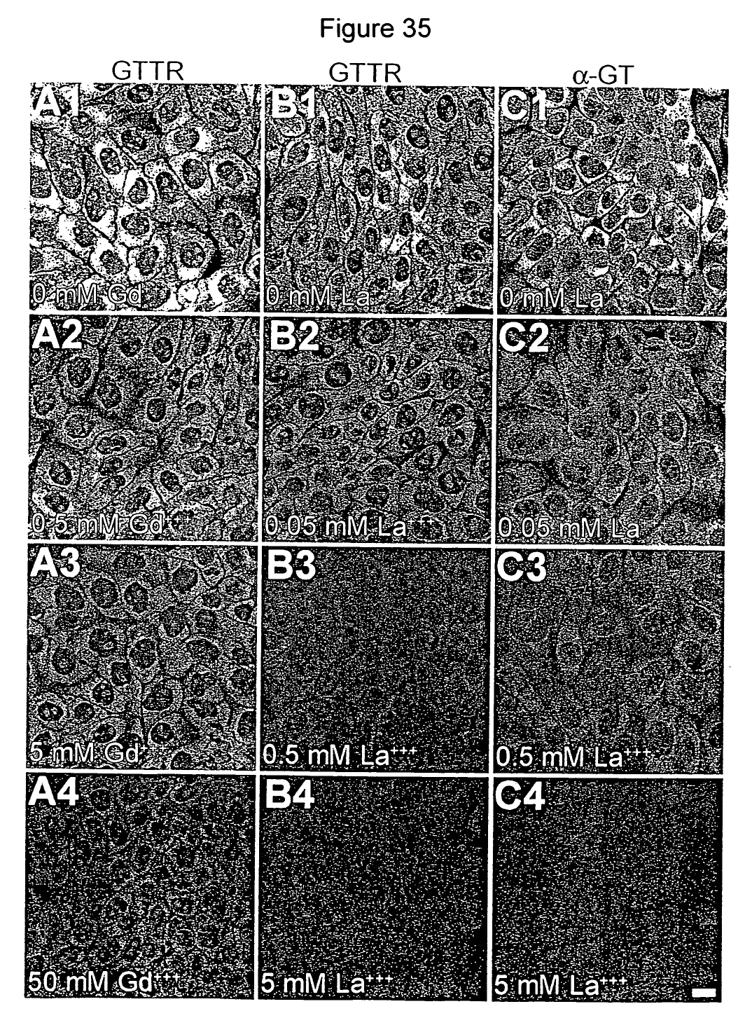

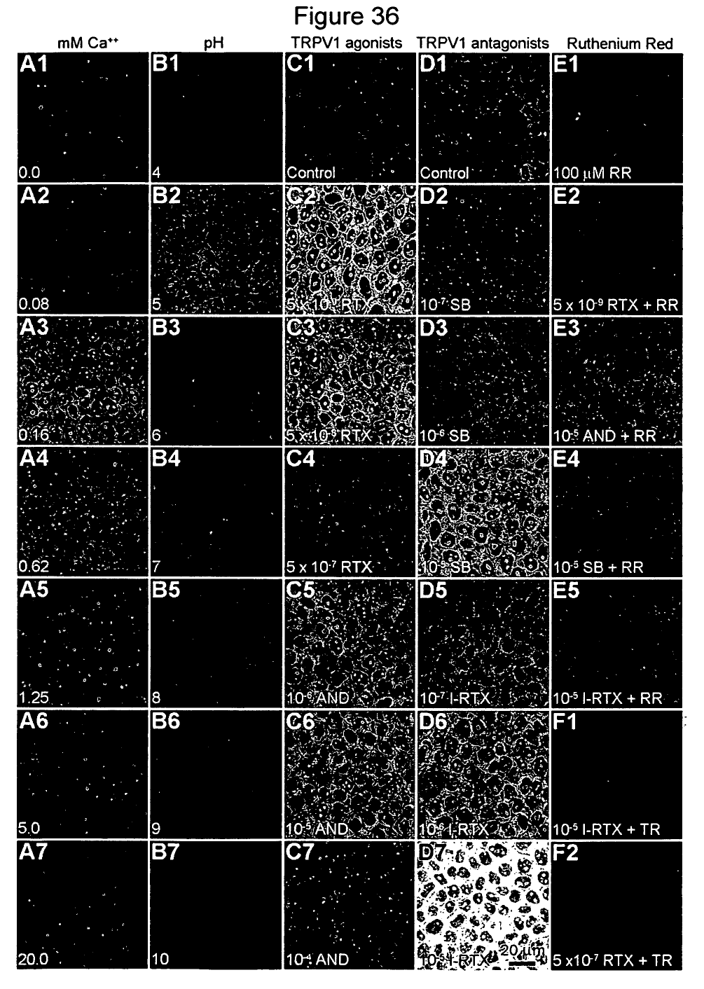

For each of the experimental sets in FIGS. 35 and 36, the same experimental conditions (except as noted), the same lot of GTTR, and the same imaging parameters were used.

Extracellular Trivalent Cations

Gadolinium

Gadolinium blocks calcium-permeant, mechanosensitive cation channels (Kondoh et al., 2003; Urbach et al., 1999). Increasing concentrations of extracellular Gd⁺⁺⁺ decreased GTTR uptake. Cells were washed with HBSS and then treated for 30 seconds at pH 7.3 with 5 mg/mL GTTR in HBSS containing 0, 0.5, 5, or 50 mM Gd⁺⁺⁺. In the absence of Gd⁺⁺⁺, GTTR uptake was high (FIG. 35, A1), but serially decreased at increasing concentrations of Gd⁺⁺⁺ (FIG. 35, C2-C3).

Lanthanum

Lanthanum also blocks non-selective cation channels (Gillo et al., 1996; Walker et al., 2002). Increasing concentrations of extracellular La⁺⁺⁺ decreased GTTR and unconjugated gentamicin uptake. Cells were washed with HBSS and then treated with either 5 mg/mL GTTR or 300 mg/mL unlabeled gentamicin in HBSS for 30 seconds in HBSS containing either 0, 0.5, or 5 mM La⁺⁺⁺, at pH 7.3. When no La⁺⁺⁺ was added, GTTR uptake was high (FIG. 35, B1), but serially decreased with increasing concentrations of La⁺⁺⁺ (FIG. 35, B2-B4). Similarly, in immunocytochemical experiments, bright gentamicin immunolabeling was observed in FATX-fixed cells incubated in the absence of La⁺⁺⁺ (FIG. 35, C1). The intensity of immunofluorescence serially decreased at increasing (0.05, 0.5 and 5 mM) La⁺⁺⁺ concentrations (FIG. 35, C2-C4), verifying modulation of the distribution of GTTR by lanthanum.

The data from both Gd⁺⁺⁺ and La⁺⁺⁺ experimental sets are consistent with the hypothesis that gentamicin uptake can occur through non-selective mono- or divalent cation-permeant channels into the cytoplasm (Hellwig et al., 2004).

Calcium

Changes in extracellular calcium altered GTTR uptake. Cells were washed with 0.9% saline then treated with 5 μg/mL GTTR for 30 seconds in saline at the indicated concentration of calcium chloride, at pH 7.3. When no calcium was added, GTTR uptake was low (FIG. 36, A1), but increased at 0.16 mM calcium (FIG. 36, A3). As calcium concentrations increased above 0.16 mM, GTTR uptake decreased (FIG. 36, A4-A7). These data are consistent with the hypothesis that calcium can compete with gentamicin uptake through calcium-permeant cation channels into the cytoplasm.

Protons

Changes in extracellular pH altered GTTR uptake. Cells were washed with saline and treated with 5 μg/mL GTTR for 30 seconds in buffer at pH ranging from 4 to 10. Although three different buffers, with different calcium concentrations, were used (see methods), in all cases the effect of pH was the same and only the PBS (no calcium added) data are shown. At pH 5 (and to a lesser extent at pH 6) there was increased uptake of GTTR (FIG. 36, B2, B3), consistent with the reported pH range of proton stimulation of inward current through the TRPV1 channel. At pH 4, uptake was lower (FIG. 36, B1). Increasingly basic conditions reduced uptake (FIG. 36, B4-B7). The effects of both calcium and protons on GTTR uptake are consistent with the possibility that TRPV1 channels can play a role in the penetration of gentamicin into the cytoplasm of kidney cells.

TRPV1 Agonists

Resiniferatoxin (RTX) is a potent TRPV1 agonist that induces a transient inward current that is desensitized in the presence of calcium (Acs et al., 1997). We tested the effect of RTX on GTTR penetration of cells to determine whether an agent that opens this channel to a cation current could enhance GTTR uptake. Cells were washed with calcium-free saline and treated with 5 μg/mL GTTR for 30 seconds in the presence of several doses of RTX in calcium-free saline at pH 7.3. At 5×10⁻⁹ M RTX, GTTR uptake was significantly increased (FIG. 36, C2) over the control (FIG. 36, C1). At the higher dose of 5×10⁻⁸ M RTX, uptake was increased, but to a lesser extent (FIG. 36, C3), and at 5×10⁻⁷ M RTX, there was little or no change over control (FIG. 35, C4 and C1, respectively). The decrease in GTTR effect at higher doses might be explained by agonist desensitization due to the residual calcium present (see below).

Anandamide (AND) is an endogenous cannabinoid and TRPV1 agonist that produces a transient inward cation current and competes with both RTX and capsaicin for binding (Olah et al., 2001). It was tested for its effect on GTTR uptake using the same protocol as for RTX. Consistent with its reported weaker binding to TRPV1 (Toth et al., 2003), AND required higher doses to produce increases in GTTR uptake. At 10⁻⁶ M and 10⁻⁵ M AND, GTTR uptake was increased, although not to the level seen with RTX (FIG. 36, C5 and C6, respectively). At 10⁻⁴ M AND, GTTR uptake showed little or no increase over controls (FIG. 36, C7 and C1, respectively). These data show that TRPV1 channel agonists stimulate gentamicin uptake in nominally Ca⁺⁺-free media in a manner similar to their reported stimulation of cation currents (Numazaki et al., 2003).

TRPV1 Antagonists

Two specific TRPV1 antagonists, SB366791 and iodo-RTX, were also tested. Both competitively reduce the binding of known TRPV1 agonists, and block the cation current induced by specific agonists (Gunthorpe et al., 2004; Wahl et al., 2001). Cells were washed with calcium-free saline and treated with 5 μg/mL GTTR for 30 seconds in the presence of SB366791 and iodo-RTX in calcium-free saline at pH 7.3. Surprisingly, both SB366791 and iodo-RTX enhanced GTTR uptake. At doses from 10⁻⁷ M to 10⁻⁵ M, SB366791 serially increased GTTR uptake (FIG. 36, D2-D4). The effect of 1-RTX, which binds to TRPV1 with a higher affinity than SB366791 (Davis et al., 2001; Fowler et al., 2003), was more dramatic (FIG. 36, D5-D7). At 10⁻⁵ M I-RTX the GTTR fluorescence was well over the upper limit of the available 0 to 255 gray scale when using parameters optimized for comparison with the other images in this figure. With both of these molecules, increased doses of these specific antagonists increased uptake (in contrast to the TRPV1 agonists).

To ensure that agonist or antagonist-induced increases in uptake of GTTR was not due to toxicity or increased permeability, we treated cells with hydrolyzed TR at the highest doses shown for both RTX and I-RTX. Neither I-RTX or RTX induced TR penetration into the cytoplasm (FIG. 36, F1, F2).

Non-Specific Cation Channel Blockers

Ruthenium Red (RR) is a non-competitive TRPV1 antagonist that blocks numerous cation channels. Cells treated with 10⁻⁵ M R alone (FIG. 36, E1) took up less GTTR than controls (FIG. 35, D1) The same dose of RR also blocked GTTR increases stimulated by RTX, AND, SB366791, and I-RTX (FIG. 36, E2-E5, respectively), although the AND effect was not completely blocked. Blockade of GTTR uptake by RR further demonstrated the involvement of cation channels in the penetration of GTTR into the cytoplasmic compartment of MDCK cells.

Effect of Calcium on RTX and I-RTX Regulation of GTTR Uptake

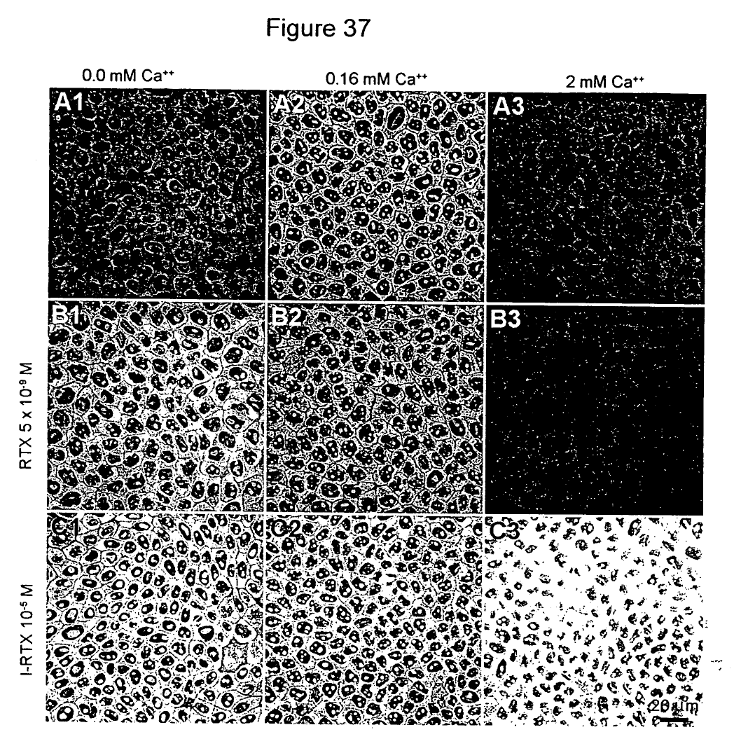

MDCK cells were treated for 30 seconds at room temperature at pH 7.0 with 1 μg/mL of GTTR in 138 mM saline, or saline with 0.16 mM or 2.0 mM calcium at the same osmolarity. In each of these solutions, cells received no other treatment, 5×10⁻⁹ M RTX, or 10⁻⁵ M I-RTX. As in FIG. 36, GTTR uptake was higher at 0.16 mM Ca⁺⁺ (FIG. 37, A2) than at either no calcium added (FIG. 37, A1) or at 2.0 mM (FIG. 37, A3). As also seen in FIG. 36, both 5×10⁻⁹ M RTX (FIG. 37, B1) and 10⁻⁵ M I-RTX (FIG. 37, C1) increased GTTR uptake when in saline (no added calcium buffer). But, in both 0.16 or 2.0 mM added calcium buffers (FIG. 37, B2, B3), RTX reduced GTTR uptake compared to controls at the same calcium levels, while I-RTX still caused increased GTTR uptake in the presence of calcium. These results are consistent with calcium-induced desensitization of the TRPV1 response to its specific agonists, as described previously (Numazaki et al., 2003).

Discussion

Gentamicin (average MW=469) and the conjugate GTTR (MW=approx 1100) are much larger in size than the cations generally envisioned permeating TRP channels. However, a large body of evidence demonstrates that many factors besides size influence permeation of a particular species into a specific channel. These factors include hydration state/hydration energy (Barry et al., 1999; French et al., 1985; Gong et al., 2002; Qu et al., 2000), electrostatic interactions of the permeant with side groups of amino acid residues within the pore (Guidoni et al., 1999), and hydrogen bond exchanges between the permeant and amine side groups which have formed conformational hydrogen bonds with other side groups in the channel pore (Tikhonov et al., 1999). Furthermore, there are numerous reports of large organic cations (including fluorescent dyes) permeating various cation channels, including TRP channels, in inner ear hair cells and transfected kidney cells, with evidence that ionic size is only one of the factors predicting permeability (see discussions in (Corey et al., 2004; Gale et al., 2001; Hellwig et al., 2004; Meyers et al., 2003; Steyger et al., 2003)). Those studies suggest that aminoglycosides, and possibly other polyamine and cationic compounds can permeate cation channels. The data presented in this report provides evidence for fluorescently-labeled gentamicin entering cells via cation channels, and that this penetration can be mediated by regulators of TRPV1, the vanilloid receptor.

Extracellular Potassium

The difference between intracellular potassium concentrations ([K]ᵢ) and extracellular potassium ([K]ₒ) is largely responsible for the electrical potential difference across the plasma membrane. TRP channels, including TRPV1, are not voltage-gated (Benham et al., 2002; Inoue et al., 2003; Vennekens et al., 2002; Voets et al., 2003). Previous studies have shown that aminoglycosides block cation channels of inner ear hair cells with negative resting potentials, but not depolarized hair cells (Kroese et al., 1989). Increasing concentrations of [K]ₒ also depolarizes cells (Gitter, 1993), and reduced GTTR uptake. The toxic effect of high K⁺ is unlikely during a brief exposure at room temperature, and if so would more likely have produced increased (but non-specific), penetration of GTTR down its concentration gradient into the cell. Thus, the simplest explanation for the greater reduction of GTTR uptake at higher [K]ₒ is that the reduced electrical potential difference across the plasma membrane (due to near equi-molar [K]ₒ and [K]_{I}) reduces the electrical driving force for the cationic GTTR to cross the plasma membrane. These data support a model in which gentamicin enters cells electrophoretically via cation channels.

Calcium

The calcium dilution series shown in FIG. 35 (A1-A7) shows that extracellular calcium influences GTTR uptake into cells. A very low level of calcium is necessary for uptake, but even physiological levels (1.8 mM) were inhibitory compared to lower levels, and with higher levels of calcium (>1.8 mM) almost blocking uptake. This could be due to either (i) the two polycations competing for the same channel, (ii) the calcium regulating the open time of the relevant channels, or a combination of both. The enhanced uptake of gentamicin at lower levels of extracellular calcium mimics the greater open probability of TRP channels and inner ear cation transduction channels at lower calcium concentrations (Caterina et al., 1997; Corey et al., 1983; Crawford et al., 1991; Koplas et al., 1997; Ricci et al., 1997). Indeed, the endolymphatic fluids bathing the cation transduction channel on the apical surface of cochlear hair cells (recently shown to contain TRPA1 channel components) typically contain very low calcium concentrations, ˜0.025 mM (Corey et al., 2004; Wangemann et al., 1996). Thus, physiological levels (1.8 mM) of calcium would merely reduce the rate of gentamicin uptake, but not abolish it, as large organic cations could still enter (Hellwig et al., 2004). Thus, the data presented here (at 20° C. to preclude the effects of endocytosis), implicate cation channels as a route for gentamicin to enter the cytoplasmic compartment of cells.

TRPV1 Agonists

Protons, as well as specific agonists that bind to, and compete for, the TRPV1 binding site, produce cation currents though those channels (Vellani et al., 2001). Enhanced uptake of GTTR was observed at pH 5 and reduced at more basic pH levels. The TRPV1 channel cation current is also maximal at pH5 and reduced at more acidic, and particularly more basic pH levels (Vellani et al., 2001). Alternatively, increased protonation at acidic pH could lead to enhanced gentamicin uptake (Lesniak et al., 2003). However, we observed a decreased level of GTTR uptake at pH 4 compared to pH 5, suggesting that increased protonation alone increases uptake. Environmental acidity also increased cisplatin-induced hair cell death (ototoxicity), although whether this was due to the protonation, enhanced uptake or reactivity of the cisplatin molecule to DNA remains to be determined (Tanaka et al., 2003)

In addition to protons, we found that the specific agonists RTX and anandamide both stimulated GTTR uptake in calcium-free media, with a relative effectiveness consistent with their known affinities for the receptor binding site (Olah et al., 2001). For both agonists, higher concentrations of agonist were less effective, suggesting agonist-induced closing or blockage of the channel. This is consistent with the known desensitization of agonist-induced currents (Acs et al., 1997) which occurs when agonists are tested in the presence of calcium. Although our experiments were done in nominally calcium-free buffer, we were unable to use a chelator such as EDTA, because EDTA caused cell islands to detach from the coverglasses and then not be available for observation. Thus, a small amount of calcium was certainly present in our nominally calcium-free buffer.

TRPV1 Antagonists

There are several TRPV1 antagonists which compete with capsaicin or RTX for binding to the TRPV1 receptor. Iodo-RTX binds with high affinity. It induces no current in treated cells, and blocks RTX- or capsaicin-induced currents (Wahl et al., 2001). SB366791 shows similar effects, but with a lower affinity for the binding site than I-RTX (Davis et al., 2001; Fowler et al., 2003). Surprisingly, both of these antagonists significantly increased GTTR uptake, with iodo-RTX more effective at higher doses, consistent with the relative binding affinities of these two molecules. Unlike the agonists RTX and anandamide, no “desensitization” of GTTR uptake was observed at higher concentrations using these antagonists, i.e., higher doses of antagonists induced greater GTTR uptake. Gentamicin is known to bind to PIP₂, a component of the TRPV1 channel, and whose binding to the channel participates in blocking the channel (Chuang et al., 2001; Prescott et al., 2003; Schacht, 1979; Williams et al., 1987). Gentamicin, in its interaction with the channel pore, may bind to and then remove PIP₂ from its pore binding site, opening the channel.

Agonists and Antagonists in the Presence of Ca⁺⁺

FIG. 36 shows that 5×10⁻⁹ M RTX, which stimulates GTTR uptake in Ca⁺⁺-free saline, reduced GTTR uptake in the presence of both low and high doses of Ca⁺⁺ compared to controls at the same Ca⁺⁺ concentrations. Indeed, the combination of 2 mM Ca⁺⁺ and 5×10⁻⁹ RTX greatly reduced GTTR uptake. This effect was not observed with I-RTX. These observations are consistent with the apparent “desensitization” seen in FIG. 36 at higher doses of RTX (but not with I-RTX). This suggests that aminoglycoside penetration of these cells can be both increased or reduced by these regulators of the TRPV1 channel.

Non-Specific Blockade of GTTR Uptake

The non-competitive cation blocker Ruthenium Red reduces GTTR uptake, and blocks the stimulatory effect of both agonists (in calcium-free media) and antagonists, further supporting the conclusion that gentamicin enters cells via one or more cation channels. This also shows that the effect of the specific agonists and antagonists is directly on the cation channels, and not an indirect effect on some other molecular entity.

Significance

All receptors in the growing TRP family are well documented as cation channels. The function we describe here is a departure from the conventional wisdom that these channels are only atomic cation permeant, and also allow the entry of larger molecules like gentamicin, as reported previously for less toxic compounds (Corey et al., 2004; Gale et al., 2001; Hellwig et al., 2004; Meyers et al., 2003). Using fluorescently-labeled gentamicin, and both specific and non-specific modulators of TRPV1, we have provided evidence that that this channel (and/or others with close functional homology) can enable gentamicin entry into the cytoplasm of MDCK cells. In other studies (Steyger et al., 2004) and Example 10, we show that these regulators also modulate gentamicin uptake into inner ear hair cells, the other major cell type susceptible to aminoglycoside toxicity. Blocking aminoglycoside penetration into cells through ion channels offers the possibility of pharmacologically preventing aminoglycoside-induced oto- and nephrotoxicity.

### References for Example 11

Acs, G., Biro, T., Acs, P., Modarres, S., Blumberg, P. M. 1997. Differential activation and desensitization of sensory neurons by resiniferatoxin. J Neurosci 17, 5622-8.
Barry, P. H., Schofield, P. R., Moorhouse, A. J. 1999. Glycine receptors: what gets in and why? Clin Exp Pharmacol Physiol 26, 935-6.
Begg, E. J., Barclay, M. L. 1995. Aminoglycosides—50 years on. Br J Clin Pharmacol 39, 597-603.
Benham, C. D., Davis, J. B., Randall, A. D. 2002. Vanilloid and TRP channels: a family of lipid-gated cation channels. Neuropharmacology 42, 873-88.
Caterina, M. J., Schumacher, M. A., Tominaga, M., Rosen, T. A., Levine, J. D., Julius, D. 1997. The capsaicin receptor: a heat-activated ion channel in the pain pathway. Nature 389, 816-24.
Chuang, H. H., Prescott, E. D., Kong, H., Shields, S., Jordt, S. E., Basbaum, A. I., Chao, M. V., Julius, D. 2001. Bradykinin and nerve growth factor release the capsaicin receptor from PtdIns(4,5)P2-mediated inhibition. Nature 411, 957-62.
Corey, D. P., Hudspeth, A. J. 1983. Kinetics of the receptor current in bullfrog saccular hair cells. J Neurosci 3, 962-76.
Corey, D. P., Garcia-Anoveros, J., Holt, J. R., Kwan, K. Y., Lin, S. Y., Vollrath, M. A., Amalfitano, A., Cheung, E. L., Derfler, B. H., Duggan, A., Geleoc, G. S., Gray, P. A., Hoffman, M. P., Rehm, H. L., Tamasauskas, D., Zhang, D. S. 2004. TRPA1 is a candidate for the mechanosensitive transduction channel of vertebrate hair cells. Nature.
Cortright, D. N., Crandall, M., Sanchez, J. F., Zou, T., Krause, J. E., White, G. 2001. The tissue distribution and functional characterization of human VR1. Biochem Biophys Res Commun 281, 1183-9.
Crawford, A. C., Evans, M. G., Fettiplace, R. 1991. The actions of calcium on the mechano-electrical transducer current of turtle hair cells. J Physiol (Lond) 434, 369-98.
Davis, J. B., Gunthorpe, M. J., Jerman, J. C., Gray, J., Smith, G. D., Davies, C. H., Randall, A. D., Smart, D., Rami, H. K., Wyman, P. A. 2001. Identification of a potent and selective antagonist of vanilloid receptor-1, SB366791. Soc. Neurosci. Abstr. 27, 910.5.
de Jager, P., van Altena, R. 2002. Hearing loss and nephrotoxicity in long-term aminoglycoside treatment in patients with tuberculosis. Int J Tuberc Lung Dis 6, 622-7.
de Lalla, F. 1999. Antimicrobial chemotherapy in the control of surgical infectious complications. J Chemother 11, 440-5.
Fowler, C. J., Jonsson, K. O., Andersson, A., Juntunen, J., Jarvinen, T., Vandevoorde, S., Lambert, D. M., Jerman, J. C., Smart, D. 2003. Inhibition of C6 glioma cell proliferation by anandamide, 1-arachidonoylglycerol, and by a water soluble phosphate ester of anandamide: variability in response and involvement of arachidonic acid. Biochem Pharmacol 66, 757-67.
French, R. J., Shoukimas, J. J. 1985. An ion's view of the potassium channel. The structure of the permeation pathway as sensed by a variety of blocking ions. J Gen Physiol 85, 669-98.
Gale, J. E., Marcotti, W., Kennedy, H. J., Kros, C. J., Richardson, G. P. 2001. FM1-43 dye behaves as a permeant blocker of the hair-cell mechanotransducer channel. J Neurosci 21, 7013-25.
Gitter, A. H. 1993. Cell potential of isolated outer hair cells as a function of extracellular K+ concentration. ORL J Otorhinolaryngol Relat Spec 55, 1-2.
Gong, X., Burbridge, S. M., Cowley, E. A., Linsdell, P. 2002. Molecular determinants of Au(CN)(2)(−) binding and permeability within the cystic fibrosis transmembrane conductance regulator Cl(−) channel pore. J Physiol 540, 39-47.
Goodman, M. B., Schwarz, E. M. 2003. Transducing touch in Caenorhabditis elegans. Annu Rev Physiol 65, 429-52.
Grimm, C., Kraft, R., Sauerbruch, S., Schultz, G., Harteneck, C. 2003. Molecular and functional characterization of the melastatin-related cation channel TRPM3. J Biol Chem 278, 21493-501.
Guidoni, L., Torre, V., Carloni, P. 1999. Potassium and sodium binding to the outer mouth of the K+ channel. Biochemistry 38, 8599-604.
Guler, A. D., Lee, H., Iida, T., Shimizu, I., Tominaga, M., Caterina, M. 2002. Heat-evoked activation of the ion channel, TRPV4. J Neurosci 22, 6408-14.
Gunthorpe, M. J., Rami, H. K., Jerman, J. C., Smart, D., Gill, C. H., Soffin, E. M., Luis Hannan, S., Lappin, S. C., Egerton, J., Smith, G. D., Worby, A., Howett, L., Owen, D., Nasir, S., Davies, C. H., Thompson, M., Wyman, P. A., Randall, A. D., Davis, J. B. 2004. Identification and characterisation of SB-366791, a potent and selective vanilloid receptor (VR1/TRPV1) antagonist. Neuropharmacology 46, 13349.
Hashino, E., Shero, M., Salvi, R. J. 1997. Lysosomal targeting and accumulation of aminoglycoside antibiotics in sensory hair cells. Brain Res 777, 75-85.
Hellwig, N., Plant, T. D., Janson, W., Schafer, M., Schultz, G., Schaefer, M. 2004. TRPV1 acts as proton channel to induce acidification in nociceptive neurons. J Biol Chem 279, 34553-61.
Hofmann, T., Chubanov, V., Gudermann, T., Montell, C. 2003. TRPM5 is a voltage-modulated and Ca(2+)-activated monovalent selective cation channel. Curr Biol 13, 1153-8.
Inoue, R., Hanano, T., Shi, J., Mori, Y., Ito, Y. 2003. Transient receptor potential protein as a novel non-voltage-gated Ca2+ entry channel involved in diverse pathophysiological functions. J Pharmacol Sci 91, 271-6.
Jackson, G. G. 1984. The key role of aminoglycosides in antibacterial therapy and prophylaxis. J Antimicrob Chemother 13 Suppl A, 1-7.
Kahlmeter, G., Dahlager, J. I. 1984. Aminoglycoside toxicity—a review of clinical studies published between 1975 and 1982. J Antimicrob Chemother 13 Suppl A, 9-22.
Keeling, K. M., Bedwell, D. M. 2002. Clinically relevant aminoglycosides can suppress disease-associated premature stop mutations in the IDUA and P53 cDNAs in a mammalian translation system. J Mol Med 80, 367-76.
Koplas, P. A., Rosenberg, R. L., Oxford, G. S. 1997. The role of calcium in the desensitization of capsaicin responses in rat dorsal root ganglion neurons. J Neurosci 17, 3525-37.
Kroese, A. B., Das, A., Hudspeth, A. J. 1989. Blockage of the transduction channels of hair cells in the bullfrog's sacculus by aminoglycoside antibiotics. Hear Res 37, 203-17.
Launay, P., Fleig, A., Perraud, A. L., Scharenberg, A. M., Penner, R., Kinet, J. P. 2002. TRPM4 is a Ca2+-activated nonselective cation channel mediating cell membrane depolarization. Cell 109, 397-407.
Leehey, D. J., Braun, B. I., Tholl, D. A., Chung, L. S., Gross, C. A., Roback, J. A., Lentino, J. R. 1993. Can pharmacokinetic dosing decrease nephrotoxicity associated with aminoglycoside therapy. J Am Soc Nephrol 4, 81-90.
Lesniak, W., Mc Laren, J., Harris, W. R., Pecoraro, V. L., Schacht, J. 2003. An isocratic separation of underivatized gentamicin components, 1H NMR assignment and protonation pattern. Carbohydr Res 338, 2853-62.
Liedtke, W., Choe, Y., Marti-Renom, M. A., Bell, A. M., Denis, C. S., Sai, A., Hudspeth, A. J., Friedman, J. M., Heller, S. 2000. Vanilloid receptor-related osmotically activated channel (VR-OAC), a candidate vertebrate osmoreceptor. Cell 103, 525-35.
Meyers, J. R., MacDonald, R. B., Duggan, A., Lenzi, D., Standaert, D. G., Corwin, J. T., Corey, D. P. 2003. Lighting up the senses: FM1-43 loading of sensory cells through nonselective ion channels. J Neurosci 23, 4054-65.
Mutai, H., Heller, S. 2003. Vertebrate and invertebrate TRPV-like mechanoreceptors. Cell Calcium 33, 471-8.
Nilius, B., Prenen, J., Droogmans, G., Voets, T., Vennekens, R., Freichel, M., Wissenbach, U., Flockerzi, V. 2003. Voltage dependence of the Ca2+-activated cation channel TRPM4. J Biol Chem 278, 30813-20.
Numazaki, M., Tominaga, T., Takeuchi, K., Murayama, N., Toyooka, H., Tominaga, M. 2003. Structural determinant of TRPV 1 desensitization interacts with calmodulin. Proc Natl Acad Sci USA 100, 8002-6.
Olah, Z., Karai, L., Iadarola, M. J. 2001. Anandamide activates vanilloid receptor 1 (VR1) at acidic pH in dorsal root ganglia neurons and cells ectopically expressing VR1. J Biol Chem 276, 31163-70.
Prescott, E. D., Julius, D. 2003. A modular PIP2 binding site as a determinant of capsaicin receptor sensitivity. Science 300, 1284-8.
Qu, Z., Hartzell, H. C. 2000. Anion permeation in Ca(2+)-activated Cl(−) channels. J Gen Physiol 116, 825-44.
Ricci, A. J., Fettiplace, R. 1997. The effects of calcium buffering and cyclic AMP on mechano-electrical transduction in turtle auditory hair cells. J Physiol (Lond) 501, 111-24.
Riccio, A., Mattei, C., Kelsell, R. E., Medhurst, A. D., Calver, A. R., Randall, A. D., Davis, J. B., Benham, C. D., Pangalos, M. N. 2002. Cloning and functional expression of human short TRP7, a candidate protein for store-operated Ca2+ influx. J Biol Chem 277, 12302-9.
Runnels, L. W., Yue, L., Clapham, D. E. 2002. The TRPM7 channel is inactivated by PIP(2) hydrolysis. Nat Cell Biol 4, 329-36.
Rybak, L. P., Kelly, T. 2003. Ototoxicity: bioprotective mechanisms. Curr Opin Otolaryngol Head Neck Surg 11, 328-33.
Sandoval, R. M., Molitoris, B. A. 2004. Gentamicin traffics retrograde through the secretory pathway and is released in the cytosol via the endoplasmic reticulum. Am J Physiol Renal Physiol 286, F617-24.
Schacht, J. 1979. Isolation of an aminoglycoside receptor from guinea pig inner ear tissues and kidney. Arch Otorhinolaryngol 224, 129-34.
Schlingmann, K. P., Weber, S., Peters, M., Niemann Nejsum, L., Vitzthum, H., Klingel, K., Kratz, M., Haddad, E., Ristoff, E., Dinour, D., Syrrou, M., Nielsen, S., Sassen, M., Waldegger, S., Seyberth, H. W., Konrad, M. 2002. Hypomagnesemia with secondary hypocalcemia is caused by mutations in TRPM6, a new member of the TRPM gene family. Nat Genet 31, 166-70.
Schulz, A., Sangkuhl, K., Lennert, T., Wigger, M., Price, D. A., Nuuja, A., Gruters, A., Schultz, G., Schoneberg, T. 2002. Aminoglycoside pretreatment partially restores the function of truncated V(2) vasopressin receptors found in patients with nephrogenic diabetes insipidus. J Clin Endocrinol Metab 87, 5247-57.
Smith, G. D., Gunthorpe, M. J., Kelsell, R. E., Hayes, P. D., Reilly, P., Facer, P., Wright, J. E., Jerman, J. C., Walhin, J. P., Ooi, L., Egerton, J., Charles, K. J., Smart, D., Randall, A. D., Anand, P., Davis, J. B. 2002. TRPV3 is a temperature-sensitive vanilloid receptor-like protein. Nature 418, 186-90.
Steyger, P. S., Peters, S. L., Rehling, J., Hordichok, A., Dai, C. F. 2003. Uptake of gentamicin by bullfrog saccular hair cells in vitro. J Assoc Res Otolaryngol 4, 565-78.
Steyger, P. S., Spencer, N., Hordichok, A. J., Raphael, R. M., Myrdal, S. E. 2004. Reduction of Gentamicin Uptake in Hair Cells in vitro. ARO Midwinter Meeting Abstracts 27, 135.
Story, G. M., Peier, A. M., Reeve, A. J., Eid, S. R., Mosbacher, J., Hricik, T. R., Earley, T. J., Hergarden, A. C., Andersson, D. A., Hwang, S. W., McIntyre, P., Jegla, T., Bevan, S., Patapoutian, A. 2003. ANKTM1, a TRP-like channel expressed in nociceptive neurons, is activated by cold temperatures. Cell 112, 819-29.
Tanaka, F., Whitworth, C. A., Rybak, L. P. 2003. Influence of pH on the ototoxicity of cisplatin: a round window application study. Hear Res 177, 21-31.
Thut, P. D., Wrigley, D., Gold, M. S. 2003. Cold transduction in rat trigeminal ganglia neurons in vitro. Neuroscience 119, 1071-83.
Tikhonov, D. B., Zhorov, B. S., Magazanik, L. G. 1999. Intersegment hydrogen bonds as possible structural determinants of the N/Q/R site in glutamate receptors. Biophys J 77, 1914-26.
Tominaga, M., Caterina, M. J., Malmberg, A. B., Rosen, T. A., Gilbert, H., Skinner, K., Raumann, B. E., Basbaum, A. I., Julius, D. 1998. The cloned capsaicin receptor integrates multiple pain-producing stimuli. Neuron 21, 531-43.
Toth, A., Kedei, N., Wang, Y., Blumberg, P. M. 2003. Arachidonyl dopamine as a ligand for the vanilloid receptor VR1 of the rat. Life Sci 73, 487-98.
Tsiokas, L., Arnould, T., Zhu, C., Kim, E., Walz, G., Sukhatme, V. P. 1999. Specific association of the gene product of PKD2 with the TRPC1 channel. Proc Natl Acad Sci USA 96, 3934-9.
Vellani, V., Mapplebeck, S., Moriondo, A., Davis, J. B., McNaughton, P. A. 2001. Protein kinase C activation potentiates gating of the vanilloid receptor VR1 by capsaicin, protons, heat and anandamide. J Physiol 534, 813-25.
Vennekens, R., Voets, T., Bindels, R. J., Droogmans, G., Nilius, B. 2002. Current understanding of mammalian TRP homologues. Cell Calcium 31, 253-64.
Voets, T., Nilius, B. 2003. TRPs Make Sense. J Membr Biol 192, 1-8.
Wahl, P., Foged, C., Tullin, S., Thomsen, C. 2001. Iodo-resiniferatoxin, a new potent vanilloid receptor antagonist. Mol Pharmacol 59, 9-15.
Wangemann, P., Schacht, J. 1996. Homeostatic mechanisms in the cochlea. In: Dallos, P., Popper, A. N., Fay, R. R., (Eds.), The cochlea. Springer-Verlag, New York. pp. 130-185.
Williams, S. E., Zenner, H. P., Schacht, J. 1987. Three molecular steps of aminoglycoside ototoxicity demonstrated in outer hair cells. Hear Res 30, 11-8.
Wuttke, M. S., Tompkins, L. 2000. Olfactory adaptation in Drosophila larvae. J Neurogenet 14, 43-62.
Xu, H., Zhao, H., Tian, W., Yoshida, K., Roullet, J. B., Cohen, D. M. 2003. Regulation of a transient receptor potential (TRP) channel by tyrosine phosphorylation. SRC family kinase-dependent tyrosine phosphorylation of TRPV4 on TYR-253 mediates its response to hypotonic stress. J Biol Chem 278, 11520-7.
Xu, H., Ramsey, I. S., Kotecha, S. A., Moran, M. M., Chong, J. A., Lawson, D., Ge, P., Lilly, J., Silos-Santiago, I., Xie, Y., DiStefano, P. S., Curtis, R., Clapham, D. E. 2002. TRPV3 is a calcium-permeable temperature-sensitive cation channel. Nature 418, 181-6.
Zheng, J., Dai, C., Steyger, P. S., Kim, Y., Vass, Z., Ren, T., Nuttall, A. L. 2003. Vanilloid receptors in hearing: altered cochlear sensitivity by vanilloids and expression of TRPV1 in the organ of Corti. J Neurophysiol.
Crider, B. P., Xie, X. S. 2003. Characterization of the functional coupling of bovine brain vacuolar-type H(+)-translocating ATPase. Effect of divalent cations, phospholipids, and subunit H(SFD). J Biol Chem 278, 44281-8.
Ren, Y., Yu, H., Wang, H., Carretero, O. A., Garvin, J. L. 2001. Nystatin and valinomycin induce tubuloglomerular feedback. Am J Physiol Renal Physiol 281, F1102-8.
Zenner, H. P. 1986. K+-induced motility and depolarization of cochlear hair cells. Direct evidence for a new pathophysiological mechanism in Meniere's disease. Arch Otorhinolaryngol 243, 108-11
Gillo, B., Chorna, I., Cohen, H., Cook, B., Manistersky, I., Chorev, M., Arnon, A., Pollock, J. A., Selinger, Z., Minke, B. 1996. Coexpression of Drosophila TRP and TRP-like proteins in Xenopus oocytes reconstitutes capacitative Ca2+ entry. Proc Natl Acad Sci USA 93, 14146-51.
Hellwig, N., Plant, T. D., Janson, W., Schafer, M., Schultz, G., Schaefer, M. 2004. TRPV1 acts as proton channel to induce acidification in nociceptive neurons. J Biol Chem 279, 34553-61.
Kondoh, M., Kasai, T., Shimada, M., Kashiwayanagi, M., Yokosawa, H. 2003. cDNA cloning and characterization of an osmotically sensitive TRP channel from ascidian eggs. Comp Biochem Physiol B Biochem Mol Biol 134, 417-23.
Urbach, V., Leguen, I., O'Kelly, I., Harvey, B. J. 1999. Mechanosensitive calcium entry and mobilization in renal A6 cells. J Membr Biol 168, 29-37.
Walker, R. L., Koh, S. D., Sergeant, G. P., Sanders, K. M., Horowitz, B. 2002. TRPC4 currents have properties similar to the pacemaker current in interstitial cells of Cajal. Am J Physiol Cell Physiol 283, C1637-45.

1. A method of preventing injury to the auditory system induced by an ototoxic agent, said method comprising administering to a mammal in need of such treatment a composition that prevents said ototoxic agent's uptake into the cells of the inner ear.

2. The method of claim 1 in which the ototoxic agent is an aminoglycoside antibiotic.

3. The method of claim 1 in which the ototoxic agent is structurally similar to an aminoglycoside antibiotic.

4. The method of claim 1 in which the cells of the inner ear comprise the sensory hair cells of the inner ear.

5. The method of claim 4 in which uptake by hair cells of the inner ear is prevented by removing access of the ototoxic agent to proteins contained in the membranes of the hair cells of the inner ear.

6. The method of claim 5 in which the proteins within the cell membranes comprise ion channel proteins.

7. The method of claim 6 in which the ion channel proteins comprise TRP channels.

8-10. (canceled)

11. A method of preventing injury to the renal system induced by a nephrotoxic agent, said method comprising administering to a mammal in need of such treatment a composition that prevents uptake of said nephrotoxic agent into the cells of the kidney.

12. The method of claim 11 in which the nephrotoxic agent is an antibiotic.

13. The method of claim 12 in which the nephrotoxic agent is an aminoglycoside antibiotic.

14-23. (canceled)

24. A method for assaying the uptake of antibiotics in vitro comprising attaching a fluorescent marker to an antibiotic, applying said fluorescently labeled antibiotic to mammalian cells, using confocal microscopy to follow the uptake of said antibiotic within mammalian cells, and determining cell death subsequent to antibiotic uptake through comparison to cells treated with fluorescent marker alone.

25. The method of claim 24 in which the mammalian cells consist of immortal cell lines.

26. The method of claim 25 in which the immortal cell lines are derived from the kidney.

27. The method of claim 24 in which the mammalian cells comprise primary cell cultures from the inner ear.

28. A method for determining the uptake of pharmaceutical agents by mammalian cells, said method comprising attaching a fluorescent marker to a pharmaceutical agent, attaching said fluorescently labeled pharmaceutical agent to mammalian cells, and then using a fluorescence plate reader to measure uptake of the labeled pharmaceutical agent by the cells.

29. The method in claim 28 in which the pharmaceutical agent is an ototoxic agent.

30. The method in claim 29 in which the ototoxic agent is an antibiotic.

31-41. (canceled)

42. A method for assaying the toxicity of antibiotics, said method comprising the use of a fluorescence plate reader to compare mammalian cells that have received a 6 hour treatment of a pharmaceutical agent or composition of pharmaceutical agents to those that have not.

43. The method of claim 42 in which the pharmaceutical agent comprises an ototoxic agent.

44. The method of claim 43 in which the ototoxic agent is an antibiotic.

45-52. (canceled)
